SEQUENCE LISTING (45 sequences)

SEQ ID NO: 1 - DNA, 20 bp, Artificial
  ′An artificially synthesized primer for PCR
caacagccctgtgaacatgc

SEQ ID NO: 2 - DNA, 21 bp, Artificial
  An artificially synthesized primer for PCR
acccttctggagggaggattc

SEQ ID NO: 3 - DNA, 21 bp, Artificial
  An artificially synthesized primer for PCR
gaggtgatagcattgctttcg

SEQ ID NO: 4 - DNA, 21 bp, Artificial
  An artificially synthesized primer for PCR
caagtcagtgtacaggtaagc

SEQ ID NO: 5 - DNA, 20 bp, Artificial
  An artificially synthesized primer for northern blot probe
cggctaccagaacaagatgg

SEQ ID NO: 6 - DNA, 20 bp, Artificial
  An artificially synthesized primer for northern blot probe
gaagcgcttgtaagggactg

SEQ ID NO: 7 - DNA, 44 bp, Artificial
  An artificially synthesized primer for PCR
ggaattccagaccgtgcatcatgcacatccccaaaagcctccag

SEQ ID NO: 8 - DNA, 34 bp, Artificial
  An artificially synthesized primer for PCR
ccgctcgagcgggttgtcaggcatcatagcaagc

SEQ ID NO: 9 - DNA, 19 bp, Artificial
  An artificially synthesized target sequence for siRNA
gaagcagcacgacttcttc

SEQ ID NO: 10 - DNA, 19 bp, Artificial
  An artificially synthesized target sequence for siRNA
cgtacgcggaatacttcga

SEQ ID NO: 11 - DNA, 19 bp, Artificial
  An artificially synthesized target sequence for siRNA
caagatggagtctccgcag

SEQ ID NO: 12 - DNA, 19 bp, Artificial
  An artificially synthesized target sequence for siRNA
atgatgctccagtccctta

SEQ ID NO: 13 - DNA, 1009 bp, Homo sapiens
ggcgaaaggaagggcgggccctcaggtgacgcagattggccctccagagaaggggcggccccttctcaggtgccgtcacccccaaacctcagcactgccattccaggtccttaaaggggagaccgcgattctaagaagcagccaagcccagggctggcctgaagcccccggacggcagtgcccagcaggcagcgcccagcaccctggctcccacctcccagtggccccaaaggaaaacagctgccatgcacatccccaaaagcctccaggagctggccaacagcgaagccgtgcagtttctgagaaggcccaagaccatcacgcgggtcttcgaaggggtcttctccctgatcgtcttctcctccctgctgaccgacggctaccagaacaagatggagtctccgcagctccactgcattctcaacagcaacagcgtggcctgcagctttgccgtgggagccggcttcctggccttcctcagctgcctggccttcctcgtcctggacacacaggagacccgcattgccggcacccgcttcaagacagccttccagctcctggacttcatcctggctgttctctgggcagttgtctggttcatgggtttctgcttcctggccaaccaatggcagcattcgccgcccaaagagttcctcctggggagcagcagtgcccaggcagccatcgccttcaccttcttctccatccttgtctggatattccaggcctacctggcattccaggacctccgaaatgatgctccagtcccttacaagcgcttcctggatgagggtggcatggtgctgaccaccctccccttgccctctgccaacagccctgtgaacatgcccaccactggccccaacagcctgagttatgctagctctgccctgtccccctgtctgaccgctccaaagtccccccggcttgctatgatgcctgacaactaaatatccttatccaaatcaataaagagagaatcctccctccagaagggtttctaaaaaca

SEQ ID NO: 14 - protein, 234 aa, Homo sapiens
MHIPKSLQELANSEAVQFLRRPKTITRVFEGVFSLIVFSSLLTDGYQNKMESPQLHCILNSNSVACSFAVGAGFLAFLSCLAFLVLDTQETRIAGTRFKTAFQLLDFILAVLWAVVWFMGFCFLANQWQHSPPKEFLLGSSSAQAAIAFTFFSILVWIFQAYLAFQDLRNDAPVPYKRFLDEGGMVLTTLPLPSANSPVNMPTTGPNSLSYASSALSPCLTAPKSPRLAMMPDN

SEQ ID NO: 15 - protein, 16 aa, Artificial
  ′An artificially synthesized epitope peptide sequence
LDEESILKQEHHHHHH

SEQ ID NO: 16 - protein, 22 aa, Artificial
  An artificially synthesized epitope peptide sequence
DYKDHDGDYKDHDIDYKDDDDK

SEQ ID NO: 17 - DNA, 19 bp, Artificial
  An artificially synthesized primer for PCR
cgacggcttccagaacaag

SEQ ID NO: 18 - DNA, 19 bp, Artificial
  An artificially synthesized primer for PCR
cttgttctggaagccgtcg

SEQ ID NO: 19 - DNA, 19 bp, Artificial
  An artificially synthesized target sequence for siRNA
cgcauugccggcacccgcu

SEQ ID NO: 20 - DNA, 19 bp, Artificial
  An artificially synthesized target sequence for siRNA
gcauugccggcacccgcuu

SEQ ID NO: 21 - DNA, 19 bp, Artificial
  An artificially synthesized target sequence for siRNA
caaacauugugaauuacuu

SEQ ID NO: 22 - DNA, 3303 bp, Homo sapiens
agttctcgcgggacaccgacggggagcggaagccaggaggtattgctgcttcggcgaccgggcggcggcagcggcggcggcggctgtggcagagtctgtgcctgtggcggtgacggcggcgggagcaagcgctgccctcgcagagcagccttggggtcgccggccgctcgcagcgttgtggaggggcgggccggacgctgagcggagcagctgcgccacgggtggcattgtgtgtcccagagtgccggagcgagtcccagaagagaggcgaggctaagcccagagcgctgggttgcttcagcagggaagactcccttccccctgcttcaggctgctgagcactgagcagcgctcagaatggaagccatcgccaaatatgacttcaaagctactgcagacgacgagctgagcttcaaaaggggggacatcctcaaggttttgaacgaagaatgtgatcagaactggtacaaggcagagcttaatggaaaagacggcttcattcccaagaactacatagaaatgaaaccacatccgtggttttttggcaaaatccccagagccaaggcagaagaaatgcttagcaaacagcggcacgatggggcctttcttatccgagagagtgagagcgctcctggggacttctccctctctgtcaagtttggaaacgatgtgcagcacttcaaggtgctccgagatggagccgggaagtacttcctctgggtggtgaagttcaattctttgaatgagctggtggattatcacagatctacatctgtctccagaaaccagcagatattcctgcgggacatagaacaggtgccacagcagccgacatacgtccaggccctctttgactttgatccccaggaggatggagagctgggcttccgccggggagattttatccatgtcatggataactcagaccccaactggtggaaaggagcttgccacgggcagaccggcatgtttccccgcaattatgtcacccccgtgaaccggaacgtctaagagtcaagaagcaattatttaaagaaagtgaaaaatgtaaaacacatacaaaagaattaaacccacaagctgcctctgacagcagcctgtgagggagtgcagaacacctggccgggtcaccctgtgaccctctcactttggttggaactttagggggtgggagggggcgttggatttaaaaatgccaaaacttacctataaattaagaagagtttttattacaaattttcactgctgctcctctttcccctcctttgtcttttttttcatccttttttctcttctgtccatcagtgcatgacgtttaaggccacgtatagtcctagctgacgccaataataaaaaacaagaaaccaagtgggctggtattctctctatgcaaaatgtctgttttagttggaatgactgaaagaagaacagctgttcctgtgttcttcgtatatacacacaaaaaggagcgggcagggccgctcgatgcctttgctgtttagcttcctccagaggaggggacttgtaggaatctgccttccagcccagacccccagtgtattttgtccaagttcacagtagagtagggtagaaggaaagcatgtctctgcttccatggcttcctgagaaagcccacctgggctgggcgcggtggctcacgcctgtaatcccagcactttgggaggccaaggtgggcggatcacaaggtcaggagttcgagaccaacctagccaacatggtgaaaccccgtctctactaaaaataagaaattagccgggtgtggcacgcacctgtagtcccagctacttgggagcctgaggcaggagaatcgcttgaacctgggaagtggaggttgagtgagccgggaccgtgccattgtactccagcctgggtgacagagcgagattccgtctcaaaaaaaaaaaaaaaaagcccacctgaaagcctgtctctttccactttgttggcccttccagtgggattatcgagcatgttgttttttcatagtgcctttttccttatttcaagggttgcttctgagtggtgttttttttttttttttaatttgttttgttttaaaataagttaaaggcagtccagagcttttcagccaatttgtctcctactctgtgtaaatatttttccctccgggcaggggagccagggtagagcaaaggagacaaagcaggagtggaaggtgaggcgttctcctgcttgtactaagccaggaggctttaagctccagctttaagggttgtgagccccttgggggttcagggaactgcttgcccagggtgcagtgtgagtgtgatgggccaccggggcaagagggaaggtgaccgcccagctctcccacatcccactggatctggcttacaggggggtcggaagcctgtcctcaccgtctcgggggttgtggcccccgccccctccctatatgcacccctggaaccagcaagtcccagacaaggagagcggaggaggaagtcatgggaacgcagcctccagttgtagcaggtttcactattcctatgctggggtacacagtgagagtactcacttttcacttgtcttgctcttagattgggccatggctttcatcctgtgtcccctgacctgtccaggtgagtgtgagggcagcactgggaagctggagtgctgcttgtgcctcccttcccagtgggctgtgttgactgctgctccccacccctaccgatggtcccaggaagcagggagagttggggaaggcaagattggaaagacaggaagaccaaggcctcggcagaactctctgtcttctctccacttctggtcccctgtggtgatgtgcctgtaatctttttctccacccaaaccccttcccacgacaaaaacaagactgcctccctctcttccgggagctggtgacagccttgggcctttcagtcccaaagcggccgatgggagtctccctccgactccagatatgaacagggcccaggcctggagcgtttgctgtgccaggaggcggcagctcttctgggcagagcctgtccccgccttccctcactcttcctcatcctgcttctcttttcctcgcagatgataaaaggaatctggcattctacacctggaccatttgattgttttattttggaattggtgtatatcatgaagccttgctgaactaagttttgtgtgtatatatttaaaaaaaaaatcagtgtttaaataaagacctatgtacttaatcctttaactctgcggatagcatttggtaggtagtgattaactgtgaataataaatacacaatgaattcttca

SEQ ID NO: 23 - protein, 217 aa, Homo sapiens [sequence omitted]

SEQ ID NO: 24 - DNA, 3181 bp, Homo sapiens
aagttctcgcgggacaccgacggggagcggaagccaggaggtattgctgcttcggcgaccgggcggcggcagcggcggcggcggctgtggcagagtctgtgcctgtggcggtgacggcggcgggagcaagcgctgccctcgcagagcagccttggggtcgccggccgctcgcagcgttgtggaggggcgggccggacgctgagcggagcagctgcgccacgggtggcattgtgtgtcccagagtgccggagcgagtcccagaagagaggcgaggctaagcccagagcgctgggttgcttcagcagggaagactcccttccccctgcttcaggctgctgagcactgagcagcgctcagaatggaagccatcgccaaatatgacttcaaagctactgcagacgacgagctgagcttcaaaaggggggacatcctcaaggttttgaacgaagaatgtgatcagaactggtacaaggcagagcttaatggaaaagacggcttcattcccaagaactacatagaaatgaaaccacatccgtttggaaacgatgtgcagcacttcaaggtgctccgagatggagccgggaagtacttcctctgggtggtgaagttcaattctttgaatgagctggtggattatcacagatctacatctgtctccagaaaccagcagatattcctgcgggacatagaacaggtgccacagcagccgacatacgtccaggccctctttgactttgatccccaggaggatggagagctgggcttccgccggggagattttatccatgtcatggataactcagaccccaactggtggaaaggagcttgccacgggcagaccggcatgtttccccgcaattatgtcacccccgtgaaccggaacgtctaagagtcaagaagcaattatttaaagaaagtgaaaaatgtaaaacacatacaaaagaattaaacccacaagctgcctctgacagcagcctgtgagggagtgcagaacacctggccgggtcaccctgtgaccctctcactttggttggaactttagggggtgggagggggcgttggatttaaaaatgccaaaacttacctataaattaagaagagtttttattacaaattttcactgctgctcctctttcccctcctttgtcttttttttcatccttttttctcttctgtccatcagtgcatgacgtttaaggccacgtatagtcctagctgacgccaataataaaaaacaagaaaccaagtgggctggtattctctctatgcaaaatgtctgttttagttggaatgactgaaagaagaacagctgttcctgtgttcttcgtatatacacacaaaaaggagcgggcagggccgctcgatgcctttgctgtttagcttcctccagaggaggggacttgtaggaatctgccttccagcccagacccccagtgtattttgtccaagttcacagtagagtagggtagaaggaaagcatgtctctgcttccatggcttcctgagaaagcccacctgggctgggcgcggtggctcacgcctgtaatcccagcactttgggaggccaaggtgggcggatcacaaggtcaggagttcgagaccaacctagccaacatggtgaaaccccgtctctactaaaaataagaaattagccgggtgtggcacgcacctgtagtcccagctacttgggagcctgaggcaggagaatcgcttgaacctgggaagtggaggttgagtgagccgggaccgtgccattgtactccagcctgggtgacagagcgagattccgtctcaaaaaaaaaaaaaaaaagcccacctgaaagcctgtctctttccactttgttggcccttccagtgggattatcgagcatgttgttttttcatagtgcctttttccttatttcaagggttgcttctgagtggtgttttttttttttttttaatttgttttgttttaaaataagttaaaggcagtccagagcttttcagccaatttgtctcctactctgtgtaaatatttttccctccgggcaggggagccagggtagagcaaaggagacaaagcaggagtggaaggtgaggcgttctcctgcttgtactaagccaggaggctttaagctccagctttaagggttgtgagccccttgggggttcagggaactgcttgcccagggtgcagtgtgagtgtgatgggccaccggggcaagagggaaggtgaccgcccagctctcccacatcccactggatctggcttacaggggggtcggaagcctgtcctcaccgtctcgggggttgtggcccccgccccctccctatatgcacccctggaaccagcaagtcccagacaaggagagcggaggaggaagtcatgggaacgcagcctccagttgtagcaggtttcactattcctatgctggggtacacagtgagagtactcacttttcacttgtcttgctcttagattgggccatggctttcatcctgtgtcccctgacctgtccaggtgagtgtgagggcagcactgggaagctggagtgctgcttgtgcctcccttcccagtgggctgtgttgactgctgctccccacccctaccgatggtcccaggaagcagggagagttggggaaggcaagattggaaagacaggaagaccaaggcctcggcagaactctctgtcttctctccacttctggtcccctgtggtgatgtgcctgtaatctttttctccacccaaaccccttcccacgacaaaaacaagactgcctccctctcttccgggagctggtgacagccttgggcctttcagtcccaaagcggccgatgggagtctccctccgactccagatatgaacagggcccaggcctggagcgtttgctgtgccaggaggcggcagctcttctgggcagagcctgtccccgccttccctcactcttcctcatcctgcttctcttttcctcgcagatgataaaaggaatctggcattctacacctggaccatttgattgttttattttggaattggtgtatatcatgaagccttgctgaactaagttttgtgtgtatatatttaaaaaaaaaatcagtgtttaaataaagacctatgtacttaatcctttaactctgcggatagcatttggtaggtagtgattaactgtgaataataaatacacaatgaattcttca

SEQ ID NO: 25 - protein, 176 aa, Homo sapiens
MEAIAKYDFKATADDELSFKRGDILKVLNEECDQNWYKAELNGKDGFIPKNYIEMKPHPFGNDVQHFKVLRDGAGKYFLWVVKFNSLNELVDYHRSTSVSRNQQIFLRDIEQVPQQPTYVQALFDFDPQEDGELGFRRGDFIHVMDNSDPNWWKGACHGQTGMFPRNYVTPVNRNV

SEQ ID NO: 26 - DNA, 3484 bp, Homo sapiens
tgccctcccgcggctgcagccggagccgaaggtggtggctgcacagtagacgccccctcacggcttcccccacacgctcccgccccctcgctcgcccatcgcgcttccctcacaggctctgcagtcctcccccacagacgccttcccccttggactctcattcccttttccacggagccccgcgctttcgtgagccccctcgaggaacctggtctccgcatccagttaccacctcctgcctcagaggccatctgagcccttcgcacctcgcccctcagtccccccttccccccccgcccgcgtcgcctcgctccctcccgcccccccatcatcccttccctcgcagttcccctgtcctgaggggagccccgccacgggcagcgcggcggcggcggcaggagggagaaagtgaagcggtagctcgcgcacactcgcgccctcactcccggctaggcggcacccaccgccgggaggaggaggaggagccgagaggagctgagcgagcgcggaagtagctgctgctggtggtgacaatgtcaaataacggcctagacattcaagacaaacccccagcccctccgatgagaaataccagcactatgattggagccggcagcaaagatgctggaaccctaaaccatggttctaaacctctgcctccaaacccagaggagaagaaaaagaaggaccgattttaccgatccattttacctggagataaaacaaataaaaagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagctgaggattacaattcttctaatgccttgaatgtgaaggctgtgtctgagactcctgcagtgccaccagtttcagaagatgaggatgatgatgatgatgatgctaccccaccaccagtgattgctccacgcccagagcacacaaaatctgtatacacacggtctgtgattgaaccacttcctgtcactccaactcgggacgtggctacatctcccatttcacctactgaaaataacaccactccaccagatgctttgacccggaatactgagaagcagaagaagaagcctaaaatgtctgatgaggagatcttggagaaattacgaagcatagtgagtgtgggcgatcctaagaagaaatatacacggtttgagaagattggacaaggtgcttcaggcaccgtgtacacagcaatggatgtggccacaggacaggaggtggccattaagcagatgaatcttcagcagcagcccaagaaagagctgattattaatgagatcctggtcatgagggaaaacaagaacccaaacattgtgaattacttggacagttacctcgtgggagatgagctgtgggttgttatggaatacttggctggaggctccttgacagatgtggtgacagaaacttgcatggatgaaggccaaattgcagctgtgtgccgtgagtgtctgcaggctctggagttcttgcattcgaaccaggtcattcacagagacatcaagagtgacaatattctgttgggaatggatggctctgtcaagctaactgactttggattctgtgcacagataaccccagagcagagcaaacggagcaccatggtaggaaccccatactggatggcaccagaggttgtgacacgaaaggcctatgggcccaaggttgacatctggtccctgggcatcatggccatcgaaatgattgaaggggagcctccatacctcaatgaaaaccctctgagagccttgtacctcattgccaccaatgggaccccagaacttcagaacccagagaagctgtcagctatcttccgggactttctgaaccgctgtctcgagatggatgtggagaagagaggttcagctaaagagctgctacaggtgagaaaactgaggtttcaagtgtttagtaacttttccatgatagctgcatcaattcctgaagattgccaagcccctctccagcctcactccactgattgctgcagctaaggaggcaacaaagaacaatcactaaaaccacactcaccccagcctcattgtgccaagccttctgtgagataaatgcacatttcagaaattccaactcctgatgccctcttctccttgccttgcttctcccatttcctgatctagcactcctcaagactttgatccttggaaaccgtgtgtccagcattgaagagaactgcaactgaatgactaatcagatgatggccatttctaaataaggaatttcctcccaattcatggatatgagggtggtttatgattaagggtttatataaataaatgtttctagtcttccgtgtgtcaaaatcctcacctccttcataaccatctcccacaattaattcttgactatataaatttatggtttgataatattatcaatttgtaatcaattgagatttctttagtgcttgcttttctgtgactcaactgcccagacacctcattgtacttgaaaactggaacagcttgggaatgccatggggtttgataatctgccagggacatgaagaggctcagcttcctggaccatgactttggctcagctgatcctgacatgggagaacaaccacatttttctttgtgtgtgcttctagcagctgttcgggaggaccttgacccaatagtgttcccatgctgtttcttgtgaaatgctctcggctatgtagcagcttttgattccctgcataccctaggctgctgcccctatcctgtcccttgtttataacattgagaggttttctagggcacatactgagtgagagcagtgttgagaagtcggggaaaatggtgactacttttagagcaaggctgggcatcagcacctgtccagctctacttgtgtgatgtttcaggaactcagcccctttttctgcctaggataaggagctgaaagattaacttggatcttctaatggtccaaatcttttggtcacaataaagagtctccaaattagagactgcatgttagttctggatggatttggtggcctgacatgataccctgccagctgtgaggggaccccgtttttaagatgcatggccaagctctctgcaaatggaaatgcttacactgggtgttggggatgtttgctacctcctgctatttttgtggttttggttctcccactatggtaggacccctggccagcattgtggcttgtcatgtcagccccattgactaccttctcatgctctgaggtactactgcctctgcagcacaaatttctatttctgtcaataaaaggagatgaaaatattctaaaaaa

SEQ ID NO: 27 - protein, 553 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQVRKLRFQVFSNFSMIAASIPEDCQAPLQPHSTDCCS

SEQ ID NO: 28 - DNA, 3435 bp, Homo sapiens
tgccctcccgcggctgcagccggagccgaaggtggtggctgcacagtagacgccccctcacggcttcccccacacgctcccgccccctcgctcgcccatcgcgcttccctcacaggctctgcagtcctcccccacagacgccttcccccttggactctcattcccttttccacggagccccgcgctttcgtgagccccctcgaggaacctggtctccgcatccagttaccacctcctgcctcagaggccatctgagcccttcgcacctcgcccctcagtccccccttccccccccgcccgcgtcgcctcgctccctcccgcccccccatcatcccttccctcgcagttcccctgtcctgaggggagccccgccacgggcagcgcggcggcggcggcaggagggagaaagtgaagcggtagctcgcgcacactcgcgccctcactcccggctaggcggcacccaccgccgggaggaggaggaggagccgagaggagctgagcgagcgcggaagtagctgctgctggtggtgacaatgtcaaataacggcctagacattcaagacaaacccccagcccctccgatgagaaataccagcactatgattggagccggcagcaaagatgctggaaccctaaaccatggttctaaacctctgcctccaaacccagaggagaagaaaaagaaggaccgattttaccgatccattttacctggagataaaacaaataaaaagaaagagaaagagcggccagagatttctctcccttcagattttgaacacacaattcatgtcggttttgatgctgtcacaggggagtttacgggaatgccagagcagtgggcccgcttgcttcagacatcaaatatcactaagtcggagcagaagaaaaacccgcaggctgttctggatgtgttggagttttacaactcgaagaagacatccaacagccagaaatacatgagctttacagataagtcagctgaggattacaattcttctaatgccttgaatgtgaaggctgtgtctgagactcctgcagtgccaccagtttcagaagatgaggatgatgatgatgatgatgctaccccaccaccagtgattgctccacgcccagagcacacaaaatctgtatacacacggtctgtgattgaaccacttcctgtcactccaactcgggacgtggctacatctcccatttcacctactgaaaataacaccactccaccagatgctttgacccggaatactgagaagcagaagaagaagcctaaaatgtctgatgaggagatcttggagaaattacgaagcatagtgagtgtgggcgatcctaagaagaaatatacacggtttgagaagattggacaaggtgcttcaggcaccgtgtacacagcaatggatgtggccacaggacaggaggtggccattaagcagatgaatcttcagcagcagcccaagaaagagctgattattaatgagatcctggtcatgagggaaaacaagaacccaaacattgtgaattacttggacagttacctcgtgggagatgagctgtgggttgttatggaatacttggctggaggctccttgacagatgtggtgacagaaacttgcatggatgaaggccaaattgcagctgtgtgccgtgagtgtctgcaggctctggagttcttgcattcgaaccaggtcattcacagagacatcaagagtgacaatattctgttgggaatggatggctctgtcaagctaactgactttggattctgtgcacagataaccccagagcagagcaaacggagcaccatggtaggaaccccatactggatggcaccagaggttgtgacacgaaaggcctatgggcccaaggttgacatctggtccctgggcatcatggccatcgaaatgattgaaggggagcctccatacctcaatgaaaaccctctgagagccttgtacctcattgccaccaatgggaccccagaacttcagaacccagagaagctgtcagctatcttccgggactttctgaaccgctgtctcgagatggatgtggagaagagaggttcagctaaagagctgctacagcatcaattcctgaagattgccaagcccctctccagcctcactccactgattgctgcagctaaggaggcaacaaagaacaatcactaaaaccacactcaccccagcctcattgtgccaagccttctgtgagataaatgcacatttcagaaattccaactcctgatgccctcttctccttgccttgcttctcccatttcctgatctagcactcctcaagactttgatccttggaaaccgtgtgtccagcattgaagagaactgcaactgaatgactaatcagatgatggccatttctaaataaggaatttcctcccaattcatggatatgagggtggtttatgattaagggtttatataaataaatgtttctagtcttccgtgtgtcaaaatcctcacctccttcataaccatctcccacaattaattcttgactatataaatttatggtttgataatattatcaatttgtaatcaattgagatttctttagtgcttgcttttctgtgactcaactgcccagacacctcattgtacttgaaaactggaacagcttgggaatgccatggggtttgataatctgccagggacatgaagaggctcagcttcctggaccatgactttggctcagctgatcctgacatgggagaacaaccacatttttctttgtgtgtgcttctagcagctgttcgggaggaccttgacccaatagtgttcccatgctgtttcttgtgaaatgctctcggctatgtagcagcttttgattccctgcataccctaggctgctgcccctatcctgtcccttgtttataacattgagaggttttctagggcacatactgagtgagagcagtgttgagaagtcggggaaaatggtgactacttttagagcaaggctgggcatcagcacctgtccagctctacttgtgtgatgtttcaggaactcagcccctttttctgcctaggataaggagctgaaagattaacttggatcttctaatggtccaaatcttttggtcacaataaagagtctccaaattagagactgcatgttagttctggatggatttggtggcctgacatgataccctgccagctgtgaggggaccccgtttttaagatgcatggccaagctctctgcaaatggaaatgcttacactgggtgttggggatgtttgctacctcctgctatttttgtggttttggttctcccactatggtaggacccctggccagcattgtggcttgtcatgtcagccccattgactaccttctcatgctctgaggtactactgcctctgcagcacaaatttctatttctgtcaataaaaggagatgaaaatattctaaaaaa

SEQ ID NO: 29 - protein, 545 aa, Homo sapiens
MSNNGLDIQDKPPAPPMRNTSTMIGAGSKDAGTLNHGSKPLPPNPEEKKKKDRFYRSILPGDKTNKKKEKERPEISLPSDFEHTIHVGFDAVTGEFTGMPEQWARLLQTSNITKSEQKKNPQAVLDVLEFYNSKKTSNSQKYMSFTDKSAEDYNSSNALNVKAVSETPAVPPVSEDEDDDDDDATPPPVIAPRPEHTKSVYTRSVIEPLPVTPTRDVATSPISPTENNTTPPDALTRNTEKQKKKPKMSDEEILEKLRSIVSVGDPKKKYTRFEKIGQGASGTVYTAMDVATGQEVAIKQMNLQQQPKKELIINEILVMRENKNPNIVNYLDSYLVGDELWVVMEYLAGGSLTDVVTETCMDEGQIAAVCRECLQALEFLHSNQVIHRDIKSDNILLGMDGSVKLTDFGFCAQITPEQSKRSTMVGTPYWMAPEVVTRKAYGPKVDIWSLGIMAIEMIEGEPPYLNENPLRALYLIATNGTPELQNPEKLSAIFRDFLNRCLEMDVEKRGSAKELLQHQFLKIAKPLSSLTPLIAAAKEATKNNH

SEQ ID NO: 30 - DNA, 3291 bp, Homo sapiens
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 31 - protein, 648 aa, Homo sapiens [sequence omitted]

SEQ ID NO: 32 - DNA, 2603 bp, Homo sapiens
aggcgaggcttccccttccccgcccctcccccggcctccagtccctcccagggccgcttcgcagagcggctaggagcacggcggcggcggcactttccccggcaggagctggagctgggctctggtgcgcgcgcggctgtgccgcccgagccggagggactggttggttgagagagagagaggaagggaatcccgggctgccgaaccgcacgttcagcccgctccgctcctgcagggcagcctttcggctctctgcgcgcgaagccgagtcccgggcgggtggggcgggggtccactgagaccgctaccggcccctcggcgctgacgggaccgcgcggggcgcacccgctgaaggcagccccggggcccgcggcccggacttggtcctgcgcagcgggcgcggggcagcgcagcgggaggaagcgagaggtgctgccctccccccggagttggaagcgcgttacccgggtccaaaatgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaagtgtttgggaagcaacaaagagcgagtcccctgcccggtggtttgccatgtcgcttttgggcctccttcccatgcctgtctctgttcagatgtgcatttcacctgtgacaaaggatgaagaacacagcatgtgccaagattctactcttgtcatttttaatattactgtctttattcttattactattattgttcccctaagtggattggctttgtgcttggggctatttgtgtgtatgctgatgatcaaaacctgtgccaggctgaattacagtgaaattttggtgaatgtgggtagtcattcttacaattgcactgctgttcctgctccatgactggctgtctgcctgtattttcgggattctttgacatttggtggtactttattcttgctgggcatactttctctctaggagggagccttgtgagatccttcacaggcagtgcatgtgaagcatgctttgctgctatgaaaatgagcatcagagagtgtacatcatgttattttattattattatttgcttttcatgtagaactcagcagttgacatccaaatctagccagagcccttcactgccatgatagctggggcttcaccagtctgtctactgtggtgatctgtagacttctggttgtatttctatatttattttcagtatactgtgtgggatacttagtggtatgtctctttaagttttgattaatgtttcttaaatggaattattttgaatgtcacaaattgatcaagatattaaaatgtcggatttatctttccccatatccaagtaccaatgctgttgtaaacaacgtgtatagtgcctaaaattgtatgaaaatccttttaaccattttaacctagatgtttaacaaatctaatctcttattctaataaatatactatgaaataaaaaaaaaaggatgaaagctaaaaaaaaaaaaaaaaaa

SEQ ID NO: 33 - protein, 393 aa, Homo sapiens [sequence omitted]

SEQ ID NO: 34 - DNA, 1759 bp, Homo sapiens
cccctgcctctcggactcgggctgcggcgtcagccttcttcgggcctcggcagcggtagcggctcgctcgcctcagccccagcgcccctcggctaccctcggcccaggcccgcagcgccgcccgccctcggccgccccgacgccggcctgggccgcggccgcagccccgggctcgcgtaggcgccgaccgctcccggcccgccccctatgggccccggctagaggcgccgccgccgccggcccgcggagccccgatgctggcccggaggaagccggtgctgccggcgctcaccatcaaccctaccatcgccgagggcccatcccctaccagcgagggcgcctccgaggcaaacctggtggacctgcagaagaagctggaggagctggaacttgacgagcagcagaagaagcggctggaagcctttctcacccagaaagccaaggtcggcgaactcaaagacgatgacttcgaaaggatctcagagctgggcgcgggcaacggcggggtggtcaccaaagtccagcacagaccctcgggcctcatcatggccaggaagctgatccaccttgagatcaagccggccatccggaaccagatcatccgcgagctgcaggtcctgcacgaatgcaactcgccgtacatcgtgggcttctacggggccttctacagtgacggggagatcagcatttgcatggaacacatggacggcggctccctggaccaggtgctgaaagaggccaagaggattcccgaggagatcctggggaaagtcagcatcgcggttctccggggcttggcgtacctccgagagaagcaccagatcatgcaccgagatgtgaagccctccaacatcctcgtgaactctagaggggagatcaagctgtgtgacttcggggtgagcggccagctcatcgactccatggccaactccttcgtgggcacgcgctcctacatggctccggagcggttgcagggcacacattactcggtgcagtcggacatctggagcatgggcctgtccctggtggagctggccgtcggaaggtaccccatccccccgcccgacgccaaagagctggaggccatctttggccggcccgtggtcgacggggaagaaggagagcctcacagcatctcgcctcggccgaggccccccgggcgccccgtcagcggtcacgggatggatagccggcctgccatggccatctttgaactcctggactatattgtgaacgagccacctcctaagctgcccaacggtgtgttcacccccgacttccaggagtttgtcaataaatgcctcatcaagaacccagcggagcgggcggacctgaagatgctcacaaaccacaccttcatcaagcggtccgaggtggaagaagtggattttgccggctggttgtgtaaaaccctgcggctgaaccagcccggcacacccacgcgcaccgccgtgtgacagtggccgggctccctgcgtcccgctggtgacctgcccaccgtccctgtccatgccccgcccttccagctgaggacaggctggcgcctccacccaccctcctgcctcacccctgcggagagcaccgtggcggggcgacagcgcatgcaggaacgggggtctcctctcctgcccgtcctggccggggtgcctctggggacgggcgacgctgctgtgtgtggtctcagaggctctgcttccttaggttacaaaacaaaacagggagagaaaaagcaaaaaaaaaaaaaaaaaaaaaaaaaaaa

SEQ ID NO: 35 - protein, 400 aa, Homo sapiens [sequence omitted]

SEQ ID NO: 36 - DNA, 1902 bp, Homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 37 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 38 - DNA, 2005 bp, Homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgaggtgggccagtccccagcagcagtggggctgggggcaggggagcaggggggcacgtaggcatcccccatgccaggcctgagccttgctgtctctaccaccccagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 39 - protein, 357 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEVGQSPAAVGLGAGEQGGT

SEQ ID NO: 40 - DNA, 1770 bp, Homo sapiens
ctggcgcgcgcggccctgcgggtgacaggcaggcgggaaggggcggggcctcgggcggggccgccgtggggaggagggcggtgggaggggaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctggcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctgaaaaaaaaaaaaaaaaaaaaaaaaaaa

SEQ ID NO: 41 - protein, 335 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILALDLLDRMLTFNPNKRITVEEALAHPYLEQYYDPTDEPVAEEPFTFAMELDDLPKERLKELIFQETARFQPGVLEAP

SEQ ID NO: 42 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 43 - protein, 360 aa, Homo sapiens [sequence omitted]

SEQ ID NO: 44 - DNA, 1499 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggtacctggagtttaatacagtgagctctagcaagggaggcgctgccttttgtttctagaatattatgttcctcaaggtccattattttgtattcttttccaagctccttattggaaggtatttttttaaatttagaattaaaaattatttagaaagttacatataaa

SEQ ID NO: 45 - protein, 360 aa, Homo sapiens [sequence omitted]

(19) United States
(12) Patent Application Publication
(10) Pub. No.: US 2011/0262463 A1
(43) Pub. Date: Oct. 27, 2011

(54) SYNGR4 FOR TARGET GENES OF CANCER THERAPY AND DIAGNOSIS

(75) Inventors: Yusuke Nakamura, Tokyo (JP); Yataro Daigo, Tokyo (JP); Akira Togashi, Kanagawa (JP)

(73) Assignee: Oncotherapy Science ,Inc., Kawasaki-shi ,Kanagawa (JP)

(21) Appl. No.: 13/060,677

(22) PCT Filed: Aug. 24, 2009
(86) PCT No.: PCT/JP2009/004059

§ 371 Date: May 20, 2011

Related U.S. Application Data
(60) Provisional application No. 61/190,358, filed on Aug. 27, 2008.

(51) Int. Cl. A61K 39/395; C12N 15/63; C12Q 1/68; C40B 30/04; C12Q 1/48; C07K 16/18; A61K 31/713; A61P 35/00; C12Q 1/02; G01N 33/68; G01N 33/53; C07H 21/00

(52) U.S. Cl. 424/174.1; 436/501; 435/320.1; 435/6.13; 506/9; 536/24.31; 530/389.1; 536/24.5; 514/44 A; 435/29; 436/86; 435/15

(57) ABSTRACT

The present invention relates to the roles played by the SYNGR4 genes in lung cancer carcinogenesis and features a method for treating or preventing lung cancer by administering a double-stranded molecule against one or more of the SYNGR4 genes or a composition, vector or cell containing such a double stranded molecule and antibody. The present invention also features methods for diagnosing lung cancer or assessing/determining the prognosis of a patient with lung cancer, especially NSCLC or SCLC, using one or more over-expressed genes selected from among SYNGR4. To that end, SYNGR4 may serve as a novel serological biomarker for lung cancer. Also, disclosed are methods of identifying compounds for treating and preventing lung cancer, using as an index their effect on the over-expression of one or more of SYNGR4 in the lung cancer.

### CROSS-REFERENCE TO RELATED APPLICATIONS

The present application claims the benefit of U.S. Provisional Application No. 61/190,358, filed on Aug. 27, 2008, the entire disclosure of which is hereby incorporated herein by reference.

### TECHNICAL FIELD

The present invention relates to the field of biological science, more specifically to the field of cancer research, cancer diagnosis and cancer therapy. In particular, the present invention relates to methods for detecting and diagnosing lung cancer as well as methods for treating and preventing lung cancer. Moreover, the present invention relates to methods for screening agents for treating and/or preventing lung cancer.

### BACKGROUND ART

Lung cancer is one of the most common cancers and a leading cause of cancer deaths in the world (NPL 1: Shibuya K et al., BMC Cancer 2002, 2:37). Despite of the use of modern surgical techniques combined with various adjuvant treatment modalities, such as radiotherapy and chemotherapy, the overall 5-year survival rate of lung cancer patients still remains at about 20% (NPL 2: Naruke T, et al., Ann Thorac Surg 2001, 71(6): 1759-64). The recent development of molecularly targeted drugs such as gefitinib and bevacizumab provides hope, but fatal adverse events such as interstitial pneumonia by gefitinib or severe hemorrhage by bevacizumab have been reported. Therefore, the development of new agents targeting cancer specific molecules without adverse side effects is urgently needed (NPL 3: Ranson M, et al., J Clin Oncol 2002, 20: 2240-50; NPL 4: Inoue A, et al., Lancet 2003, 361: 137-9; NPL 5: Johnson D H, et al., J Clin Oncol 2004, 22: 2184-91). Oncoantigens, including some of cancer-testis antigens with oncogenic function, are an ideal therapeutic target. Cancer-testis antigens are defined to be proteins that are highly expressed in cancer cells but not in normal cells, except for cells in reproductive tissues, such as testis, ovary, and placenta. Because the cells from these tissues do not express MHC class I molecules, cancer-testis antigens are also considered to be a promising target for immunotherapy, such as cancer vaccines (NPL 6: Daigo Y, et al., Gen Thorac Cardiovasc Surg 2008, 56: 43-53).

Systematic analysis of expression levels of thousands of genes using a cDNA microarray technology is an effective approach for identifying molecules involved in pathways of carcinogenesis or those with efficacy to anticancer therapy; some of such genes or their gene products may be good target molecules for the development of novel therapies and/or cancer biomarkers. Previously, a genome-wide expression profile analysis of 101 clinical lung cancer samples was performed, coupled with enrichment of tumor cells by laser microdissection and then compared with the expression profile data of 31 normal human tissues (27 adult and 4 fetal organs) (NPL 6:Daigo Y, et al., Gen Thorac Cardiovasc Surg 2008, 56: 43-53; NPL 7:Kikuchi T, et al., Oncogene 2003, 22: 2192-205; NPL 8:Kakiuchi S, et al., Mol Cancer Res 2003, 1: 485-99; NPL 9:Kakiuchi S, et al., Hum Mol Genet 2004, 13: 3029-43; NPL 10:Kikuchi T, et al., Int J Oncol 2006, 28: 799-805; NPL 11:Taniwaki M, et al., Int J Oncol 2006, 29: 567-75).

In this invention, a screening system was established by a combination of tumor-tissue microarray analyses of clinical lung cancer materials and RNA interference techniques (NPL 12:Suzuki C, et al., Cancer Res 2003, 63: 7038-41; NPL 13:Ishikawa N, et al., Clin Cancer Res 2004, 10: 8363-70; NPL 14:Kato T, et al., Cancer Res 2005, 65: 5638-46; NPL 15:Furukawa C, et al., Cancer Res 2005, 65: 7102-10; NPL 16:Ishikawa N, et al., Cancer Res 2005, 65: 9176-84; NPL 17:Suzuki C, et al., Cancer Res 2005, 65: 11314-25; NPL 18:Ishikawa N, et al., Cancer Sci 2006, 97: 737-45; NPL 19:Takahashi K, et al., Cancer Res 2006, 66: 9408-19; NPL 20:Hayama S, et al., Cancer Res 2006, 66: 10339-48; NPL 21:Kato T, et al., Clin Cancer Res 2007, 13: 434-42; NPL 22:Suzuki C, et al., Mol Cancer Ther 2007, 6: 542-51; NPL 23:Yamabuki T, et al., Cancer Res 2007, 67: 2517-25; NPL 24:Hayama S, et al., Cancer Res 2007, 67: 4113-22 ; NPL 25:Kato T, et al., Cancer Res 2007, 67: 8544-53; NPL 26:Taniwaki M, et al., Clin Cancer Res 2007, 13: 6624-31; NPL 27:Ishikawa N, et al., Cancer Res 2007, 67: 11601-11; NPL 28:Mano Y, et al., Cancer Sci 2007, 98: 1902-13; NPL 29:Suda T, et al., Cancer Sci 2007, 98: 1803-8; NPL 30:Kato T, et al., Clin Cancer Res 2008, 14: 2363-70; NPL 31:Mizukami Y, et al., Cancer Sci 2008, 99: 1448-54). This systematic approach revealed that Synaptogyrin 4 (“SYNGR4”) is a novel cancer-testis antigen that is overexpressed commonly in primary lung cancers and is involved in cell invasion and growth/survival of cancer cells.

SYNGR4 is a 25 kD protein that was first described its chromosomal localization at 19q-arm glioma tumor suppressor region (NPL 32:Smith J S, et al., Genomics 2000, 64: 44-50). SYNGR4 is considered to be a new member of the synaptogyrin family. SYNGR1-3 are abundantly expressed on microvesicles in neuronal or non-neuronal cells (NPL 33:Kedra D, et al., Hum Genet 1998, 273: 2851-7; NPL 34:Janz R, et al., Neuron 1999, 24: 687-700; NPL 35:Zhao H, et al., Mol Biol Cell 2001, 12: 2275-89). The function of SYNGR1 and SYNGR2 (cellugyrin) has been well characterized. SYNGR1 and SYNGR2 have distinct expression profiles, in which SYNGR1 is mainly expressed in the central nervous system and SYNGR2 ubiquitously is expressed in various organs except brain (NPL 33:Kedra D, et al., Hum Genet 1998, 273: 2851-7). SYNGR1 protein localizes in synaptic vesicles of neurons and functionally influences the plasticity of neurons and endocytosis from the plasma membrane (NPL 34:Janz R, et al., Neuron 1999, 24: 687-700; NPL 35:Zhao H, et al., Mol Biol Cell 2001, 12: 2275-89). SYNGR2 protein is a component of synaptic-like microvesicles (SLMVs) which are ubiquitously observed in most types of cells (NPL 36:Belfort G M, et al., J Biol Chem 2003, 278: 47971-8; NPL 37:Belfort G M, et al., J Biol Chem 2005, 280: 7262-72). SYNGR2 is important for biogenesis of SLMVs by mobilizing the main component protein in SLMVs, synaptophysin, onto SLMVs (NPL 36:Belfort G M, et al., J Biol Chem 2003, 278: 47971-8; NPL 37:Belfort GM, et al., J Biol Chem 2005, 280: 7262-72). SYNGR3 protein exhibits the same expression profile of SYNGR1, but the exact function of SYNGR3 is unknown (NPL 38:Belizaire R, et al., J Comp Neurol 2004, 470: 266-81). The physiological and oncogenic function of SYNGR4 have not been well described.

### CITATION LIST

### Non Patent Literature

[NPL 1] Shibuya K et al., BMC Cancer 2002, 2:37

[NPL 2] Naruke T, et al., Ann Thorac Surg 2001, 71(6): 1759-64

[NPL 3] Ranson M, et al., J Clin Oncol 2002, 20: 2240-50

[NPL 4] Inoue A, et al., Lancet 2003, 361: 137-9

[NPL 5] Johnson D H, et al., J Clin Oncol 2004, 22: 2184-91

[NPL 6] Daigo Y, et al., Gen Thorac Cardiovasc Surg 2008, 56: 43-53

[NPL 7] Kikuchi T, et al., Oncogene 2003, 22: 2192-205

[NPL 8] Kakiuchi S, et al., Mol Cancer Res 2003, 1: 485-99

[NPL 9] Kakiuchi S, et al., Hum Mol Genet 2004, 13: 3029-43

[NPL 10] Kikuchi T, et al., Int J Oncol 2006, 28: 799-805

[NPL 11] Taniwaki M, et al., Int J Oncol 2006, 29: 567-75

[NPL 12] Suzuki C, et al., Cancer Res 2003, 63: 7038-41

[NPL 13] Ishikawa N, et al., Clin Cancer Res 2004, 10: 8363-70

[NPL 14] Kato T, et al., Cancer Res 2005, 65: 5638-46

[NPL 15] Furukawa C, et al., Cancer Res 2005, 65: 7102-10

[NPL 16] Ishikawa N, et al., Cancer Res 2005, 65: 9176-84

[NPL 17] Suzuki C, et al., Cancer Res 2005, 65: 11314-25

[NPL 18] Ishikawa N, et al., Cancer Sci 2006, 97: 737-45

[NPL 19] Takahashi K, et al., Cancer Res 2006, 66: 9408-19

[NPL 20] Hayama S, et al., Cancer Res 2006, 66: 10339-48

[NPL 21] Kato T, et al., Clin Cancer Res 2007, 13: 434-42

[NPL 22] Suzuki C, et al., Mol Cancer Ther 2007, 6: 542-51

[NPL 23] Yamabuki T, et al., Cancer Res 2007, 67: 2517-25

[NPL 24] Hayama S, et al., Cancer Res 2007, 67: 4113-22

[NPL 25] Kato T, et al., Cancer Res 2007, 67: 8544-53

[NPL 26] Taniwaki M, et al., Clin Cancer Res 2007, 13: 6624-31

[NPL 27] Ishikawa N, et al., Cancer Res 2007, 67: 11601-11

[NPL 28] Mano Y, et al., Cancer Sci 2007, 98: 1902-13

[NPL 29] Suda T, et al., Cancer Sci 2007, 98: 1803-8

[NPL 30] Kato T, et al., Clin Cancer Res 2008, 14: 2363-70

[NPL 31] Mizukami Y, et al., Cancer Sci 2008, 99: 1448-54

[NPL 32] Smith J S, et al., Genomics 2000, 64: 44-50

[NPL 33] Kedra D, et al., Hum Genet 1998, 273: 2851-7

[NPL 34] Janz R, et al., Neuron 1999, 24: 687-700

[NPL 35] Zhao H, et al., Mol Biol Cell 2001, 12: 2275-89

[NPL 36] Belfort GM, et al., J Biol Chem 2003, 278: 47971-8

[NPL 37] Belfort GM, et al., J Biol Chem 2005, 280: 7262-72

[NPL 38] Belizaire R, et al., J Comp Neurol 2004, 470: 266-81

### SUMMARY OF INVENTION

The present invention is based, in part, on the discovery of SYNGR4 as a member of the cancer-testis antigens, an ideal target for cancer vaccine therapy, and the role of SYNGR4 in pulmonary carcinogenesis and tumor progression. SYNGR4 is a useful prognostic biomarker and therapeutic target for lung cancer.

Accordingly, the present invention relates to SYNGR4, and its role in lung cancer carcinogenesis and other cancers that overexpress SYNGR4. As such, the present invention relates to compositions and methods for detecting, diagnosing, treating and/or preventing cancers that overexpress SYNGR4, e.g., lung cancer, particularly SCLC and NSCLC, as well as methods for screening for useful agents that bind and/or inhibit SYNGR4.

In particular, the present invention arises, in part, from the discovery that double-stranded molecules composed of specific sequences (in particular, SEQ ID NOs: 11, 12, 19 and 20) that inhibit the expression of SYNGR4 are effective for inhibiting cellular growth of cancers cells that overexpress SYNGR4, e.g., lung cancer cells. Specifically, small interfering RNAs (siRNAs) targeting SYNGR4 genes are provided by the present invention. These double-stranded molecules can be utilized in an isolated state or encoded in vectors and expressed from the vectors. Accordingly, it is an object of the present invention to provide such double stranded molecules as well as vectors and host cells expressing them that inhibit the expression of SYNGR4. In one aspect, the present invention provides methods for inhibiting cell growth and treating lung cancer by administering the double-stranded molecules or vectors of the present invention to a subject in need thereof. Such methods encompass administering to a subject a composition composed of one or more of the double-stranded molecules or vectors.

In another aspect, the present invention provides compositions for treating a lung cancer containing at least one of the double-stranded molecules or vectors of the present invention that are effective in inhibiting the expression of SYNGR4.

In yet another aspect, the present invention provides a method of diagnosing or determining a predisposition to lung cancer in a subject by determining an expression level of SYNGR4 in a patient derived biological sample. An increase in the expression level of SYNGR4 as compared to a normal control level of SYNGR4 indicates that the subject suffers from or is at risk of developing lung cancer.

Moreover, the present invention relates to the discovery that a high expression level of SYNGR4 correlates to poor survival rate in a cancer patient, particularly in a lung cancer patient. Therefore, the present invention provides a method for assessing or determining the prognosis of a patient with cancer, e.g., lung cancer, which method includes the steps of detecting the expression level of SYNGR4, comparing it to a pre-determined reference expression level and determining the prognosis of the patient from the difference there between.

In a further aspect, the present invention provides a method of screening for a compound for treating and/or preventing a cancer promoted or caused or promoted in part by overexpression of SYNGR4, e.g., lung cancer. Such a compound would bind with the SYNGR4 gene, reduce the biological activity of SYNGR4, inhibit the interaction between SYNGR4 and other proteins or reduce the expression of the SYNGR4 gene or a reporter gene that was controlled by the transcription initiation region of the SYNGR4 gene.

In one aspect, present invention provides a method of treating or preventing a cancer promoted or caused or promoted in part by overexpression of SYNGR4, e.g., lung cancer by administering an antibody that binds to and/or inhibits the activity of the SYNGR4 protein.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of preferred embodiments, and not restrictive of the invention or other alternate embodiments of the invention.

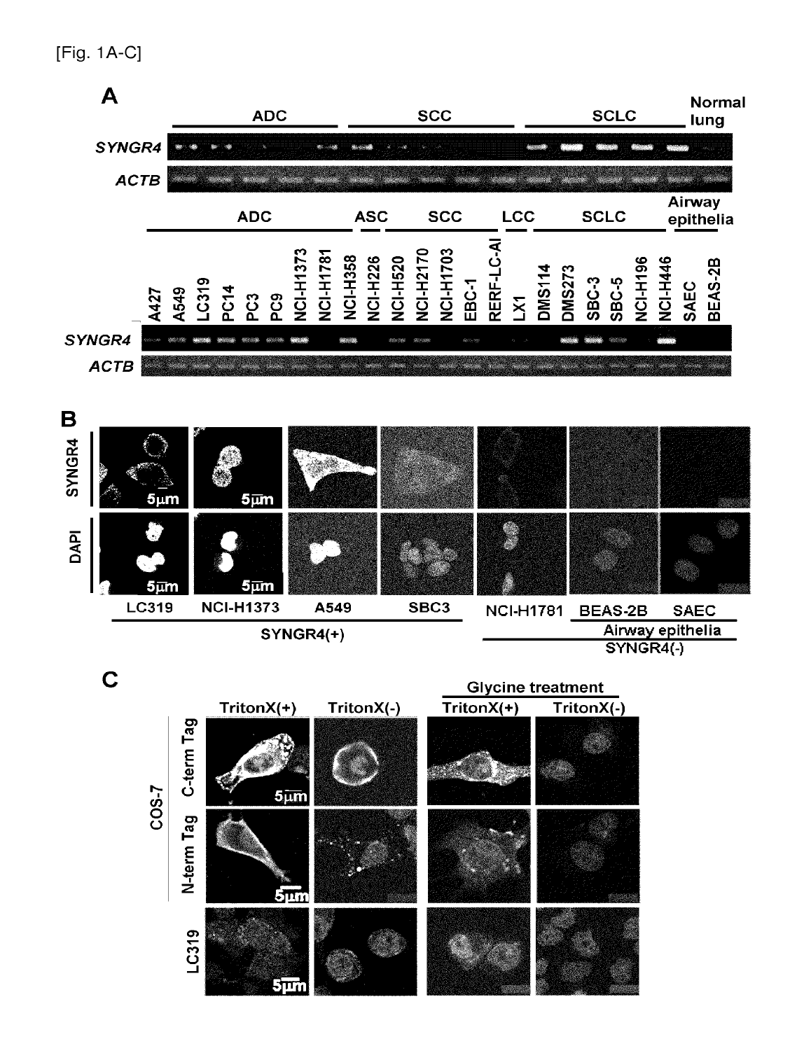

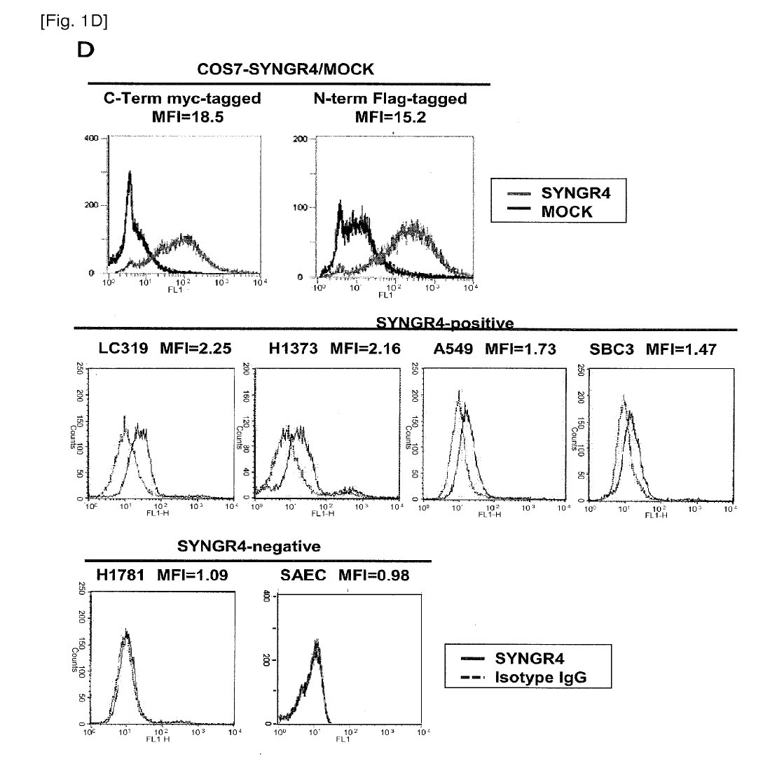

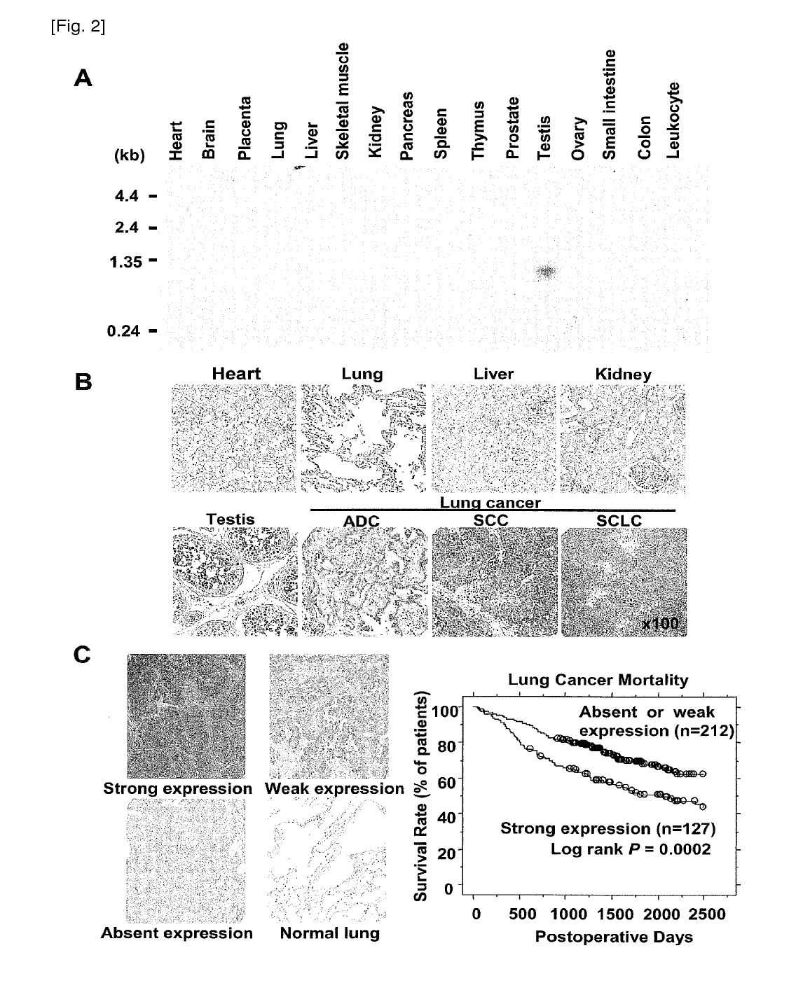

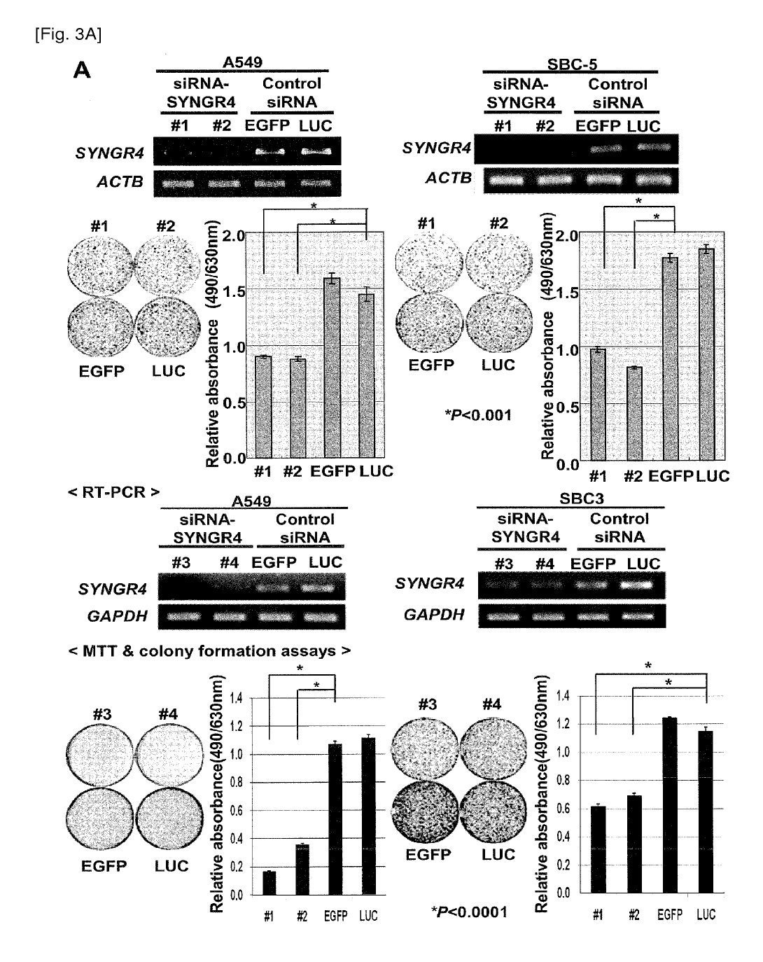

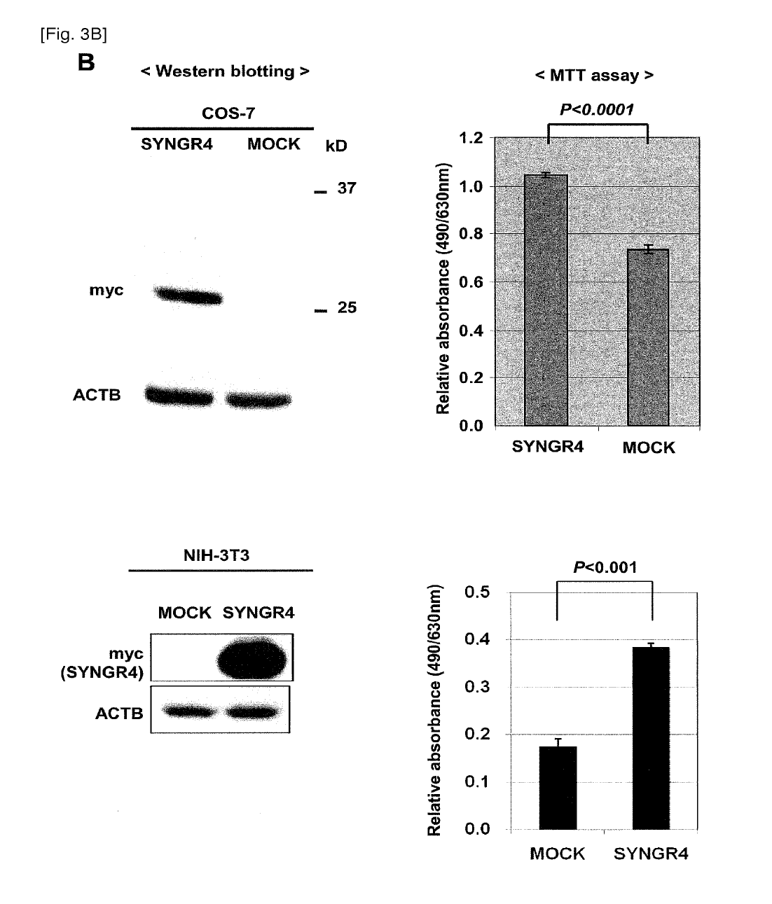

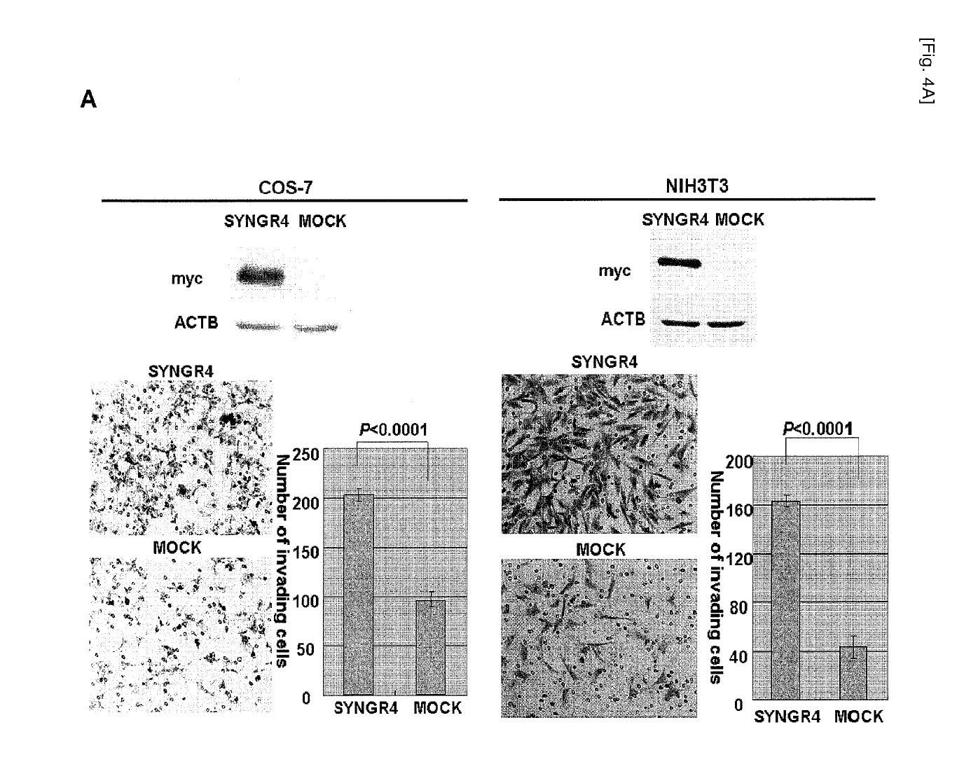

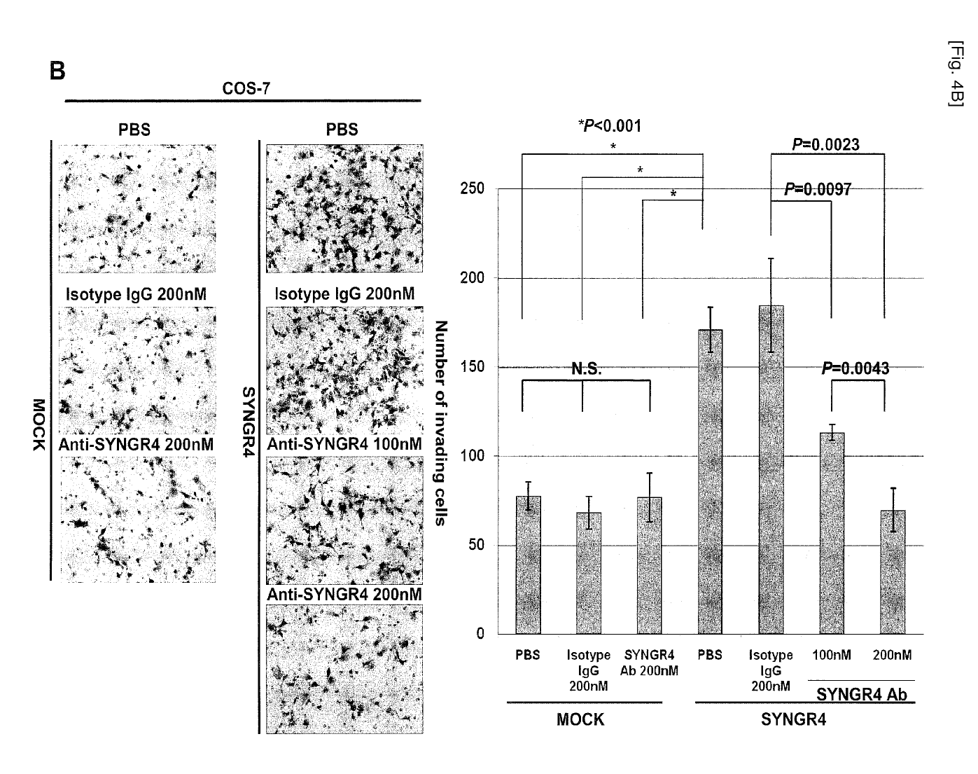

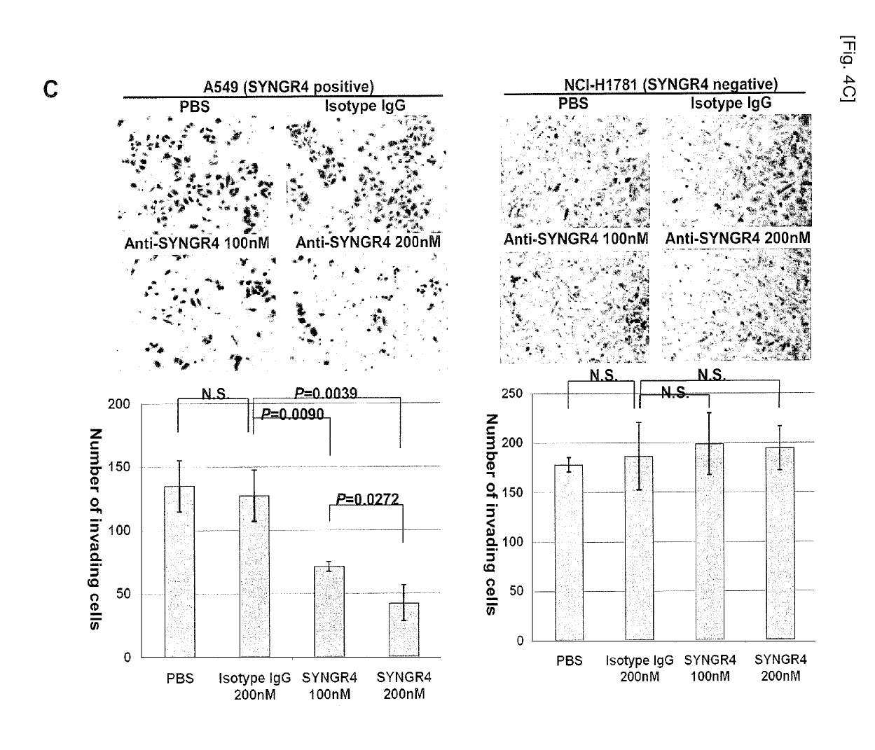

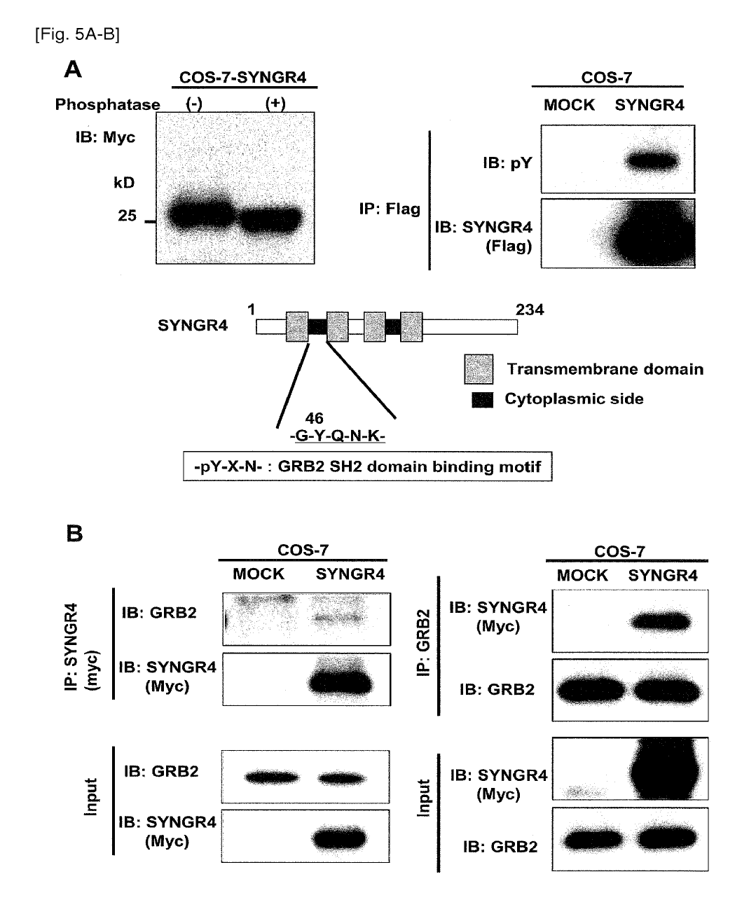

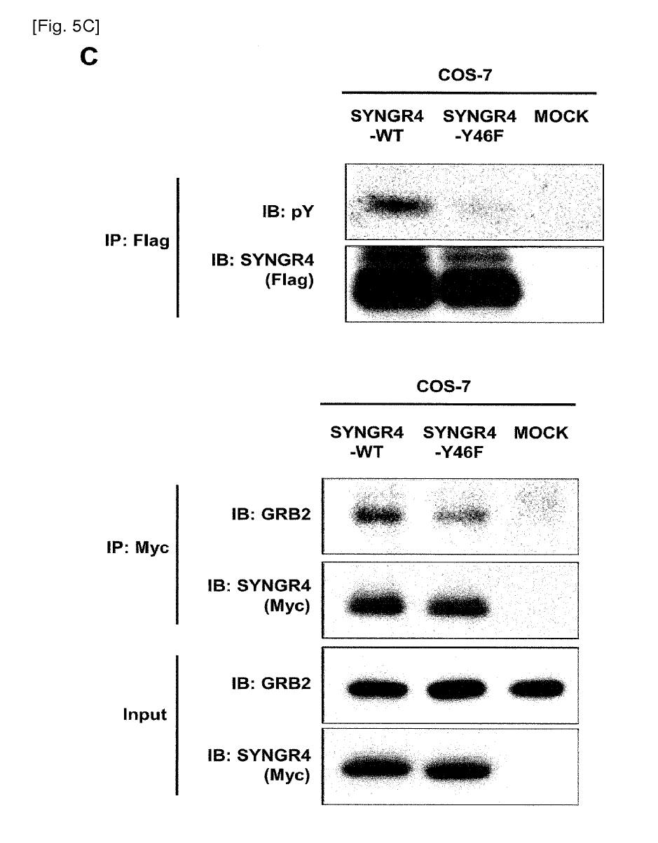

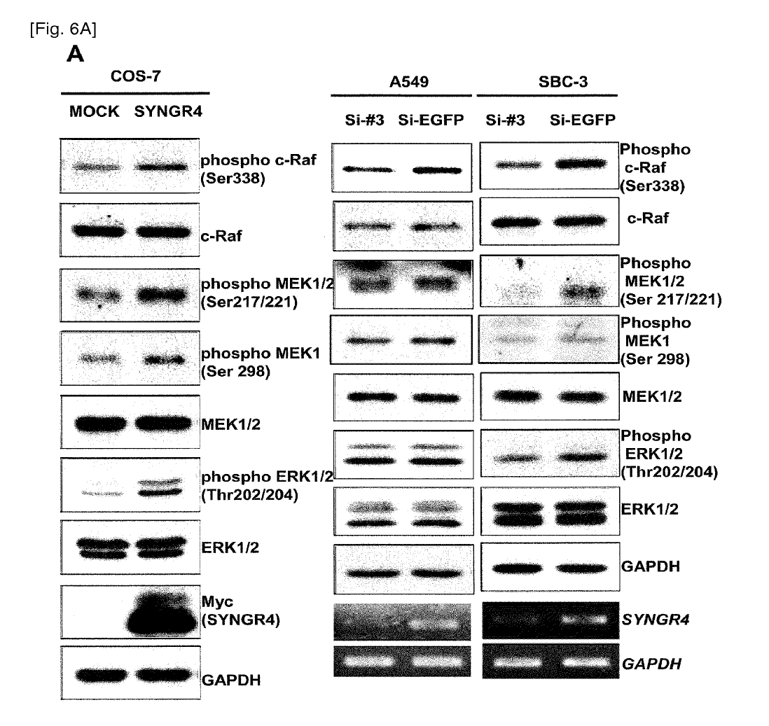

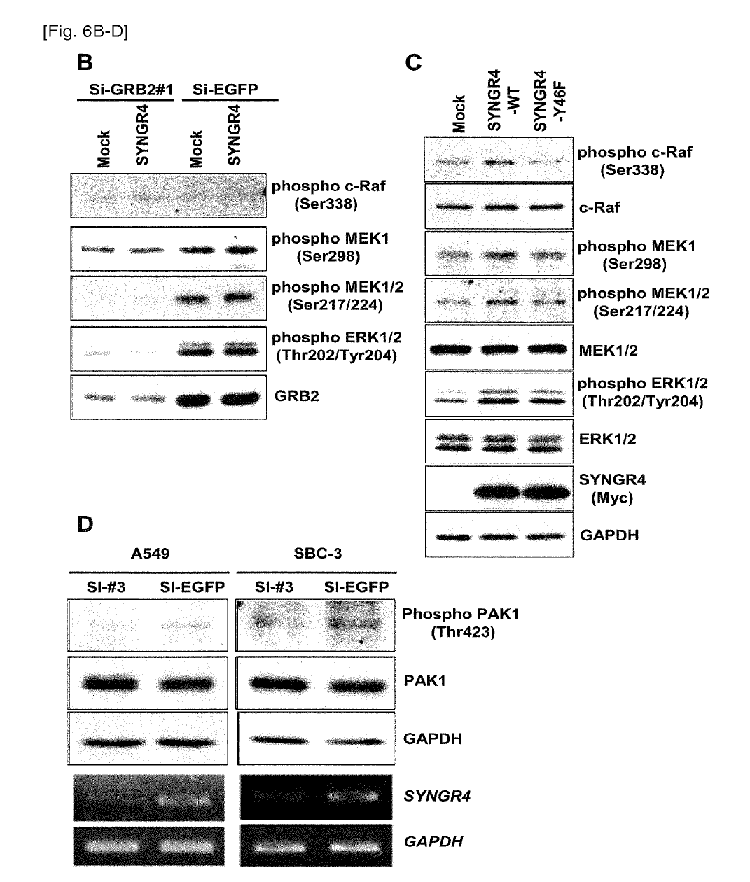

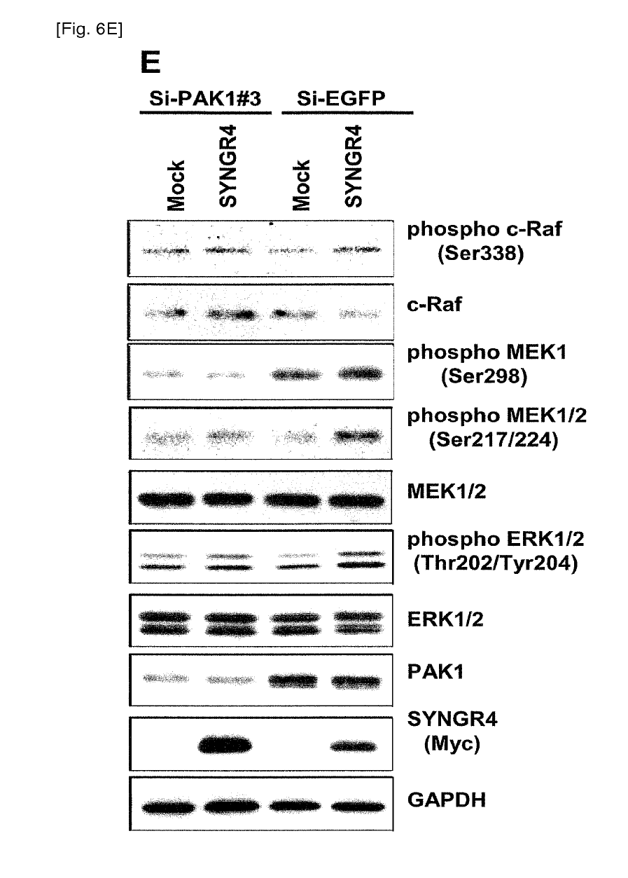

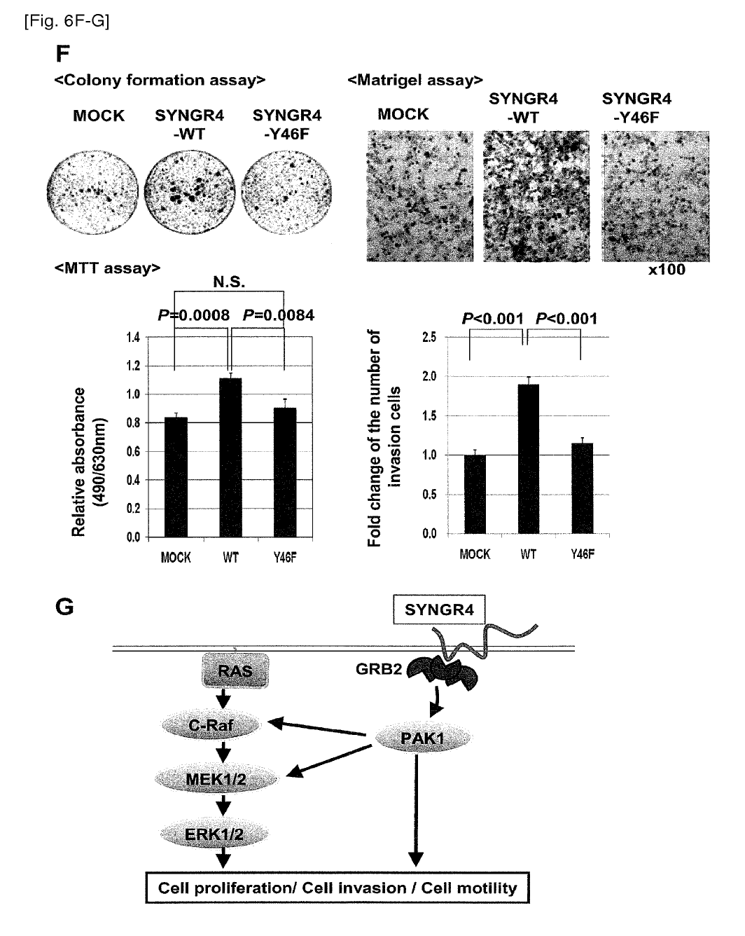

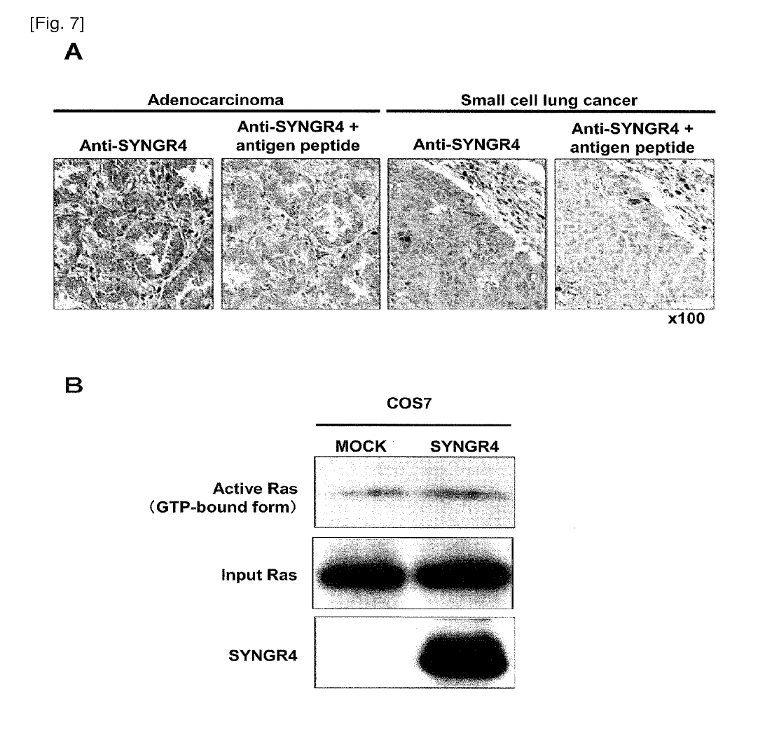

### BRIEF DESCRIPTION OF DRAWINGS

[FIG. 1A-C] Analysis of SYNGR4 expression in tumor tissues, cell lines and normal tissue. A, Expression of SYNGR4 in 15 clinical lung cancer and normal lung tissue samples (top panels) [lung adenocarcinoma (ADC), lung squamous cell carcinoma (SCC) and small cell lung carcinoma (SCLC); top] and 22 lung cancer cell lines (bottom panels) detected by semiquantitative RT-PCR analysis. B, Expression and sub-cellular localization of endogenous SYNGR4 protein in SYNGR4-positive and −negative lung cancer cell lines, and bronchial epithelial cells. SYNGR4 was stained mainly at the cytoplasm and weakly on cell surface with granular appearance in NCI-H1373, LC319, A549, and SBC3 cells, whereas no staining was observed in NCI-H1781 and bronchial epithelia derived BEAS-2B and SAEC cell lines. C, Detection of cell surface SYNGR4 by immunocytochemistry using anti-myc antibody in COS-7 cells transfected with C-terminal myc/His-tagged SYNGR4 and anti-Flag antibody in COS-7 cells transfected with N-terminal 3× Flag-tagged SYNGR4. SYNGR4 staining on cell surface was observed with and without Triton-X treatment, whereas SYNGR4 staining was disappeared by removing cell surface antibody with acid glycine, indicating that C-terminus and N-terminus of SYNGR4 is outside of cell membrane.

[FIG. 1D] D, Detection of cell surface SYNGR4 in lung cancer cell lines by flow cytometry. SYNGR4 was detected on cell surface of SYNGR4 expressing cell lines.

[FIG. 2] Expression of SYNGR4 in normal tissues and lung cancers, and its association with poor prognosis for NSCLC patients. A, Northern blot analysis of the SYNGR4 transcript in 16 normal adult human tissues. A strong signal was observed in testis. B, Comparison of SYNGR4 protein expression between normal tissues and lung cancers by immunohistochemistry. C, Examples for strong, weak, and absent SYNGR4 expression in lung cancer tissues and a normal tissue (left photos). Original magnification, ×100. Kaplan-Meier analysis of survival of patients with NSCLC (P=0.0002 by log-rank test) according to expression levels of SYNGR4 (right panel).

[FIG. 3A] Growth promoting effect of SYNGR4. A, Inhibition of growth of lung cancer cells by siRNAs against SYNGR4. Upper panels, Gene knockdown effect on SYNGR4 expression in A549 cells (left) and SBC-5 cells (right) by si-SYNGR4s (#1 and #2) and control siRNAs (si-EGFP/enhanced green fluorescent protein gene, si-LUC/Luciferase), analyzed by semiquantitative RT-PCR. Bottom panels, Colony formation and MTT assays of A549 and SBC-5 cells transfected with si-SYNGR4s or control siRNAs. Columns, relative absorbance of triplicate assays; bars, SD.

[FIG. 3B] B, Promotion of cell proliferation in COS-7 or NTH-3T3 cells exogenously overexpressing SYNGR4. Upper panel, Detection of transient SYNGR4 expression by western blotting. Bottom panel, MTT assays of COS-7 cells 96 hours after transfection of SYNGR4-expressing vector. Columns, relative absorbance of triplicate assays; bars, SD.

[FIG. 4A] Cellular invasive effect of SYNGR4. A, Promotion of cell invasion in mammalian cells exogenously overexpressing SYNGR4. Top panels, Transient expression of SYNGR4 protein in COS-7 (left) and NIH3T3 (right) cells, detected by western-blotting. Bottom panels, Assay demonstrating the invasive nature of COS-7 and NTH3T3 cells in Matrigel matrix after transfection with expression plasmids for human SYNGR4. Giemsa staining (× 200; left bottom), and graph panels representing the number of cells migrating through the Matrigel-coated filters (right bottom). Bars, SD. Assays were performed three times and in triplicate wells.

[FIG. 4B] B, Inhibitory effect of anti-SYNGR4 antibody on the cell invasive activity in exogenously SYNGR4 expressing COS-7 cells. Left panels, Microscopic evaluation of invaded COS-7 cells treated with anti-SYNGR4 antibody or isotype IgG, or PBS. Right panel, The number of COS-7 cells migrating through the Matrigel-coated filters; bars, SD. Assays were done for three times and in triplicate wells.

[FIG. 4C] C, Inhibitory effect of anti-SYNGR4 antibody on cell invasive activity in lung cancer cells. Top panels, Microscopic evaluation of invaded A549 cells (left) and NCI-H1781 cells (right) treated with anti-SYNGR4 antibody, isotype IgG, or PBS. Bottom panels, The number of invading cancer cells through the Matrigel-coated filters; bars, SD. Assays were done thrice in triplicate wells.

[FIG. 5A-B] Interaction of SYNGR4 with GRB2. A, Identification of phosphorylation of SYNGR4. Left top panels, phosphatase treatment of exogenous SYNGR4 protein in COS-7 cells. Shifted band in phosphatase-treated samples indicates that SYNGR4 is phosphorylated. Left bottom panels, immunoblot of immunoprecipitants of lysates from COS-7 cells that was exogenously expressed SYNGR4 or those transfected with Mock vector by anti-phosphotyrosine antibody. Right panel, Protein sequence of SYNGR4 protein. Number indicates the amino acid from N-terminus. B, Confirmation of exogenous SYNGR4 binding with GRB2 by immunoprecipitation (IP) in mammalian cells. Left panel, COS-7 cells were transfected with mock or SYNGR4 and subjected to IP-myc by anti-myc antibody, followed by immunoblotting (IB) with anti-GRB2 antibody. Immunoblotting using cell lysates being not precipitated (input) was performed at the same time. The re-immunoblotting with anti-myc antibody was performed to confirm the immunoprecipitation of SYNGR4. Right panel, COS-7 cells were transfected with Mock or SYNGR4 and carried out IP-GRB2 by anti-GRB2 antibody, followed by immunoblotting with anti-myc antibody. The re-immunoblotting with anti-GRB2 antibody was performed to confirm the immunoprecipitation of GRB2.

[FIG. 5C] C, Tyrosine-46 in SYNGR4 was phosphorylated and important for interaction with GRB2 by replaced of tyrosine-46 with phenylalanine in SYNGR4 (SYNGR4-Y46F). Left panels, COS-7 cells were transfected with Mock or wild type (WT) of SYNGR4 or SYNGR4-Y46F and IP-Flag with anti-Flag antibody, followed by immunoblotting (IB) with anti-phosphotyrosine antibody. Right panel, COS-7 cells were transfected with Mock or SYNGR4-WT or Y46F and subjected to IP-myd with anti-myc antibody, followed by immunoblotting with anti-GRB2 antibody. The re-immunoblotting with anti-myc antibody was performed to confirm the immunoprecipitation of SYNGR4.

[FIG. 6A] Activation of MAPK signaling through its upstream SYNGR4-dependent GRB2-PAK1 pathway. A, Status of MAPK signaling molecules phosphorylation by SYNGR4 or siRNA for SYNGR4 transfection in mammalian cells. Left panel, COS-7 cells were transfected with Mock or SYNGR4 and 24 hours after transfection cell lysates were subjected to immunoblotting with anti-phospho c-RAF (Ser338), anti-c-RAF, anti-phospho MEK1/2 (Ser217/221), anti-phospho MEK1 (Ser298), anti-phospho ERK (Thr202/Tyr204), anti-ERK, anti-myc, and anti-GAPDH antibodies. Right panels, A549 and SBC-3 cells were transfected with siRNA for SYNGR4 or control siRNA (EGFP) and 24 hours after transfection cell lysates were carried out immunoblotting with anti-phospho c-RAF (Ser338), anti-c-RAF, anti-phospho MEK1/2 (Ser217/221), anti-phospho MEK1 (Ser298), anti-phospho ERK (Thr202/Tyr204), anti-ERK, and anti-GAPDH antibodies. Cell total RNA is also acquired and carried out reverse-transcription reaction, followed by PCR reaction to evaluate knockdown of SYNGR4 transcription. GAPDH transcription was used as internal control.

[FIG. 6B-D] B, Effect of enhancing MAPK signaling by SYNGR4 is mediated via GRB2. COS-7 cells were transfected with siRNA for GRB2 or control siRNA (EGFP) and 4 hours after transfection cells were transfected with mock or SYNGR4. 24 hours after second transfection cell lysates were subsequently subjected to immunoblotting with anti-phospho c-RAF (Ser338), anti-c-RAF, anti-phospho MEK1/2 (Ser217/221), anti-phospho MEK1 (Ser298), anti-phospho ERK (Thr202/Tyr204), anti-ERK, anti-GRB2, anti-myc, and anti-GAPDH antibodies. C, Impaired function of enhancing MAPK signaling in mutant-SYNGR4 expressing COS-7 cells. COS-7 cell were transfected with mock, wild type SYNGR4 (WT), or mutant SYNGR4 (Y46F) and 24 hours after transfection cell lysates were subjected to immunoblotting with anti-phospho c-RAF (Ser338), anti-c-RAF, anti-phospho MEK1/2 (Ser217/221), anti-phospho MEK1 (Ser298), anti-phospho ERK (Thr202/Tyr204), anti-ERK, anti-myc, and anti-GAPDH antibodies. D, Effect of PAK1 phosphorylation by SYNGR4 transfection in lung cancer cells. A549 and SBC-3 cells were transfected with siRNA for SYNGR4 or control siRNA (EGFP) and 24 hours after transfection cell lysates were carried out immunoblotting with anti-phospho PAK1 (Thr423), PAK1, and GAPDH antibodies. Cell total RNA is also acquired and carried out reverse-transcription reaction, followed by PCR reaction to evaluate knockdown of SYNGR4 transcription. GAPDH transcription was used as internal control.

[FIG. 6E] E, Effect of enhancing MAPK signaling by SYNGR4 is mediated via PAK1. COS-7 cells were transfected with siRNA for PAK1 or control siRNA (EGFP) and 4 hours after transfection cells were transfected with mock or SYNGR4. 24 hours after second transfection cell lysates were subsequently subjected to immunoblotting with anti-phospho c-RAF (Ser338), anti-c-RAF, anti-phospho MEK1/2 (Ser217/221), anti-phospho MEK1 (Ser298), anti-phospho ERK (Thr202/Tyr204), anti-ERK, anti-PAK1, anti-myc, and anti-GAPDH antibodies.

[FIG. 6F-G] F, Impaired function of promoting cell growth and invasion in mutant SYNGR4 expressing COS-7 cells. Left panels, COS-7 cells were transfected with mock, wild type SYNGR4 (WT), or mutant SYNGR4 (Y46F) and 120 hours after colony formation (Top) and MTT (bottom) assays were performed. Columns, relative absorbance of triplicate assays; bars, SD. Right panels, COS-7 cells were transfected with mock, SYNGR4-WT, or SYNGR4-Y46F and were applied and incubated in Matrigel Invasion chamber for 22 hours, followed by counting cells migrating through the Matrigel-coated filters. Giemsa staining (× 200; right top), and graph panels representing the number of migrating cells (right bottom). Bars, SD. Assays were performed three times and in triplicate wells. G, Possible interacting cascade related to SYNGR4.

[FIG. 7] Supplementary figures. A, Immunohistochemistry of lung cancer tissues with or without pre-incubating antigen peptide for SYNGR4 antibody. B, RAF1 pull-down assay for detecting GTP-bound RAS. COS-7 cells were transfected with mock or SYNGR4 expressing plasmids and 24 hours after cell lysates were subjected to incubate with recombinant active RAF1 conjugated beads, followed by immunoblotting with anti-RAS antibody.

### DESCRIPTION OF EMBODIMENTS

Definition

The words “a”, “an”, and “the” as used herein mean “at least one” unless otherwise specifically indicated.

As used herein, the term “biological sample” refers to a whole organism or a subset of its tissues (e.g., lung tissue), cells or component parts (e.g., body fluids, including but not limited to blood, serum, plasma, mucus, lymphatic fluid, synovial fluid, cerebrospinal fluid, saliva, sputum, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The term “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

SYNGR4 Gene or SYNGR4 Protein

The nucleic acid and polypeptide sequences of genes in present invention are shown in the following numbers, but not limited to those;

SYNGR4: SEQ ID NO: 13 and 14.

Furthermore, the sequence data is also available via the following accession number:

SYNGR4: NM_{—}012451.

The present invention first discloses the SYNGR4 expression could promote progression of lung tumors by stimulating cell proliferation/survival and metastasis through activating a new GRB2/PAK1/MAPK signaling pathway.

GRB2 Gene or GRB2 Protein

The nucleic acid and polypeptide sequences of genes in present invention are shown in the following numbers, but not limited to those;

GRB2: SEQ ID NO: 22 to 25.

Furthermore, the sequence data are also available via the following accession numbers:

GRB2: NM_{—}002086 and NM_{—}203506.

The protein encoded by this gene binds the epidermal growth factor receptor and contains one SH2 domain and two SH3 domains. Its two SH3 domains direct complex formation with proline-rich regions of other proteins, and its SH2 domain binds tyrosine phosphorylated sequences. This gene is similar to the Sem5 gene of C.elegans, which is involved in the signal transduction pathway. Two alternatively spliced transcript variants encoding different isoforms have been found for this gene. The variant (1) (NM_{—}002086) represents the longer transcript and encodes the longer isoform.

PAK1 Gene or PAK1 Protein

The nucleic acid and polypeptide sequence of the gene in the present invention are shown in the following numbers, but not limited to those;

PAK1: SEQ ID NO: 26 to 29.

Furthermore, the sequence data are also available via following accession numbers:

PAK1: NM_{—}001128620 and NM_{—}002576.

PAK proteins are critical effectors that link RhoGTPases to cytoskeleton reorganization and nuclear signaling. PAK proteins, a family of serine/threonine p21-activating kinases, include PAK1, PAK2, PAK3 and PAK4. These proteins serve as targets for the small GTP binding proteins Cdc42 and Rac and have been implicated in a wide range of biological activities. PAK1 regulates cell motility and morphology. Alternatively spliced transcript variants encoding different isoforms have been found for this gene. The variant (1)(NM_{—}001128620) encodes the longer isoform.

c-Raf Gene or c-Raf Protein

The nucleic acid and polypeptide sequences of genes in present invention are shown in the following numbers, but not limited to those;

c-Raf: SEQ ID NO: 30 to 31;

Furthermore, the sequence data are also available via following accession numbers.

c-Raf: NM_{—}002880;

This gene is the cellular homolog of viral raf gene (v-raf). The encoded protein is a MAP kinase kinase kinase (MAP3K), which functions downstream of the Ras family of membrane associated GTPases to which it binds directly. Once activated, the cellular RAF1 protein can phosphoryl ate to activate the dual specificity protein kinases MEK1 and MEK2, which in turn phosphorylate to activate the serine/threonine specific protein kinases, ERK1 and ERK2. Activated ERKs are pleiotropic effectors of cell physiology and play an important role in the control of gene expression involved in the cell division cycle, apoptosis, cell differentiation and cell migration. Mutations in this gene are associated with Noonan syndrome 5 and LEOPARD syndrome 2.

Gene or Protein of MEK1/2

The nucleic acid and polypeptide sequences of genes in present invention are shown in the following numbers, but not limited to those;

MEK1: SEQ ID NO: 32 and 33, MEK2: SEQ ID NO: 34 and 35.

Furthermore, the sequence data are also available via following accession numbers:

MEK1: NM_{—}002755, MEK2: NM_{—}030662.

MEK1 is a member of the dual specificity protein kinase family, which acts as a mitogen-activated protein (MAP) kinase kinase. MAP kinases, also known as extra-cellular signal-regulated kinases (ERKs), act as an integration point for multiple bio-chemical signals. This protein kinase lies upstream of MAP kinases and stimulates the enzymatic activity of MAP kinases upon wide variety of extra- and intracellular signals. As an essential component of MAP kinase signal transduction pathway, this kinase is involved in many cellular processes such as proliferation, differentiation, transcription regulation and development. MEK2 is a dual specificity protein kinase that belongs to the MAP kinase kinase family. This kinase is known to play a critical role in mitogen growth factor signal transduction. It phosphorylates and thus activates MAPK1/ERK2 and MAPK2/ERK3. The activation of this kinase itself is dependent on the Ser/Thr phosphorylation by MAP kinase kinase kinases. Mutations in this gene cause cardiofaciocutaneous syndrome retardation, and distinctive facial features similar to those found in Noonan syndrome. The inhibition or degradation of this kinase is also found to be involved in the pathogenesis of Yersinia and anthrax. A pseudogene, which is located on chromosome 7, has been identified for this gene.

Gene or Protein of ERK1/2

The nucleic acid and polypeptide sequences of genes in present invention are shown in the following numbers, but not limited to those;

ERK1: SEQ ID NO: 36 to 41, ERK2: SEQ ID NO: 42 to 45.

Furthermore, the sequence data are also available via following accession numbers:

ERK1: NM_{—}002746, NM_{—}001040056, NM_{—}001109891, ERK2: NM_{—}002745, NM_{—}138957.

ERK1 is a member of the MAP kinase family. MAP kinases, also known as extra-cellular signal-regulated kinases (ERKs), act in a signaling cascade that regulates various cellular processes such as proliferation, differentiation, and cell cycle progression in response to a variety of extracellular signals. This kinase is activated by upstream kinases, resulting in its translocation to the nucleus where it phosphorylates nuclear targets. Alternatively spliced transcript variants encoding different protein isoforms have been described (NM_{—}002746, NM_{—}001040056, NM_{—}001109891). The variant (1) (NM_{—}002746) represents the most common transcript and encodes isoform 1. ERK2 is a member of the MAP kinase family. MAP kinases, also known as extra-cellular signal-regulated kinases (ERKs), act as an integration point for multiple bio-chemical signals, and are involved in a wide variety of cellular processes such as pro-liferation, differentiation, transcription regulation and development. The activation of this kinase requires its phosphorylation by upstream kinases. Upon activation, this kinase translocates to the nucleus of the stimulated cells, where it phosphorylates nuclear targets. Two alternatively spliced transcript variants encoding the same protein, but differing in the UTRs, have been reported for this gene. This variant (1) (NM_{—}002745) represents the longer transcript. Both variants 1 (NM_{—}002745) and 2 (NM_{—}138957) encode the same protein.

According to an aspect of the present invention, functional equivalents are also considered to be above “polypeptides”. Herein, a “functional equivalent” of a protein is a polypeptide that has a biological activity equivalent to the protein. Namely, any polypeptide that retains the biological ability may be used as such a functional equivalent in the present invention. Such functional equivalents include those wherein one or more amino acids are substituted, deleted, added, or inserted to the natural occurring amino acid sequence of the protein. Alternatively, the polypeptide may be composed an amino acid sequence having at least about 80% homology (also referred to as sequence identity) to the sequence of the respective protein, more preferably at least about 90%, 93%, 95%, 97%, 99% sequence identity to a reference sequence, e.g., a SYNGR4 polypeptide, e.g., SEQ ID NO:14, as determined using a known sequence comparison algorithm, e.g., BLAST or ALIGN, set to default settings. In other embodiments, the polypeptide can be encoded by a polynucleotide that hybridizes under stringent conditions to the natural occurring nucleotide sequence of the gene. In some embodiments, the polypeptide is encoded by a polynucleotide that shares at least about 90%, 93%, 95%, 97%, 99% sequence identity to a reference sequence, e.g., a SYNGR4 polynucleotide, e.g., SEQ ID NO:13, as determined using a known sequence comparison algorithm.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a functional equivalent to that of the human protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42 degrees C., or, 5×SSC, 1% SDS, incubating at 65 degrees C., with wash in 0.2×SSC, and 0.1% SDS at 50 degrees C.

In the context of the present invention, a condition of hybridization for isolating a DNA encoding a polypeptide functionally equivalent to the above human protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42 degrees C., 2×SSC, 0.1% SDS, preferably 50 degrees C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. In the present invention, the inventors demonstrated that the strong SYNGR4 expression was associated with poorer clinical outcome for NSCLC patients, inhibition of endogenous expression of SYNGR4 by siRNA resulted in marked reduction of viability of lung cancer cells, and exogenous expression of SYNGR4 enhanced the cell growth and cellular migration/invasive activity in mammalian cells. Furthermore, it was revealed that Tyr46 in SYNGR4 was phosphorylated and important for binding with GRB2 and also for activating GRB2/PAK1/MAPK signaling pathway. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins 1984).

Such conservatively modified polypeptides are included in the present protein. However, the present invention is not restricted thereto and the protein includes non-conservative modifications, so long as at least one biological activity of the protein is retained. Furthermore, the modified proteins do not exclude polymorphic variants, interspecies homologues, and those encoded by alleles of these proteins.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

Antibody

The terms “antibody” as used herein is intended to include immunoglobulins and fragments thereof which are specifically reactive to the designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. An “antibody” indicates all classes (e.g. IgA, IgD, IgE, IgG and IgM).

An antibody that specifically binds to SYNGR4 is useful for inhibiting lung cancer cell proliferation and invasive activity (FIGS. 4A-C and FIG. 6F).

Therefore the antibodies of the present invention find use for treating lung cancer. These antibodies can be produced by known methods. Exemplary techniques for the production of the antibodies used in accordance with the present invention are known in the art and described herein.

The present invention uses antibodies against SYNGR4 for the treatment and prevention of lung cancer. These antibodies will be provided by known methods.

Exemplary techniques for the production of the antibodies used in accordance with the present invention are described.

(i) Polyclonal Antibodies:

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimi de ester (conjugation through cysteine residues), N-hydroxysuccinimide (through lysine residues), glutaraldehyde, succinic anhydride, SOCl₂, or R′N═C═NR, where R and R are different alkyl groups. Animals are immunized against the antigen, e.g., SYNGR4, immunogenic conjugates, or derivatives by combining, e.g. 100 micro-g or 5 micro-g of the protein or conjugate (for rabbits or mice, respectively) with 3 volumes of Freund's complete adjuvant and injecting the solution intradermally at multiple sites. One month later the animals are boosted with 1/5 to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent.

Conjugates also can be made in recombinant cell culture as protein fusions. Also, aggregating agents such as alum are suitably used to enhance the immune response.

(ii) Monoclonal Antibodies:

Monoclonal antibodies are obtained from a population of substantially homogeneous antibodies, i.e., the individual antibodies including the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler G & Milstein C. Nature. 1975 Aug. 7; 256(5517):495-7, or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor D, et al., J Immunol. 1984 December; 133(6):3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the 30 Scatchard analysis of Munson P J & Rodbard D. Anal Biochem. 1980 Sep. 1; 107(1):220-39.

After hybridoma cells are identified that produce antibodies of the desired specificity, affinity, and/or activity, the clones may be subcloned by limiting dilution procedures and grown by standard methods (Goding, Monoclonal Antibodies : Principles and Practice, pp. 59-103 (Academic Press, 1986)). Suitable culture media for this purpose include, for example, D-MEM or RPML-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra A. Curr Opin Immunol. 1993 April; 5(2):256-62 and Pluckthun A. Immunol Rev. 1992 December; 130:151-88.

Another method of generating specific antibodies, or antibody fragments, reactive against a SYNGR4 is to screen expression libraries encoding immunoglobulin genes, or portions thereof, expressed in bacteria with SYNGR4. For example, complete Fab fragments, VH regions and Fv regions can be expressed in bacteria using phage expression libraries. See for example, Ward E S, et al., Nature. 1989 Oct. 12; 341(6242):544-6; Huse W D, et al., Science. 1989 Dec. 8; 246(4935):1275-81; and McCafferty J, et al., Nature. 1990 Dec. 6; 348(6301):552-4. Screening such libraries with, SYNGR4 peptide, can identify immunoglobulin fragments reactive with SYNGR4. Alternatively, the SCID-hu-mouse (available from Genpharm) can be used to produce antibodies or fragments thereof.

In a further embodiment, antibodies or antibody fragments can be isolated from antibody phage libraries generated using the techniques described in McCafferty J, et al., Nature. 1990 Dec. 6; 348(6301):552-4; Clarkson T, et al., Nature. 1991 Aug. 15; 352(6336):624-8; and Marks J D, et al., J MoL BioL, 222: 581-597 (1991) J Mol Biol. 1991 Dec. 5; 222(3):581-97 describe the isolation of murine and human antibodies, respectively, using phage libraries. Subsequent publications describe the production of high affinity (nM range) human antibodies by chain shuffling (Marks J D, et al., Biotechnology (NY). 1992 July; 10(7):779-83), as well as combinatorial infection and in vivo recombination as a strategy for constructing very large phage libraries (Waterhouse P, et al., Nucleic Acids Res. 1993 May 11; 21(9):2265-6). Thus, these techniques are viable alternatives to traditional monoclonal antibody hybridoma techniques for isolation of monoclonal antibodies.

The DNA also may be modified, for example, by substituting the coding sequence for human heavy-and light-chain constant domains in place of the homologous murine sequences (U.S. Pat. No. 4,816,567; Morrison S L, et al., Proc Natl Acad Sci USA. 1984 November; 81(21):6851-5), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody including one antigen-combining site having specificity for an antigen and another antigen-combining site having specificity for a different antigen.

(iii) Humanized Antibodies:

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones P T, et al., Nature. 1986 May 29-Jun. 4; 321(6069):522-5; Riechmann L, et al., Nature. 1988 Mar. 24; 332(6162):323-7; Verhoeyen M, et al., Science. 1988 Mar. 25; 239(4847):1534-6), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims M J, et al., J Immunol. 1993 Aug. 15; 151(4):2296-308; Chothia C & Lesk A M. J Mol Biol. 1987 Aug. 20; 196(4):901-17). Another method uses a particular framework region derived from the consensus sequence of all human antibodies of a particular subgroup of light or heavy chains. The same framework may be used for several different humanized antibodies (Carter P, et al., Proc Natl Acad Sci USA. 1992 May 15; 89(10):4285-9; Presta L G, et al., J Immunol. 1993 Sep. 1; 151(5):2623-32).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen, is achieved. In general, the hypervariable region residues are directly and most substantially involved in influencing antigen binding.

(iv) Human Antibodies:

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits A, et al., Proc Natl Acad Sci USA. 1993 Mar. 15; 90(6):2551-5; Nature. 1993 Mar. 18; 362(6417):255-8; Bruggemann M, et al., Year Immunol. 1993;7:33-40; and U.S. Pat. Nos. 5,591,669; 5,589,369 and 5,545,807. Alternatively, phage display technology (McCafferty J, et al., Nature. 1990 Dec. 6; 348(6301):552-4) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats ; for their review see, e.g., Johnson K S & Chiswell D J. Curr Opin Struct Biol. 1993; 3:564-71. Several sources of V-gene segments can be used for phage display.

Clackson T, et al., Nature. 1991 Aug. 15; 352(6336):624-8 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks J D, et al., J Mol Biol. 1991 Dec. 5; 222(3):581-97, or Griffiths A D, et al., EMBO J. 1993 February; 12(2):725-34. See, also, U.S. Pat. Nos. 5,565,332 and 5,573,905.

Human antibodies may also be generated by in vitro activated B cells (see U.S. Pat. Nos. 20 5,567,610 and 5,229,275). A preferred means of generating human antibodies using SCID mice is disclosed in commonly-owned, co-pending applications.

(v) Antibody Fragments:

Various techniques have been developed for the production of antibody fragments. Traditionally, these fragments were derived via proteolytic digestion of intact antibodies (see, e.g., Morimoto K & Inouye K. J Biochem Biophys Methods. 1992 March; 24(1-2):107-17; Brennan M, et al., Science. 1985 Jul. 5; 229(4708):81-3). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′) 2 fragments (Carter P, et al., Biotechnology (NY). 1992 February; 10(2):163-7). According to another approach, F (ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894 and 5,587,458. The antibody fragment may also be a “linear antibody”, e.g., as described in U.S. Pat. No.5,641,870 for example. Such linear antibody fragments may be monospecific or bispecific.

(vi) Non-Antibody Binding Protein:

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light variable regions of an antibody connected by a peptide linker and will fold into a structure similar to that of the two peptides antibody. The single-chain binding molecule displays several advantages over conventional antibodies, including, smaller size, greater stability and are more easily modified.

Ku et al. (Proc Natl Acad Sci USA 92(14):6552-6556 (1995)) describe an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) describe an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc Natl Acad Sci USA 96(5):1898-1903 (1999)) describe an antibody mimic based on a lipocalin scaffold (Anticalin(registered trademark)). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin(registered trademark) would be suitable to be used as an alternative to antibodies.

Anticalins (registered trademark) are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) describe a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric confirmation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol. 49(2):209-216 (2003)) describe a methodology for reducing antibodies into smaller peptidomimetics, they term “antibody like binding peptidomimetics” (ABiP) which can also be useful as an alternative to antibodies.

Silverman et al. (Nat Biotechnol. (2005), 23: 1556-1561) describe fusion proteins that are single-chain polypeptides including multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds including, but not limited to, RNA molecules and unnatural oligomers (e.g., protease inhibitors, benzodiazepines, purine derivatives and beta-turn mimics) all of which are suitable for use with the present invention.

(vii) Pharmaceutical Formulations:

Therapeutic formulations of present antibodies used in accordance with the present invention may be prepared for storage by mixing an anti-SYNGR4 antibody having the desired degree of purity with optional pharmaceutically acceptable carriers, excipients or stabilizers (Remington: The Science and Practice of Pharmacy, 21^{st} Ed., Lippincott, Williams and Wilkins, 2005), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, his tidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e. g. Zn-protein complexes); and/or non-ionic surfactants such as TWEEN™, PLURONTCS™ or polyethylene glycol (PEG). Lyophilized formulations adapted for subcutaneous administration are described in WO97/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a chemotherapeutic agent, cytokine or immunosuppressive agent. The effective amount of such other agents depends on the amount of antibody present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy, 21^{st} Ed., Lippincott, Williams and Wilkins, 2005.

Sustained-release preparations may be prepared. Suitable examples of sustained release preparations include semipermeable matrices of solid hydrophobic polymers containing the agent, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L-glutamate, noir degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D-(−)-3-hydroxybutyric acid. The formulations to be used for in vivo administration must be sterile. This is readily accomplished by filtration through sterile filtration membranes.

(x) Treatment with an Antibody:

A composition including anti-SYNGR4 antibodies may be formulated, dosed, and administered in a fashion consistent with good medical practice. Preferably, the present antibody will be a human, chimeric or humanized antibody scFv, or antibody fragment. Factors for consideration in this context include the particular lung cancer being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the antibody to be administered will be governed by such considerations.

As a general proposition, the therapeutically effective amount of the antibody administered parenterally per dose will be in the range of about 0.1 to 20 mg/kg of patient body weight per day, with the typical initial range of antibody used being in the range of about 2 to 10 mg/kg.

As noted above, however, these suggested amounts of antibody are subject to a great deal of therapeutic discretion. The key factor in selecting an appropriate dose and scheduling is the result obtained, as indicated above.

For example, relatively higher doses may be needed initially for the treatment of ongoing and acute diseases. To obtain the most efficacious results, depending on the disease or disorder, the antibody may be administered as close to the first sign, diagnosis, appearance, or occurrence of the disease or disorder as possible or during remissions of the disease or disorder.

The antibody may be administered by any suitable means, including parenteral, subcutaneous, intraperitoneal, intrapulmonary, inhalational and intranasal, and, if desired for local immunosuppressive treatment, intralesional administration. Parenteral infusions include intramuscular, intravenous, intraarterial, intraperitoneal, or subcutaneous administration.

In addition, the antibody may suitably be administered by pulse infusion, e.g., with declining doses of the antibody. Preferably the dosing is given by injections, most preferably intravenous or subcutaneous injections, depending in part on whether the administration is brief or chronic.

One additionally may administer other compounds, such as cytotoxic agents, chemotherapeutic agents, immunosuppressive agents and/or cytokines with the antibody herein. The combined administration includes co-administration, using separate formulations or a single pharmaceutical formulation, and consecutive administration in either order, wherein preferably there is a time period while both (or all) active agents simultaneously exert their biological activities.

Aside from administration of the antibody to the patient, the present invention contemplates administration of the antibody by gene therapy. Such administration of a nucleic acid encoding an antibody is encompassed by the expression “administering a therapeutically effective amount of an antibody”. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate pre-cipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262: 4429-4432 (1987); and Wagner et al, Proc. Nad. Acad. Sci. USA 87: 3410-3414 (1990). For review of the currently known gene marking and gene therapy protocols see Anderson et al., Science 256: 808-813 (1992). See also WO 93/25673 and the references cited therein.

Double Stranded Molecules

As used herein, the term “double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)). In some embodiments, the double-stranded molecules are isolated or recombinant.

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes an SYNGR4 sense nucleic acid sequence (also referred to as “sense strand”), an SYNGR4 antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions is sufficient such that base pairing occurs between the regions, the first and second regions are joined by a loop region, the loop results from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes a SYNGR4 sense nucleic acid sequence (also referred to as “sense strand”), a SYNGR4 antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may include not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of the first and second regions complementary to one another, i.e., sense and antisense strands. The degree of complementarity and orientation of the regions is sufficient such that base pairing occurs between the regions, the first and second regions are joined by a loop region, the loop results from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, examples of isolated nucleic acid includes DNA, RNA, and derivatives thereof.

A double-stranded molecule against SYNGR4, which molecule hybridizes to target mRNA, decreases or inhibits production of SYNGR4 protein encoded by SYNGR4 gene by associating with the normally single-stranded mRNA transcript of the gene, thereby interfering with translation and thus, inhibiting expression of the protein. As demonstrated herein, the expression of SYNGR4 in lung cancer cell lines was inhibited by dsRNA that specifically annealed to the SYNGR4 encoding gene (FIG. 3A). Therefore the present invention provides isolated double-stranded molecules that inhibit the expression of SYNGR4 gene when introduced into a cell expressing the SYNGR4 gene. The target sequence of double-stranded molecule may be designed by an siRNA design algorithm such as that mentioned below.

SYNGR4 target sequence includes, for example, nucleotides

SEQ ID NO: 11 (positions 389-407nt of SEQ ID NO: 13)

SEQ ID NO: 12 (positions 754-772nt of SEQ ID NO: 13)

Specifically, the present invention provides the following double-stranded molecules [1] to [18]:

[1] An isolated double-stranded molecule that, when introduced into a cell, specifically inhibits expression of SYNGR4, such molecule composed of a sense strand and an antisense strand complementary thereto, hybridized to each other to form the double-stranded molecule;

[2] The double-stranded molecule of [1], wherein said double-stranded molecule acts on SYNGR4 mRNA, matching a target sequence selected from among SEQ ID NO: 11 (at the position of 389-407nt of SEQ ID NO: 13), SEQ ID NO: 12 (at the position of 754-772nt of SEQ ID NO: 13);

[3] The double-stranded molecule of [2], wherein the sense strand contains a sequence corresponding to a target sequence selected from among SEQ ID NOs: 11, 12, 19 and 20;

[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6] having a length of less than about 25 nucleotides;

[8] The double-stranded molecule of [7], having a length of between about 19 and about 25 nucleotides;

[9] The double-stranded molecule of [1], composed of a single polynucleotide having both the sense and antisense strands linked by an intervening single-strand;

[10] The double-stranded molecule of [9], having the general formula 5′-[A]-[B]-[A′]-3′, wherein [A] is the sense strand containing a sequence corresponding to a target sequence selected from among SEQ ID NOs: 11, 12, 19 and 20, [B] is the intervening single-strand composed of 3 to 23 nucleotides, and [A′] is the antisense strand containing a sequence complementary to [A];

[11] The double-stranded molecule of [1], composed of RNA;

[12] The double-stranded molecule of [1], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [2], wherein the molecule contains 3′ overhang;

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (on the worldwide web at ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA dinucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as

SEQ ID NO: 11, 12, 19 and 20 for SYNGR4 gene,

Double-stranded molecules targeting the above-mentioned target sequences were respectively examined for their ability to suppress the growth of cells expressing the target genes. Therefore, the present invention provides double-stranded molecules targeting any of the sequences selected from the group of

SEQ ID NO: 11 (at the position 389-407nt of SEQ ID NO: 13), 12 (at the position 754-772nt of SEQ ID NO: 13), 19 (at the position 519-537nt of SEQ ID NO: 13) and 20 (at the position 520-538nt of SEQ ID NO: 13) for SYNGR4 gene,

The double-stranded molecule of the present invention may be directed to a single target SYNGR4 gene sequence or may be directed to a plurality of target SYNGR4 gene sequences.

A double-stranded molecule of the present invention targeting the above-mentioned targeting sequence of SYNGR4 gene include isolated polynucleotides that contain any of the nucleic acid sequences of target sequences and/or complementary sequences to the target sequences. Examples of polynucleotides targeting SYNGR4 gene include those containing the sequence of SEQ ID NO: 11, 12, 19 or 20 and/or complementary sequences to these nucleotides; However, the present invention is not limited to these examples, and minor modifications in the aforementioned nucleic acid sequences are acceptable so long as the modified molecule retains the ability to suppress the expression of SYNGR4 gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of SYNGR4 genes or antisense strands complementary thereto were tested in vitro for their ability to decrease production of SYNGR4 gene product in lung cancer cell lines (e.g., using A549 and SBC-5) according to standard methods. Furthermore, for example, reduction in SYNGR4 gene product in cells contacted with the candidate double-stranded molecule compared to cells cultured in the absence of the candidate molecule can be detected by, e.g. RT-PCR using primers for SYNGR4 mRNA mentioned under Example 1 item “Semi-quantitative RT-PCR”. Sequences which decrease the production of SYNGR4 gene product in in vitro cell-based assays can then be tested for there inhibitory effects on lung cancer cell growth. Sequences which inhibit lung cancer cell growth in in vitro cell-based assay can then be tested for their in vivo ability using animals with lung cancer, e.g. nude mouse xenograft models, to confirm decreased production of SYNGR4 product and decreased lung cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 1000 nucleotides in length for SYNGR4. For example, the polynucleotide is less than 500, 200, 100, 75, 50, or 25 nucleotides in length for all of the genes. The isolated polynucleotides of the present invention are useful for forming double-stranded molecules against SYNGR4 gene or preparing template DNAs encoding the double-stranded molecules. When the polynucleotides are used for forming double-stranded molecules, the sense strand of polynucleotide may be longer than 19 nucleotides, preferably longer than 21 nucleotides, and more preferably has a length of between about 19 and 25 nucleotides. Accordingly, the present invention provides the double-stranded molecules including a sense strand and an antisense strand, wherein the sense strand includes a nucleotide sequence corresponding to a target sequence. In preferable embodiments, the sense strand hybridizes with antisense strand at the target sequence to form the double-stranded molecule having between 19 and 25 nucleotide pair in length.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deaza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may include both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

sense strand:
5′-[---DNA---]-3′
3′-(RNA)-[DNA]-5′:
antisense strand,
sense strand:
5′-(RNA)-[DNA]-3′
3′-(RNA)-[DNA]-5′:
antisense strand,
and
sense strand:
5′-(RNA)-[DNA]-3′
3′-(---RNA---)-5′:
antisense strand.

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA includes the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotides of SEQ ID NOs: 11, 12, 19 and 20 for SYNGR4.

The present invention is not limited to these examples, and the target sequence in [A] may be modified sequences from these examples so long as the double-stranded molecule retains the ability to suppress the expression of the targeted SYNGR4 gene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (on the worldwide web at ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 Jun. 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence SEQ ID NO: 11)
CAAGAUGGAGUCUCCGCAG-[B]-CUGCGGAGACUCCAUCUUG;
(for target sequence SEQ ID NO: 12)
AUGAUGCUCCAGUCCCUUA-[B]-UAAGGGACUGGAGCAUCAU;
(for target sequence SEQ ID NO: 19)
CGCAUUGCCGGCACCCGCU-[B]-AGCGGGTGCCGGCAATGCG;
(for target sequence SEQ ID NO: 20)
GCAUUGCCGGCACCCGCUU-[B]-AAGCGGGTGCCGGCAATGC;

Furthermore, in order to enhance the inhibition activity of the double-stranded molecules, nucleotide “u” can be added to 3′end of the antisense strand of the target sequence, as 3′ overhangs. The number of “u”s to be added is at least 2, generally 2 to 10, preferably 2 to 5. The added “u”s form single strand at the 3′end of the antisense strand of the double-stranded molecule.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking SYNGR4 sequences may be identical or different, such that their expression can be modulated independently, or in a temporal or spatial manner. The double-stranded molecules can be transcribed intracellularly by cloning SYNGR4 gene templates into a vector containing, e.g., a RNA pol III transcription unit from the small nuclear RNA (snRNA) U6 or the human H1 RNA promoter.

Vectors Containing a Double-Stranded Molecule of the Present Invention:

Also included in the present invention are vectors containing one or more of the double-stranded molecules described herein, and a cell containing such a vector.

Specifically, the present invention provides the following vector of [1] to [10].

[1] A vector, encoding a double-stranded molecule that, when introduced into a cell, specifically inhibits expression of SYNGR4, such molecule composed of a sense strand and an antisense strand complementary thereto, hybridized to each other to form the double-stranded molecule.

[2] The vector of [1], encoding the double-stranded molecule acts on mRNA, matching a target sequence selected from among SEQ ID NO: 11 (at the position of 389-407nt of SEQ ID NO: 13), SEQ ID NO: 12 (at the position of 754-772nt of SEQ ID NO: 13), SEQ ID NO:19 (at the position 519-537nt of SEQ ID NO: 13) and SEQ ID NO:20 (at the position 520-538nt of SEQ ID NO: 13);

[3] The vector of 1], wherein the sense strand contains a sequence corresponding to a target sequence selected from among SEQ ID NOs: 11, 12, 19 and 20;

[4] The vector of [3], encoding the double-stranded molecule, wherein the sense strand of the double-stranded molecule hybridizes with antisense strand at the target sequence to form the double-stranded molecule having a length of less than about 100 nucleotides;

[5] The vector of [4], encoding the double-stranded molecule, wherein the sense strand of the double-stranded molecule hybridizes with antisense strand at the target sequence to form the double-stranded molecule having a length of less than about 75 nucleotides;

[6] The vector of [5], encoding the double-stranded molecule, wherein the sense strand of the double-stranded molecule hybridizes with antisense strand at the target sequence to form the double-stranded molecule having a length of less than about 50 nucleotides;

[7] The vector of [6] encoding the double-stranded molecule, wherein the sense strand of the double-stranded molecule hybridizes with antisense strand at the target sequence to form the double-stranded molecule having a length of less than about 25 nucleotides;

[8] The vector of [7], encoding the double-stranded molecule, wherein the sense strand of the double-stranded molecule hybridizes with antisense strand at the target sequence to form the double-stranded molecule having a length of between about 19 and about 25 nucleotides;

[9] The vector of [1], wherein the double-stranded molecule is composed of a single polynucleotide having both the sense and antisense strands linked by an intervening single-strand;

[10] The vector of [9], encoding the double-stranded molecule having the general formula 5′[A]-[B]-[A′]-3′, wherein [A] is the sense strand containing a sequence corresponding to a target sequence selected from among SEQ ID NOs: 11, 12, 19 and 20, [B] is the intervening single-strand composed of 3 to 23 nucleotides, and [A′] is the antisense strand containing a sequence complementary to [A];

A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Alternatively, the present invention provides vectors including each of a combination of polynucleotide including a sense strand nucleic acid and an antisense strand nucleic acid, wherein said sense strand nucleic acid includes nucleotide sequence of SEQ ID NOs: 11, 12, 19 and 20, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the SYNGR4 gene, inhibits expression of said gene. Preferably, the polynucleotide is an oligonucleotide of between about 19 and 25 nucleotides in length (e.g., contiguous nucleotides from the nucleotide sequence of SEQ ID NO: 13). More preferably, the combination of polynucleotide includes a single nucleotide transcript including the sense strand and the antisense strand linked via a single-stranded nucleotide sequence. More preferably, the combination of polynucleotide has the general formula 5′[A]-[B]-[A′]-3′, wherein [A] is a nucleotide sequence including SEQ ID NO: 11, 12, 19 and 20; [B] is a nucleotide sequence consisting of about 3 to about 23 nucleotide; and [A′] is a nucleotide sequence complementary to [A].

Vectors of the present invention can be produced, for example, by cloning SYNGR4 sequence into an expression vector so that regulatory sequences are operatively-linked to SYNGR4 sequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

Methods of Inhibiting or Reducing Growth of a Cancer Cell and Treating Cancer Using a Double-Stranded Molecule of the Present Invention:

The ability of certain siRNA to inhibit NSCLC has been previously described in WO 2005/89735, incorporated by reference herein. In the present invention, two different dsRNA for SYNGR4 were tested for their ability to inhibit lung cancer cell growth. The two dsRNA for SYNGR4 (FIGS. 3A,3B), effectively knocked down the expression of the gene in lung cancer cell lines coincided with suppression of cell proliferation.

Therefore, the present invention provides methods for inhibiting lung cancer cell growth, by inhibiting the expression of SYNGR4. SYNGR4 gene expression can be inhibited by any method known in the art, including use of the aforementioned double-stranded molecules which specifically target of SYNGR4 gene or the aforementioned vectors that express double-stranded molecules which specifically target the SYNGR4 gene.

Such ability of the present double-stranded molecules and vectors to inhibit cell growth of lung cancer cells indicates that they can be used for methods for treating and/or preventing lung cancer. Thus, the present invention provides methods to treat patients with lung cancer by administering a double-stranded molecule against SYNGR4 gene or a vector expressing the molecule without adverse side effects because the SYNGR4 gene is not overexpressed in normal tissues (FIGS. 1A, 2A and B).

Specifically, the present invention provides the following methods [1] to [36]:

[1] A method for inhibiting a growth of cancer cell and/or treating a cancer, wherein the cancer cell or the cancer over-expresses the SYNGR4 gene, which method includes the step of contacting the cell with at least one isolated double-stranded molecule that specifically inhibits the expression of SYNGR4 in a cancer cell over-expressing the gene, thereby inhibiting the growth of the lung cancer cell and/or treating the lung cancer.

[2] The method of [1], wherein the double-stranded molecule acts at SYNGR4 mRNA which matches a target sequence selected from among SEQ ID NO: 11 (at the position of 389-407nt of SEQ ID NO: 13) and SEQ ID NO: 12 (at the position of 754-772nt of SEQ ID NO: 13), SEQ ID NO:19 (at the position 519-537nt of SEQ ID NO: 13) and SEQ ID NO:20 (at the position 520-538nt of SEQ ID NO: 13).

[3] The method of [2], wherein the sense strand contains the sequence corresponding to a target sequence selected from among SEQ ID NOs: 11, 12, 19 and 20.

[4] The method of [1], wherein the cancer to be treated is lung cancer;

[5] The method of [4], wherein the lung cancer is NSCLC or SCLC;

[6] The method of [1], wherein a plurality of double-stranded molecules that specifically inhibit the expression of SYNGR4 are administered;

[7] The method of [3], wherein the sense strand of the double-stranded molecule has a length of less than about 100 nucleotides;

[8] The method of [7], wherein the sense strand of the double-stranded molecule has a length of less than about 75 nucleotides;

[9] The method of [8], wherein the sense strand of the double-stranded molecule has a length of less than about 50 nucleotides;

[10] The method of [9], wherein the sense strand of the double-stranded molecule has a length of less than about 25 nucleotides;

[11] The method of [10], wherein the sense strand of the double-stranded molecule has a length of between about 19 and about 25 nucleotides in length;

[12] The method of [1], wherein the double-stranded molecule is composed of a single polynucleotide containing both the sense strand and the antisense strand linked by an intervening single-strand;

[13] The method of [12], wherein the double-stranded molecule has the general formula 5′-[A]-[B]-[A′]-3′, wherein [A] is the sense strand containing a sequence corresponding to a target sequence selected from among SEQ ID NOs: 11, 12, 19 and 20, [B] is the intervening single strand composed of 3 to 23 nucleotides, and [A′] is the antisense strand containing a sequence complementary to [A];

[14] The method of [1], wherein the double-stranded molecule is an RNA;

[15] The method of [1], wherein the double-stranded molecule contains both DNA and RNA;

[16] The method of [15], wherein the double-stranded molecule is a hybrid of a DNA polynucleotide and an RNA polynucleotide;

[17] The method of [16] wherein the sense and antisense strand polynucleotides are composed of DNA and RNA, respectively;

[18] The method of [15], wherein the double-stranded molecule is a chimera of DNA and RNA;

[19] The method of [18], wherein a region flanking to the 3′-end of the antisense strand, or both of a region flanking to the 5′-end of sense strand and a region flanking to the 3′-end of antisense strand are composed of RNA;

[20] The method of [19], wherein the flanking region is composed of 9 to 13 nucleotides;

[21] The method of [1], wherein the double-stranded molecule contains 3′ overhangs;

[22] The method of [1], wherein the double-stranded molecule is contained in a composition which includes, in addition to the molecule, a transfection-enhancing agent and pharmaceutically acceptable carrier.

[23] The method of [1], wherein the double-stranded molecule is encoded by a vector;

[24] The method of [23], wherein the double-stranded molecule encoded by the vector acts at mRNA which matches a target sequence selected from among SEQ ID NO: 11 (at the position of 389-407nt of SEQ ID NO: 13), SEQ ID NO: 12 (at the position of 754-772nt of SEQ ID NO: 13), SEQ ID NO:19 (at the position 519-537nt of SEQ ID NO: 13) and SEQ ID NO:20 (at the position 520-538nt of SEQ ID NO: 13).

[25] The method of [24], wherein the sense strand of the double-stranded molecule encoded by the vector contains the sequence corresponding to a target sequence selected from among SEQ ID NOs: 11, 12, 19 and 20.

[26] The method of [23], wherein the cancer to be treated is lung cancer;

[27] The method of [26], wherein the lung cancer is NSCLC or SCLC;

[28] The method of [23], wherein plural kinds of the double-stranded molecules are administered;

[29] The method of [25], wherein the sense strand of the double-stranded molecule encoded by the vector has a length of less than about 100 nucleotides;

[30] The method of [29], wherein the sense strand of the double-stranded molecule encoded by the vector has a length of less than about 75 nucleotides;

[31] The method of [30], wherein the sense strand of the double-stranded molecule encoded by the vector has a length of less than about 50 nucleotides;

[32] The method of [31], wherein the sense strand of the double-stranded molecule encoded by the vector has a length of less than about 25 nucleotides;

[33] The method of [32], wherein the sense strand of the double-stranded molecule encoded by the vector has a length of between about 19 and about 25 nucleotides in length;

[34] The method of [23], wherein the double-stranded molecule encoded by the vector is composed of a single polynucleotide containing both the sense strand and the antisense strand linked by an intervening single-strand;

[35] The method of [34], wherein the double-stranded molecule encoded by the vector has the general formula 5′-[A]-[B]-A′]-3′, wherein [A] is the sense strand containing a sequence corresponding to a target sequence selected from among SEQ ID NOs: 11, 12, 19 and 20, [B] is a intervening single-strand is composed of 3 to 23 nucleotides, and [A′] is the antisense strand containing a sequence complementary to [A]; and

[36] The method of [23], wherein the double-stranded molecule encoded by the vector is contained in a composition which includes, in addition to the molecule, a transfection-enhancing agent and pharmaceutically acceptable carrier. The methods of the present invention will be described in more detail below.

The growth of cells expressing SYNGR4 gene may be inhibited by contacting the cells with a double-stranded molecule that specifically anneals to the SYNGR4 gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, including, e.g., using the MTT cell proliferation assay.

The growth of any kind of cell may be suppressed according to the present method so long as the cell expresses or over-expresses SYNGR4, the target gene of the double-stranded molecule of the present invention. Exemplary cells include lung cancer cells, including both NSCLC and SCLC.

Thus, patients suffering from or at risk of developing disease caused or promoted in part by the overexpression of SYNGR4 may be treated by administering at least one of the present double-stranded molecules, at least one vector expressing at least one of the molecules or at least one composition containing at least one of the molecules. For example, patients of lung cancer may be treated according to the present methods. The type of cancer may be identified by standard methods according to the particular type of tumor to be diagnosed. Lung cancer may be diagnosed, for example, with Carcinoembryonic antigen (CEA), CYFRA, pro-GRP and so on, as lung cancer marker, or with Chest X-Ray and/or Sputum Cytology. More preferably, patients treated by the methods of the present invention are selected by detecting the expression of SYNGR4 in a biopsy from the patient by RT-PCR or immunoassay. Preferably, before the treatment of the present invention, the biopsy specimen from the subject is confirmed for SYNGR4 gene over-expression by methods known in the art, for example, immunohistochemical analysis or RT-PCR.

According to the present method to inhibit lung cancer cell growth and thereby treating lung cancer, when administering a plurality of double-stranded molecules (or vectors expressing or compositions containing the same), each of the molecules may have different structures but act at mRNA which matches the same target sequence of SYNGR4. Alternatively, a plurality of double-stranded molecules may act at mRNA which matches different target sequences within the SYNGR4 gene or acts at mRNA which matches different target sequence of different gene. For example, the method may utilize double-stranded molecules directed to SYNGR4. Alternatively, for example, the method may utilize double-stranded molecules directed to one, two or more target sequence of within the SYNGR4 coding sequence.

For inhibiting lung cancer cell growth, a double-stranded molecule of the present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

The term “specifically inhibit” in the context of inhibitory polynucleotides and polypeptides refers to the ability of an agent or ligand to preferentially inhibit the expression or the biological function of SYNGR4 in comparison to the expression or biological function of polynucleotides and polypeptides other than SYNGR4. Specific inhibition typically results in at least about a 2-fold inhibition over background, preferably greater than about 10 fold and most preferably greater than 100-fold inhibition of 10 fold expression (e.g., transcription or translation) or measured biological function (e.g., cell growth or proliferation, inhibition of apoptosis, intracellular signaling from SYNGR4). Expression levels and/or biological function can be measured in the context of comparing treated and untreated cells, or a cell population before and after treatment. In some embodiments, the expression or biological function of SYNGR4 is completely inhibited. Typically, specific inhibition is a statistically meaningful reduction in SYNGR4 expression or biological function (e.g., p<=0.05) using an appropriate statistical test.

A treatment is deemed “efficacious” if it leads to clinical benefit such as, reduction in expression of SYNGR4 gene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecules of the invention degrade the SYNGR4 mRNA in substoichiometric amounts. Without wishing to be bound by any theory, it is believed that the double-stranded molecules of the invention cause degradation of the target mRNA in a catalytic manner. Thus, compared to standard cancer therapies, significantly less double-stranded molecule needs to be delivered at or near the site of cancer to exert a therapeutic effect.

One skilled in the art can readily determine an effective amount of the double-stranded molecules of the invention to be administered to a given subject, by taking into account factors such as body weight, age, sex, type of disease, symptoms and other conditions of the subject; the route of administration; and whether the administration is regional or systemic. Generally, an effective amount of the double-stranded molecules of the invention is an intracellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of a cancer expressing SYNGR4; for example lung cancer, especially NSCLC or SCLC. In particular, a double-stranded molecule containing a target sequence of SYNGR4 (i.e., SEQ ID NO: 11, 12, 19 or 20) is particularly preferred for the treatment of lung cancer. For treating cancer, the double-stranded molecules of the invention can also be administered to a subject in combination with a pharmaceutical agent different from the double-stranded molecule. Alternatively, the double-stranded molecules of the invention can be administered to a subject in combination with another therapeutic method designed to treat cancer. For example, the double-stranded molecules of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as lung tumor tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule includes a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

In particular, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can include both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Mirus Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecules of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, inhalational or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection); subcutaneous injection or deposition including subcutaneous infusion (such as by osmotic pumps); direct application to the area at or near the site of cancer, for example by a catheter or other placement device (e.g., a suppository or an implant including a porous, non-porous, or gelatinous material); and inhalation. It is preferred that injections or infusions of the double-stranded molecule or vector be given at or near the site of cancer.

The double-stranded molecules of the invention can be administered in a single dose or in multiple doses. Where the administration of the double-stranded molecules of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecules of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen includes multiple administrations, it is understood that the effective amount of a double-stranded molecule administered to the subject can include the total amount of a double-stranded molecule administered over the entire dosage regimen.

Compositions Containing a Double-Stranded Molecule of the Present Invention:

In addition to the above, the present invention also provides pharmaceutical compositions that include at least one of the present double-stranded molecules or the vectors coding for the molecules. Specifically, the present invention provides the following compositions [1] to [36]:

[1] A composition for inhibiting a growth of cancer cell and treating a cancer, wherein the cancer cell and the cancer over-expresses the SYNGR4 gene, including at least one isolated double-stranded molecule inhibiting the expression of SYNGR4 and the cell proliferation, which molecule is composed of a sense strand and an antisense strand complementary thereto, hybridized to each other to form the double-stranded molecule.

[2] The composition of [1], wherein the double-stranded molecule acts at mRNA which matches a target sequence selected from among SEQ ID NO: 11 (at the position of 389-407nt of SEQ ID NO: 13), SEQ ID NO:12 (at the position of 754-772nt of SEQ ID NO: 13), SEQ ID NO:19 (at the position 519-537nt of SEQ ID NO: 13) and SEQ ID NO:20 (at the position 520-538nt of SEQ ID NO: 13).

[3] The composition of [2], wherein the double-stranded molecule, wherein the sense strand contains a sequence corresponding to a target sequence selected from among SEQ ID NOs: 11, 12, 19 and 20.

[4] The composition of [1], wherein the cancer to be treated is lung cancer;

[5] The composition of [4], wherein the lung cancer is NSCLC or SCLC;

[6] The composition of [1], wherein the composition contains plural kinds of the double-stranded molecules;

[7] The composition of [3], wherein the sense strand of the double-stranded molecule has a length of less than about 100 nucleotides;

[8] The composition of [7], wherein the sense strand of the double-stranded molecule has a length of less than about 75 nucleotides;

[9] The composition of [8], wherein the sense strand of the double-stranded molecule has a length of less than about 50 nucleotides;

[10] The composition of [9], wherein the sense strand of the double-stranded molecule has a length of less than about 25 nucleotides;

[11] The composition of [10], wherein the sense strand of the double-stranded molecule has a length of between about 19 and about 25 nucleotides;

[12] The composition of [1], wherein the double-stranded molecule is composed of a single polynucleotide containing the sense strand and the antisense strand linked by an intervening single-strand;

[13] The composition of [12], wherein the double-stranded molecule has the general formula 5′-[A]-[B]-[A′]-3′, wherein [A] is the sense strand sequence contains a sequence corresponding to a target sequence selected from among SEQ ID NOs: 11, 12, 19 and 20, [B] is the intervening single-strand consisting of 3 to 23 nucleotides, and [A′] is the antisense strand contains a sequence complementary to [A];

[14] The composition of [1], wherein the double-stranded molecule is an RNA;

[15] The composition of [1], wherein the double-stranded molecule is DNA and/or RNA;

[16] The composition of [15], wherein the double-stranded molecule is a hybrid of a DNA polynucleotide and an RNA polynucleotide;

[17] The composition of [16], wherein the sense and antisense strand polynucleotides are composed of DNA and RNA, respectively;

[18] The composition of [15], wherein the double-stranded molecule is a chimera of DNA and RNA;

[19] The composition of [18], wherein a region flanking to the 3′-end of the antisense strand, or both of a region flanking to the 5′-end of sense strand and a region flanking to the 3′-end of antisense strand are composed of RNA;

[20] The composition of [19], wherein the flanking region is composed of 9 to 13 nucleotides;

[21] The composition of [1], wherein the double-stranded molecule contains 3′ overhangs;

[22] The composition of [1], wherein the composition includes a transfection-enhancing agent and pharmaceutically acceptable carrier.

[23] The composition of [1], wherein the double-stranded molecule is encoded by a vector and contained in the composition;

[24] The composition of [23], wherein the double-stranded molecule encoded by the vector acts at mRNA which matches a target sequence selected from among SEQ ID NO: 11 (at the position of 389-407nt of SEQ ID NO: 13), SEQ ID NO: 12 (at the position of 754-772nt of SEQ ID NO: 13), SEQ ID NO:19 (at the position 519-537nt of SEQ ID NO: 13) and SEQ ID NO:20 (at the position 520-538nt of SEQ ID NO: 13).

[25] The composition of [24], wherein the sense strand of the double-stranded molecule encoded by the vector contains the sequence corresponding to a target sequence selected from among SEQ ID NOs: 11, 12, 19 and 20.

[26] The composition of [23], wherein the cancer to be treated is lung cancer;

[27] The composition of [26], wherein the lung cancer is NSCLC or SCLC;

[28] The composition of [23], wherein plural kinds of the double-stranded molecules are administered;

[29] The composition of [25], wherein the sense strand of the double-stranded molecule encoded by the vector has a length of less than about 100 nucleotides;

[30] The composition of [29], wherein the sense strand of the double-stranded molecule encoded by the vector has a length of less than about 75 nucleotides;

[31] The composition of [30], wherein the sense strand of the double-stranded molecule encoded by the vector has a length of less than about 50 nucleotides;

[32] The composition of [31], wherein the sense strand of the double-stranded molecule encoded by the vector has a length of less than about 25 nucleotides;

[33] The composition of [32], wherein the sense strand of the double-stranded molecule encoded by the vector has a length of between about 19 and about 25 nucleotides in length;

[34] The composition of [23], wherein the double-stranded molecule encoded by the vector is composed of a single polynucleotide containing both the sense strand and the antisense strand linked by an intervening single-strand;

[35] The composition of [23], wherein the double-stranded molecule has the general formula 5′-[A]-[B]-[A′]-3′, wherein [A] is the sense strand containing a sequence corresponding to a target sequence selected from among SEQ ID NOs: 11, 12, 19 and 20, [B] is a intervening single-strand composed of 3 to 23 nucleotides, and [A′] is the antisense strand containing a sequence complementary to [A]; and

[36] The composition of [23], wherein the composition includes a transfection-enhancing agent and pharmaceutically acceptable carrier.

Suitable compositions of the present invention are described in additional detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington: The Science and Practice of Pharmacy, 21st ed., Lippincott, Williams and Wilkins. (2005), the entire disclosure of which is herein incorporated by reference.

The present pharmaceutical formulations contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain a plurality of double-stranded molecules, each of the molecules may be directed to the same target sequence, or different target sequences of SYNGR4. For example, the composition may contain double-stranded molecules directed to SYNGR4. Alternatively, for example, the composition may contain double-stranded molecules directed to one, two or more target sequences SYNGR4.

Furthermore, the present composition may contain a vector coding for one or a plurality of double-stranded molecules. For example, the vector may encode one, two or several kinds of the present double-stranded molecules. Alternatively, the present composition may contain a plurality of vectors, each of the vectors coding for a different double-stranded molecule.

Moreover, the present double-stranded molecules may be contained as liposomes in the present composition. See under the item of “Methods of treating cancer using the double-stranded molecule” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized. For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can include any of the carriers and excipients listed above and 10-95%, preferably 25-75%, of one or more double-stranded molecules of the invention. A pharmaceutical composition for aerosol (inhalational) administration can include 0.01-20% by weight, preferably 1-10% by weight, of one or more double-stranded molecules of the invention encapsulated in a liposome as described above, and propellant. A carrier can also be included as desired; e.g., lecithin for intranasal delivery.

In addition to the above, the present composition may contain other pharmaceutically active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a lung cancer characterized by the over-expression of SYNGR4. For example, the present invention relates to a use of double-stranded nucleic acid molecule inhibiting the expression of SYNGR4 gene in a cell, which molecule includes a sense strand and an antisense strand complementary thereto, hybridized to each other to form the double-stranded nucleic acid molecule and targets to a sequence selected from among SEQ ID NOs: 11, 12, 19 and 20, for manufacturing a pharmaceutical composition for treating lung cancer over-expressing SYNGR4.

Alternatively, the present invention further provides a method or process for manufacturing a pharmaceutical composition for treating a cancer caused or promoted in part by the overexpression of SYNGR4, e.g., a lung cancer characterized by the over-expression of SYNGR4, wherein the method or process includes a step for formulating a pharmaceutically or physiologically acceptable carrier with a double-stranded nucleic acid molecule inhibiting the expression of SYNGR4 in a cell, which over-expresses the gene, which molecule includes a sense strand and an antisense strand complementary thereto, hybridized to each other to form the double-stranded nucleic acid molecule and targets to a sequence selected from among SEQ ID NOs: 11, 12, 19 and 20 as active ingredients.

In another embodiment, the present invention also provides a method or process for manufacturing a pharmaceutical composition for treating a cancer caused or promoted in part by the overexpression of SYNGR4, e.g., a lung cancer characterized by the expression of SYNGR4, wherein the method or process includes a step for admixing an active ingredient with a pharmaceutically or physiologically acceptable carrier, wherein the active ingredient is a double-stranded nucleic acid molecule inhibiting the expression of SYNGR4 in a cell, which over-expresses the gene, which molecule includes a sense strand and an antisense strand complementary thereto, hybridized to each other to form the double-stranded nucleic acid molecule and targets to a sequence selected from among SEQ ID NOs: 11, 12, 19 and 20.

Method of Detecting or Diagnosing Lung Cancer

The expression of SYNGR4 was found to be specifically elevated in lung cancer cells (FIG. 1). Therefore, the genes identified herein as well as their transcription and translation products find diagnostic utility as markers for lung cancer and by measuring the expression of SYNGR4 in a lung tissue sample, lung cancer can be diagnosed. Specifically, the present invention provides a method for diagnosing lung cancer by determining the expression level of SYNGR4 in the subject. Lung cancers that can be diagnosed by the present method include NSCLC and SCLC. Furthermore, NSCLC, including lung adenocarcinoma and lung squamous cell carcinoma (SCC), can also be diagnosed or detected by the present invention.

According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

Alternatively, the present invention provides a method for detecting or identifying cancer cells in a subject-derived lung tissue sample, said method including the step of determining the expression level of the SYNGR4 gene in a subject-derived biological sample, wherein an increase in said expression level as compared to a normal control level of said gene indicates the presence or suspicion of cancer cells in the lung tissue.

Such results may be combined with additional information to assist a doctor, nurse, or other healthcare practitioner in diagnosing a subject as afflicted with the disease. In other words, the present invention may provide a doctor with useful information to diagnose a subject as afflicted with the disease. For example, according to the present invention, when there is doubt regarding the presence of cancer cells in the tissue obtained from a subject, clinical decisions can be reached by considering the expression level of the SYNGR4 gene, plus a different aspect of the disease including tissue pathology, levels of known tumor marker(s) in blood, and clinical course of the subject, etc. For example, some well-known diagnostic lung tumor markers in blood are IAP, ACT, BFP, CA19-9, CA50, CA72-4, CA130, CEA, KMO-1, NSE, SCC, SP1, Span-1, TPA, CSLEX, SLX, STN and CYFRA. Namely, in this particular embodiment of the present invention, the outcome of the gene expression analysis serves as an intermediate result for further diagnosis of a subject's disease state.

In another embodiment, the present invention provides a method for detecting a diagnostic marker of cancer, said method including the step of detecting the expression of the SYNGR4 gene in a subject-derived biological sample as a diagnostic marker of lung cancer. Specifically, the present invention provides the following methods [1] to [10]:

[1] A method for diagnosing lung cancer, said method including the steps of:

(a) detecting the expression level of the gene encoding the amino acid sequence of SYNGR4 in a biological sample; and

(b) correlating an increase in the expression level detected as compared to a normal control level of the gene to the presence of disease.

[2] The method of [1], wherein the expression level is at least 10% greater than the normal control level.

[3] The method of [1], wherein the expression level is detected by a method selected from among:

(a) detecting an mRNA including the sequence of SYNGR4,

(b) detecting a protein including the amino acid sequence of SYNGR4, and

(c) detecting a biological activity of a protein including the amino acid sequence of SYNGR4.

[4] The method of [1], wherein the lung cancer is NSCLC or SCLC.

[5] The method of [3], wherein the expression level is determined by detecting hybridization of a probe to a gene transcript of the gene.

[6] The method of [3], wherein the expression level is determined by detecting the binding of an antibody against the protein encoded by a gene as the expression level of the gene.

[7] The method of [1], wherein the biological sample includes biopsy, sputum or blood.

[8] The method of [1], wherein the subject-derived biological sample includes an epithelial cell.

[9] The method of [1], wherein the subject-derived biological sample includes a cancer cell.

[10] The method of [1], wherein the subject-derived biological sample includes a cancerous epithelial cell.

The method of diagnosing lung cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of SYNGR4. The biological samples include, but are not limited to, bodily tissues which are desired for diagnosing or are suspicion of suffering from cancer, and fluids, such as biopsy, blood, serum, plasma, saliva, sputum, pleural effusion and urine. Preferably, the biological sample contains a cell population including an epithelial cell, more preferably a cancerous epithelial cell or an epithelial cell derived from tissue suspected to be cancerous, e.g., lung tissue. Further, if necessary, the cell may be purified from the obtained bodily tissues and fluids, and then used as the biological sample.

According to the present invention, the expression level of SYNGR4 in the subject-derived biological sample is determined. The expression level can be determined at the transcription (nucleic acid) product level, using methods known in the art. For example, the mRNA of SYNGR4 may be quantified using probes by hybridization methods (e.g., Northern hybridization). The detection may be carried out on a chip or an array. The use of an array is preferable for detecting the expression level of a plurality of genes (e.g., various cancer specific genes) including SYNGR4. Those skilled in the art can prepare such probes utilizing the sequence information of the SYNGR4 (SEQ ID NO 13; GenBank accession number: NM_{—}012451). For example, the cDNA of SYNGR4 may be used as the probes. If necessary, the probe may be labeled with a suitable label, such as dyes, fluorescent and isotopes, and the expression level of the gene may be detected as the intensity of the hybridized labels. Furthermore, the transcription product of SYNGR4 may be quantified using primers by amplification-based detection methods (e.g., RT-PCR). Such primers can also be prepared based on the available sequence information of the gene. For example, the primers (SEQ ID NOs: 7 and 8) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of SYNGR4. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degrees Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees Centigrade for short probes or primers (e.g., 10 to 50 nucleotides) and at least about 60 degrees Centigrade for longer probes or primers. Stringent conditions may also be achieved with the addition of destabilizing agents, such as formamide.

Alternatively, the translation product may be detected for the diagnosis of the present invention. For example, the quantity of SYNGR4 protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to SYNGR4 protein. Methods to prepare these kinds of antibodies for the detection of proteins are well known in the art, and any method may be employed in the present invention to prepare such antibodies and equivalents thereof.

As another method to detect the expression level of SYNGR4 gene based on its translation product, the intensity of staining may be observed via immunohistochemical analysis using an antibody against SYNGR4 protein. Namely, the observation of strong staining indicates increased presence of the protein and at the same time high expression level of SYNGR4 gene.

Moreover, in addition to the expression level of SYNGR4 gene, the expression level of other cancer-associated genes, for example, genes known to be differentially expressed in lung cancer may also be determined to improve the accuracy of the diagnosis. The expression level of the SYNGR4 could also be correlated with a pathological determination of the cell and/or tissue for cancerous or pre-cancerous state.

The expression level of cancer marker genes, including the SYNGR4 gene, in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of SYNGR4 gene in samples from subjects whose disease state are known. Furthermore, the control level can be a database of expression patterns from previously tested cells. Moreover, according to an aspect of the present invention, the expression level of SYNGR4 gene in a biological sample may be compared to multiple control levels, which control levels are determined from multiple reference samples. It is preferred to use a control level determined from a reference sample derived from a tissue type similar to that of the patient-derived biological sample, e.g., lung tissue. Moreover, it is preferred, to use the standard value of the expression levels of SYNGR4 gene in a population with a known disease state. The standard value may be obtained by any method known in the art. For example, a range of mean +/−2 S.D. or mean +/−3 S.D. may be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of SYNGR4 gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference which is cancerous indicates that the subject is suffering from or at a risk of developing cancer.

Differences between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

Methods for Assessing the Prognosis of Cancer

The present invention relates, in part, to the discovery that SYNGR4 expression is significantly associated with poorer prognosis of patients with lung cancer. Thus, the present invention provides a method for determining or assessing the prognosis of a patient with a cancer caused or promoted in part by the over-expression of SYNGR4, in particular lung cancer, by detecting the expression level of the SYNGR4 in a biological sample of the patient; comparing the detected expression level to a control level; and determining a increased expression level of SYNGR4 in comparison to the normal control level as indicative of poor prognosis (poor survival). In other embodiments, determining a similar or increased expression level of SYNGR4 in comparison to a cancerous control level is indicative of a poor prognosis. Herein, the term “prognosis” refers to a forecast as to the probable outcome of the disease as well as the prospect of recovery from the disease as indicated by the nature and symptoms of the case. Accordingly, a less favorable, negative, or poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of SYNGR4 over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the SYNGR4 gene can be detected in the sample. The subject-derived biological sample may be any sample derived from a subject, e.g., a patient known to have or suspected of having lung cancer. Preferably, the biological sample is a lung cell (a cell obtained from the lung). Furthermore, the biological sample may include bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the SYNGR4 gene measured in the patient-derived biological sample, the poorer the prognosis for post-treatment remission, recovery, and/or survival and the higher the likelihood of poor clinical outcome. Thus, according to the present methods, the “control level” used for comparison may be, for example, the expression level of the SYNGR4 gene detected before any kind of treatment in an individual, or a population of individuals who showed good or positive prognosis of cancer after the treatment, which herein will be referred to as “good prognosis control level”. Alternatively, the “control level” may be the expression level of the SYNGR4 gene detected before any kind of treatment in an individual, or a population of individuals who showed poor or negative prognosis of cancer after the treatment, which herein will be referred to as “poor prognosis control level”. The “control level” is a single expression pattern derived from a single reference population or from a plurality of expression patterns. Thus, the control level may be determined based on the expression level of the SYNGR4 gene detected before any kind of treatment in a patient of cancer, or a population of the patients whose disease state (good or poor prognosis) is known. Preferably, cancer is lung cancer. It is preferred, to use the standard value of the expression levels of the SYNGR4 gene in a patient group with a known disease state. The standard value may be obtained by any method known in the art. For example, a range of mean +/−2 S.D. or mean +/−3 S.D. may be used as standard value.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the SYNGR4 gene in samples previously collected and stored from a control group. Furthermore, the control level can be a database of expression patterns from previously tested cells.

Moreover, according to an aspect of the present invention, the expression level of the SYNGR4 gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the SYNGR4 gene to a good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the SYNGR4 to the poor prognosis control level indicates a more favorable prognosis of the patient and a similarity in the expression level to the poor prognosis control level indicates less favorable, poorer prognosis for post-treatment remission, recovery, survival, and/or clinical outcome.

The expression level of the SYNGR4 gene in a biological sample can be considered altered when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the SYNGR4 genes.

The expression level may be determined by detecting the gene transcript in the patient-derived biological sample using techniques well known in the art. The gene transcripts detected by the present method include both the transcription and translation products, such as mRNA and protein.

For instance, the transcription product of the SYNGR4 gene can be detected by hybridization, e.g., Northern blot hybridization analyses, that use a SYNGR4 gene probe to the gene transcript. The detection may be carried out on a chip or an array. The use of an array is preferable for detecting the expression level of a plurality of genes including the SYNGR4 gene. As another example, amplification-based detection methods, such as reverse-transcription based polymerase chain reaction (RT-PCR) which use primers specific to the SYNGR4 gene may be employed for the detection (see Example). The SYNGR4 gene-specific probe or primers may be designed and prepared using conventional techniques by referring to the whole sequence of the SYNGR4 gene (SEQ ID NO: 13). For example, the primers (SEQ ID NOs: 1 and 2) used in the Example may be employed for the detection by RT-PCR, but the present invention is not restricted thereto.

Specifically, a probe or primer used for the present method hybridizes under stringent, moderately stringent, or low stringent conditions to the mRNA of the SYNGR4 gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degrees Centigrade for short probes or primers (e.g., 10 to 50 nucleotides) and at least about 60 degrees Centigrade for longer probes or primers. Stringent conditions may also be achieved with the addition of destabilizing agents, such as formamide.

Alternatively, the translation product may be detected for the assessment of the present invention. For example, the quantity of the SYNGR4 protein may be determined. A method for determining the quantity of the protein as the translation product includes immunoassay methods that use an antibody specifically recognizing the SYNGR4 protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the SYNGR4 protein. Methods to prepare these kinds of antibodies for the detection of proteins are well known in the art, and any method may be employed in the present invention to prepare such antibodies and equivalents thereof.

As another method to detect the expression level of the SYNGR4 gene based on its translation product, the intensity of staining may be observed via immunohistochemical analysis using an antibody against SYNGR4 protein. Namely, the observation of strong staining indicates increased presence of the SYNGR4 protein and at the same time high expression level of the SYNGR4 gene.

Furthermore, the SYNGR4 protein is known to have a cell proliferating activity. Therefore, the expression level of the SYNGR4 gene can be determined using such cell proliferating activity as an index. For example, cells which express SYNGR4 are prepared and cultured in the presence of a biological sample, and then by detecting the extent of proliferation in a predetermined time period, or by measuring the cell cycle or the colony forming ability the cell proliferating activity of the biological sample can be determined.

Moreover, in addition to the expression level of the SYNGR4 gene, the expression level of other lung cancer-associated genes, for example, genes known to be differentially expressed in lung cancer may also be determined to improve the accuracy of the assessment. Examples of such other lung cell-associated genes include those described herein and in WO 2004/031413 and WO 2005/090603, the contents of which are incorporated by reference herein.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

The patient to be assessed for the prognosis of cancer according to the method is preferably a mammal and includes human, non-human primate, mouse, rat, dog, cat, horse, and cow.

A kit for diagnosing cancer or assessing the prognosis of cancer:

The present invention provides a kit for diagnosing cancer or assessing the prognosis of cancer. Preferably, the cancer is lung cancer. Specifically, the kit includes at least one reagent for detecting the expression of the SYNGR4 gene in a patient-derived biological sample, which reagent may be selected from the group of:

(a) a reagent for detecting mRNA of the SYNGR4 gene;

(b) a reagent for detecting the SYNGR4 protein; and

(c) a reagent for detecting the biological activity of the SYNGR4 protein.

Suitable reagents for detecting mRNA of the SYNGR4 gene include nucleic acids that specifically bind to or identify the SYNGR4 mRNA, including oligonucleotides which have a complementary sequence to a part of the SYNGR4 mRNA. These kinds of oligonucleotides are exemplified by primers and probes that are specific to the SYNGR4 mRNA. These kinds of oligonucleotides may be prepared based on methods well known in the art. If desired, the reagent for detecting the SYNGR4 mRNA may be immobilized on a solid support, e.g., a bead, an array chip, a porous strip, etc. Moreover, more than one reagent for detecting the SYNGR4 mRNA may be included in the kit.

Suitable reagents for detecting the SYNGR4 protein include antibodies to the SYNGR4 protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the SYNGR4 protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the SYNGR4 protein may be included in the kit.

Furthermore, the biological activity can be determined by, for example, measuring the cell proliferating activity due to the expressed SYNGR4 protein in the biological sample. For example, the cell is cultured in the presence of a patient-derived biological sample, and then by detecting the speed of proliferation, or by measuring the cell cycle or the colony forming ability the cell proliferating activity of the biological sample can be determined in the presence and absence of expression of the SYNGR4 protein. If needed, the reagent for detecting the SYNGR4 mRNA may be immobilized on a solid support. Moreover, more than one reagent for detecting the biological activity of the SYNGR4 protein may be included in the kit.

The kit may contain more than one of the aforementioned reagents. Furthermore, the kit may include a solid support and reagent for binding a probe against the SYNGR4 gene or antibody against the SYNGR4 protein, a medium and container for culturing cells, positive and negative control reagents, and a secondary antibody for detecting an antibody against the SYNGR4 protein. For example, tissue samples obtained from patient with good prognosis or poor prognosis may serve as useful control reagents. A kit of the present invention may further include other materials desirable from a commercial end user standpoint, including buffers, diluents, filters, needles, syringes, and package inserts (e.g., written, tape, CD-ROM, etc.) with instructions for use. These reagents and such may be included in a container with a label. Suitable containers include bottles, vials, and test tubes. The containers may be formed from a variety of materials, such as glass or plastic.

As an embodiment of the present invention, when the reagent is a probe against the SYNGR4 mRNA, the reagent may be immobilized on a solid support, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of SYNGR4 mRNA present in the sample. The detection sites may be configured in any suitably detectable shape and are typically in the shape of a bar or dot spanning the width of a test strip.

The kit of the present invention may further include a positive control sample or SYNGR4 standard sample. The positive control sample of the present invention may be prepared by collecting SYNGR4 positive blood samples and then those SYNGR4 levels are assayed. Alternatively, purified SYNGR4 protein or polynucleotide may be added to SYNGR4 free serum to form the positive sample or the SYNGR4 standard.

Screening for an Anti-Lung Cancer Compounds

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic macromolecular compounds (including nucleic acid constructs, such as antisense RNA, siRNA, Ribozymes, and aptamer etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (US Pat. No. 5,223,409), spores (US Pat. No. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or a partial amino acid sequence of the obtained protein may be analyzed to prepare an oligo DNA as a probe based on the sequence, and screen cDNA libraries with the probe to obtain a DNA encoding the protein. The obtained DNA is confirmed for its usefulness in preparing the test agent which is a candidate for treating or preventing cancer.

Test agents useful in the screenings described herein can also be antibodies that specifically bind to SYNGR4 protein or partial peptides thereof that lack the biological activity of the original proteins in vivo.

Although the construction of test agent libraries is well known in the art, herein below, additional guidance in identifying test agents and construction libraries of such agents for the present screening methods are provided.

(i) Molecular Modeling:

Construction of test agent libraries is facilitated by knowledge of the molecular structure of compounds known to have the properties sought, and/or the molecular structure of SYNGR4. One approach to preliminary screening of test agents suitable for further evaluation is computer modeling of the interaction between the test agent and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. Computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents treating or preventing the lung cancer.

(ii) Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995-2009 Wiley Interscience; Sambrook et al., Molecular Cloning: A Laboratory Manual, 3rd Ed, 2001, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31.; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

(iii) Other Candidates:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Aptamers are macromolecules composed of nucleic acid that bind tightly to a specific molecular target. Tuerk and Gold (Science. 249:505-510 (1990)) discloses SELEX (Systematic Evolution of Ligands by Exponential Enrichment) method for selection of aptamers. In the SELEX method, a large library of nucleic acid molecules (e.g., 10¹⁵ different molecules) can be used for screening.

Screening for an SYNGR4 Binding Compound

In present invention, over-expression of SYNGR4 was detected in lung cancer, where no expression of SYNGR4 was observed in normal organs (FIGS. 1 and 2). Therefore, using the SYNGR4 genes, proteins encoded by the genes, the present invention provides a method of screening for a compound that binds to SYNGR4. Due to the expression of SYNGR4 in lung cancer, a compound binds to SYNGR4 is expected to suppress the proliferation of lung cancer cells, and thus be useful for treating or preventing lung cancer. Therefore, the present invention also provides a method for screening a compound that suppresses the proliferation of lung cancer cells, and a method for screening a compound for treating or preventing lung cancer using the SYNGR4 polypeptide. Specially, an embodiment of this screening method includes the steps of:

(a) contacting a test compound with a polypeptide encoded by a polynucleotide of SYNGR4;

(b) detecting the binding activity between the polypeptide and the test compound; and

(c) selecting the test compound that binds to the polypeptide.

In the present invention, it is revealed that suppressing the expression of SYNGR4, reduces lung cancer cell growth. Thus, by screening for candidate compounds that binds to the SYNGR4 polypeptide, candidate compounds that find use to treat or prevent lung cancers can be identified. The usefulness of the candidate compounds to treat or prevent lung cancers may be evaluated by a secondary and/or further screening to identify therapeutic agents for lung cancers.

According to the present invention, the therapeutic effect of the test agent or compound on inhibiting the lung cancer cell growth or a candidate agent or compound for treating or preventing a disease that is in part caused or promoted by SYNGR4 expression (“SYNGR4 associating disease”) may be evaluated. Therefore, the present invention also provides a method of screening for a candidate agent or compound for inhibiting the cell growth or a candidate agent or compound for treating or preventing SYNGR4 associating disease, using the SYNGR4 polypeptide or fragments thereof including the steps as follows:

a) contacting a test agent or compound with the SYNGR4 polypeptide or a functional fragment thereof;

b) detecting the binding activity between the polypeptide or a functional fragment thereof, and the test compound, and

c) correlating the binding activity of b) with the therapeutic effect of the test agent or compound.

In the present invention, the therapeutic effect may be correlated with the binding activity to SYNGR4 polypeptide or a functional fragment thereof. For example, when the test agent or compound bind to SYNGR4 polypeptide or a functional fragment thereof, the test agent or compound may be identified or selected as the candidate agent or compound having the therapeutic effect. Alternatively, when the test agent or compound does not bind to SYNGR4 polypeptide or a functional fragment thereof, the test agent or compound may be identified as the agent or compound having no significant therapeutic effect.

The screening methods of the present invention will be described in more detail below.

The SYNGR4 polypeptide to be used for screening may be a recombinant polypeptide or a protein derived from nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the SYNGR4 polypeptide using the SYNGR4 polypeptide, any method known in the art can be used. Such a screening can be conducted by, for example, immunoprecipitation method, specifically, in the following manner. The gene encoding the SYNGR4 polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by SYNGR4 gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the SYNGR4 polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the SYNGR4 polypeptide (Experimental Medicine 13: 85-90 (1995)).

In immunoprecipitation, an immune complex is formed by adding these antibodies to cell lysate prepared using an appropriate detergent. The immune complex consists of the SYNGR4 polypeptide, a polypeptide including the binding ability with the polypeptide, and an antibody. Immunoprecipitation can be also conducted using antibodies against the SYNGR4 polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by SYNGR4 gene is prepared as a fusion protein with an epitope, such as GST, an immune complex can be formed in the same manner as in the use of the antibody against the SYNGR4 polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the SYNGR4 polypeptide is difficult to detect by a common staining method, such as Coomassie staining or silver staining, the detection sensitivity for the protein can be improved by culturing cells in culture medium containing radioactive isotope, ³⁵S-methionine or ³⁵5-cysteine, labeling proteins in the cells, and detecting the proteins. The target protein can be purified directly from the SDS-polyacrylamide gel and its sequence can be determined, when the molecular weight of a protein has been revealed.

As a method of screening for proteins binding to the SYNGR4 polypeptide using the polypeptide, for example, West-Western blotting analysis (Skolnik et al., Cell 65: 83-90 (1991)) can be used. Specifically, a protein binding to the SYNGR4 polypeptide can be obtained by preparing a cDNA library from cultured cells (e.g., LC176, LC319, A549, NCI-H23, NCI-H226, NCI-H522, PC3, PC9, PC14, SK-LU-1, EBC-1, RERF-LC-AI, SK-MES-1, SW900, and SW1573) expected to express a protein binding to the SYNGR4 polypeptide using a phage vector (e.g., ZAP), expressing the protein on LB-agarose, fixing the protein expressed on a filter, reacting the purified and labeled SYNGR4 polypeptide with the above filter, and detecting the plaques expressing proteins bound to the SYNGR4 polypeptide according to the label. The polypeptide of the invention may be labeled by utilizing the binding between biotin and avidin, or by utilizing an antibody that specifically binds to the SYNGR4, or a peptide or polypeptide (for example, GST) that is fused to the SYNGR4 polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the SYNGR4 polypeptide is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HTS3 gene.

A compound binding to the polypeptide encoded by SYNGR4 gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a means for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

Methods of screening for molecules that bind when the immobilized SYNGR4 polypeptide is exposed to synthetic chemical compounds, or natural substance banks or a random phage peptide display library, and methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the SYNGR4 protein (including agonist and antagonist) are well known to one skilled in the alt.

Screening for a Compound Suppressing the Biological Activity of SYNGR4

In the present invention, the SYNGR4 protein has the activity of promoting cell proliferation of lung cancer cells (FIG. 3B), and cell invasion activity (FIG. 4A). Using these biological activities, the present invention provides a method for screening a compound that suppresses the proliferation of lung cancer cells, and a method for screening a compound for treating or preventing lung cancer. Thus, the present invention provides a method of screening for a compound for treating or preventing lung cancer using the polypeptide encoded by the SYNGR4 gene including the steps as follows:

(a) contacting a test compound with a polypeptide encoded by a polynucleotide of SYNGR4;

(b) detecting the biological activity of the polypeptide of step (a); and

(c) selecting the test compound that suppresses the biological activity of the polypeptide encoded by the polynucleotide of SYNGR4 as compared to the biological activity of said polypeptide detected in the absence of the test compound.

In the present invention, it is revealed that suppressing the expression of SYNGR4, reduces lung cancer cell growth. Thus, by screening for candidate compounds that inhibit the biological activity of SYNGR4 polypeptide, candidate compounds that find use to treat or prevent lung cancers can be identified. The potential of these candidate compounds to treat or prevent cancers may be evaluated by a secondary and/or further screening to identify therapeutic agents useful in treating or preventing lung cancers. For example, when a compound binding to SYNGR4 protein inhibits, e.g., the proliferative or invasive activities of the lung cancer cells, it may be concluded that such compound has the SYNGR4 specific therapeutic effect.

According to the present invention, the therapeutic effect of the test agent or compound on inhibiting the cell growth or a candidate agent or compound for treating or preventing SYNGR4 associating disease may be evaluated. Therefore, the present invention also provides a method of screening for a candidate agent or compound for inhibiting the cell growth or a candidate agent or compound for treating or preventing SYNGR4 associating disease, using the SYNGR4 polypeptide or fragments thereof including the steps as follows:

a) contacting a test agent or compound with the SYNGR4 polypeptide or a functional fragment thereof; and

b) detecting the biological activity of the polypeptide or fragment of step (a), and

c) correlating the biological activity of b) with the therapeutic effect of the test agent or compound.

In the present invention, the therapeutic effect may be correlated with the biological activity SYNGR4 polypeptide or a functional fragment thereof. For example, when the test agent or compound suppresses or inhibits the biological activity SYNGR4 polypeptide or a functional fragment thereof as compared to a level detected in the absence of the test agent or compound, the test agent or compound may be identified or selected as the candidate agent or compound having the therapeutic effect. Alternatively, when the test agent or compound does not suppress or inhibit the biological activity SYNGR4 polypeptide or a functional fragment thereof as compared to a level detected in the absence of the test agent or compound, the test agent or compound may be identified as the agent or compound having no significant therapeutic effect. The methods of the present invention will be described in more detail below. Any SYNGR4 polypeptides can be used for screening so long as they include the biological activity of the SYNGR4 protein. Such biological activity includes cell-proliferating activity or invasive activity of the SYNGR4 protein. For example, SYNGR4 protein can be used and polypeptides functionally equivalent to these proteins can also be used. Such polypeptides may be expressed endogenously or exogenously by cells. Further SYNGR4 protein has interacting activity with GRB2, and tyrosine-46 residue of SYNGR4 is indispensable for the activity. SYNGR4 could exert oncogenic function possibly with GRB2-PAK1 and subsequent MAPK signal activation.

The compound isolated by this screening is a candidate for antagonists of the polypeptide encoded by SYNGR4 gene. The term “antagonist” refers to molecules that inhibit the function of the polypeptide by binding thereto. Said term also refers to molecules that reduce or inhibit expression of the gene encoding SYNGR4. Moreover, a compound isolated by this screening is a candidate for compounds which inhibit the in vivo interaction of the SYNGR4 polypeptide with molecules (including DNAs and proteins).

When the biological activity to be detected in the present method is cell proliferation, it can be detected, for example, by preparing cells which express the SYNGR4 polypeptide, culturing the cells in the presence of a test compound, and determining the speed of cell proliferation, measuring the cell cycle and such, as well as by measuring survival cells or the colony forming activity, for example, shown in FIG. 3. The compounds that reduce the speed of proliferation of the cells expressed SYNGR4 are selected as candidate compounds for treating or preventing lung cancer.

More specifically, the method includes the step of:

(a) contacting a test compound with cells overexpressing SYNGR4;

(b) measuring cell-proliferating activity; and

(c) selecting the test compound that reduces the cell-proliferating activity in the comparison with the cell-proliferating activity in the absence of the test compound.

In preferable embodiments, the method of the present invention may further include the steps of:

(d) selecting the test compound that has no effect on the cells that express little or no SYNGR4.

When the biological activity to be detected in the present method is invasive activity, it can be detected, for example, by preparing cells which express SYNGR4 polypeptide and counting invasive cells number using any method known in the art, e.g., using a matrigel invasion assay, for example, shown in FIG. 4A. The compounds that reduce the invasive cells number are selected as candidate compounds for treating or preventing lung cancer.

More specifically, the methods include the steps of:

(a) contacting a test compound with a cell that over-expresses SYNGR4;

(b) measuring the invasive activity of the cell; and

(c) selecting the test compound that reduces the invasive activity of the cell in the comparison with the invasive activity of the cell in the absence of the test compound.

In preferable embodiments, the method of the present invention may further include the steps of:

(d) selecting the test compound that has no effect on the cells that express little or no SYNGR4.

In the present invention, it is revealed that suppressing the expression of SYNGR4, reduces lung cancer cell invasion. Thus, by screening for candidate compounds that reduces the invasive activity, candidate compounds that find use to treat or prevent lung cancer cell invasion can be identified. Potential of these candidate compounds to treat or prevent lung cancer cell invasion may be evaluated by secondary and/or further screening to identify therapeutic agents for cancer invasion.

According to the present invention, the therapeutic effect of the test agent or compound on inhibiting the cancer cell invasion or a candidate agent or compound for treating or preventing lung cancer cell invasion may be evaluated. Therefore, the present invention also provides a method of screening for a candidate agent or compound for inhibiting lung cancer cell invasion or a candidate agent or compound for treating or preventing lung cancer cell invasion, using the SYNGR4 polypeptide or fragments thereof including the steps as follows:

a) contacting a test agent or compound with a cell expressing the SYNGR4 polypeptide or a functional fragment thereof;

b) measuring the invasive activity of the cell, and

c) correlating the invasive activity of the cell of b) with the therapeutic effect of the test agent or compound.

In the present invention, the therapeutic effect may be correlated with the invasive activity. For example, when the test agent or compound suppresses or inhibits the invasive activity as compared to a level detected in the absence of the test agent or compound, the test agent or compound may be identified or selected as the candidate agent or compound having the therapeutic effect. Alternatively, when the test agent or compound does not suppress or inhibit the invasive activity as compared to a level detected in the absence of the test agent or compound, the test agent or compound may be identified as the agent or compound having no significant therapeutic effect. “Suppress the biological activity” as defined herein refers to preferably at least 10% suppression of the biological activity of SYNGR4 in comparison with in absence of the compound, more preferably at least 25%, 50% or 75% suppression and most preferably at 90% suppression.

Screening for Compounds Altering the Expression of SYNGR4

In the present invention, the decrease of the expression of SYNGR4 by siRNA inhibits lung cancer cell proliferation (FIG. 3A). Therefore, the present invention provides a method of screening for a compound that inhibits the expression of SYNGR4. A compound that inhibits the expression of SYNGR4 is expected to suppress the proliferation of lung cancer cells, and thus is useful for treating or preventing lung cancer. Therefore, the present invention also provides a method for screening a compound that suppresses the proliferation of lung cancer cells, and a method for screening a compound for treating or preventing lung cancer. In the context of the present invention, such screening may include, for example, the following steps:

(a) contacting a candidate compound with a cell expressing SYNGR4; and

(b) selecting the candidate compound that reduces the expression level of SYNGR4 as compared to a control.

In the present invention, it is revealed that suppressing the expression of SYNGR4, reduces lung cancer cell growth. Thus, by screening for candidate compounds that inhibit the expression level of SYNGR4, candidate compounds that find use to treat or prevent cancers that are in part caused or promoted by the overexpression of SYNGR4 can be identified. The potential of these candidate compounds to treat or prevent cancers may be evaluated by secondary and/or further screening to identify therapeutic agents for SYNGR4-associated cancers.

According to the present invention, the therapeutic effect of the test agent or compound on inhibiting the cell growth or a candidate agent or compound for treating or preventing SYNGR4 associating disease may be evaluated. Therefore, the present invention also provides a method for screening a candidate agent or compound that suppresses the proliferation of cancer cells, and a method for screening a candidate agent or compound for treating or preventing SYNGR4 associating disease.

In the context of the present invention, such screening may include, for example, the following steps:

a) contacting a test agent or compound with a cell expressing the SYNGR4 gene;

b) detecting the expression level of the SYNGR4 gene; and

c) correlating the expression level of b) with the therapeutic effect of the test agent or compound.

In the present invention, the therapeutic effect may be correlated with the expression level of the SYNGR4 gene. For example, when the test agent or compound reduces the expression level of the SYNGR4 gene as compared to a level detected in the absence of the test agent or compound, the test agent or compound may be identified or selected as the candidate agent or compound having the therapeutic effect. Alternatively, when the test agent or compound does not reduce the expression level of the SYNGR4 gene as compared to a level detected in the absence of the test agent or compound, the test agent or compound may by identified as the agent or compound having no significant therapeutic effect.

The methods of the present invention will be described in more detail below.

Cells expressing the SYNGR4 include, for example, cell lines established from lung cancer; such cells can be used for the above screening of the present invention (e.g., A427, A549,LC319,PC14,PC3,PC9, NCI-H1373, NCI-H1781, NCI-H358, NCI-H226, NCI-H520, NCI-H1703, NCI-H2170, EBC-1, RERF-LC-AI, LX1, DMS114, DMS273, SBC-3, SBC-5, NCI-H196, NCI-H446). The expression level can be estimated by methods well known to one skilled in the art, for example, RT-PCR, Northern bolt assay, Western blot assay, immunostaining and flow cytometry analysis. “reduce the expression level” as defined herein are preferably at least a 10% reduction of expression level of SYNGR4 in comparison to the expression level in absence of the compound, more preferably at least a 25%, 50% or 75% reduced level and most preferably at least a 95% reduced level. The compounds of use are described herein, including chemical compounds, double-strand nucleotides, and so on. The preparation of the double-strand nucleotide is in aforementioned description. In the methods of screening, a compound that reduces the expression level of SYNGR4 are selected as candidate compounds to be used for the treatment or prevention of lung cancer.

Alternatively, the screening method of the present invention may include the following steps:

(a) contacting a candidate compound with a cell into which a vector, including the transcriptional regulatory region of SYNGR4 and a reporter gene that is expressed under the control of the transcriptional regulatory region, has been introduced;

(b) measuring the expression or activity of said reporter gene; and

(c) selecting the candidate compound that reduces the expression or activity of said reporter gene.

In the present invention, it is revealed that suppressing the expression of SYNGR4, reduces lung cancer cell growth. Thus, by screening for candidate compounds that inhibits the expression or activity of said reporter gene, candidate compounds find use to treat or prevent lung cancers can be identified. Potential of these candidate compounds to treat or prevent cancers may be evaluated by secondary and/or further screening to identify therapeutic agents for lung cancers.

According to the present invention, the therapeutic effect of the test agent or compound on inhibiting lung cancer cell growth or a candidate agent or compound for treating or preventing SYNGR4 associating disease may be evaluated. Therefore, the present invention also provides a method for screening a candidate agent or compound that suppresses the proliferation of lung cancer cells, and a method for screening a candidate agent or compound for treating or preventing SYNGR4 associating disease.

According to another aspect, the present invention provides a method which includes the following steps of:

a) contacting a test agent or compound with a cell into which a vector, composed of the transcriptional regulatory region of the SYNGR4 gene and a reporter gene that is expressed under the control of the transcriptional regulatory region has been introduced;

b) detecting the expression or activity of said reporter gene; and

c) correlating the expression level of b) with the therapeutic effect of the test agent or compound.

In the present invention, the therapeutic effect may be correlated with the expression or activity of said reporter gene. For example, when the test agent or compound reduces the expression or activity of said reporter gene as compared to a level detected in the absence of the test agent or compound, the test agent or compound may be identified or selected as the candidate agent or compound having the therapeutic effect. Alternatively, when the test agent or compound does not reduce the expression or activity of said reporter gene as compared to a level detected in the absence of the test agent or compound, the test agent or compound may be identified as an agent or compound having no significant therapeutic effect.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COST, HEK293, HeLa and so on. The reporter construct required for the screening can be prepared by connecting reporter gene sequence to the transcriptional regulatory region of SYNGR4. The transcriptional regulatory region of SYNGR4 herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “Reduces the expression or activity” as defined herein are preferably at least a 10% reduction of the expression or activity of the reporter gene in comparison with in absence of the compound, more preferably at least a 25%, 50% or 75% reduction and most preferably at least a 95% reduction.

Screening Using the Phosphorylation Level of SYNGR4 as Index

Furthermore, in the present invention, it was confirmed that the SYNGR4 proteins were phosphorylated. Thus, a compound that inhibits the phosphorylation of SYNGR4 protein can be screened using such modification as an index. Therefore, the present invention also provides a method for screening a compound for inhibits the phosphorylation of SYNGR4 protein. Furthermore, the present invention also provides a method for screening a compound for treating or preventing cancer. The method is particularly suited for screening agents that may be used in treating or preventing cancer.

More specifically, the method includes the steps of:

(a) contacting a cell that expresses a polypeptide selected from the group consisting of:

(1) a polypeptide including the amino acid sequence of SEQ ID NO: 14;

(2) a polypeptide that includes the amino acid sequence of SEQ ID NO: 14 in which one or more amino acids are substituted, deleted, inserted, and/or added and that has a biological activity equivalent to a protein consisting of the amino acid sequence of SEQ ID NO: 14

(3) a polypeptide that shares at least 90%, 93%, 95%, 96%, 97%, 98% or 99% sequence identity with a polypeptide including the amino acid sequence of SEQ ID NO: 14 wherein the polypeptide has a biological activity equivalent to a polypeptide of the amino acid sequence of SEQ ID NO: 14; and

(4) a polypeptide encoded by a polynucleotide that hybridizes under stringent conditions to a polynucleotide consisting of the nucleotide sequence of SEQ ID NO: 13, wherein the polypeptide has a biological activity equivalent to a polypeptide consisting of the amino acid sequence of SEQ ID NO: 14;

with a test compound;

(b) detecting the phosphorylation level of the polypeptide;

(c) comparing the phosphorylation level of the polypeptide with the phosphorylation level of the polypeptide detected in the absence of the compound; and

(d) selecting the compound that reduced the phosphorylation level of the polypeptide as an inhibitor of the phosphorylation of the polypeptide or a compound for treating or preventing cancer.

Herein, any cell may be used so long as it expresses the SYNGR4 polypeptide or functional equivalents thereof. The cell used in the present screening may be a cell naturally expressing the SYNGR4 polypeptide including, for example, cells derived from and cell-lines established from lung cancer and testis. Cell-lines of lung cancer such as A427, A549, LC319, PC-3, PC-9, PC-14, NCI-H1373, NCI-H1781, NCI-H358, NCI-H226, EBC-1, NCI-H520, NCI-H1703, NCI-H2170, RERF-LC-AI, DMS114, DMS273, SBC-3, SBC-5, NCI-H196, and NCI-H446 can be employed.

Alternatively, the cell used in the screening may be a cell that naturally does not express the SYNGR4 polypeptide and which is transfected with an SYNGR4 polypeptide- or an SYNGR4 functional equivalent-expressing vector. Such recombinant cells can be obtained through known genetic engineering methods (e.g., Morrison D A., J Bacteriology 1977, 132: 349-51; Clark-Curtiss & Curtiss, Methods in Enzymology (eds. Wu et al.) 1983, 101: 347-62) as mentioned above.

Any of the aforementioned test compounds may be used for the present screening. However, it is preferred to select compounds that can permeate into a cell. Alternatively, when the test compound is a polypeptide, the contact of a cell and the test agent in the present screening can be performed by transforming the cell with a vector that includes the nucleotide sequence coding for the test agent and expressing the test agent in the cell.

In another embodiment, conditions suitable for phosphorylation of SYNGR4 polypeptide or functional equivalents thereof can be provided in vitro. This screening method includes the steps of:

(a) contacting a test compound with the polypeptide of the present invention or fragment thereof (e.g. including tyrosine-46);

(b) detecting the phosphorylation of the polypeptide of step (a); and

(c) selecting a compound that suppresses the phosphorylation of the polypeptide in comparison with the biological activity detected in the absence of the test compound. In the present invention, as mentioned above, the biological activity of the SYNGR4 protein is preferably phosphorylated activity. The skilled artisan can estimate phosphorylation level.

Accordingly, in these embodiments, the present invention provides a method of screening an agent for inhibiting the phosphorylation of SYNGR4 or preventing or treating cancer including the steps of:

(a) contacting a polypeptide selected from the group consisting of:

(1) a polypeptide including the amino acid sequence of SEQ ID NO: 14;

(2) a polypeptide that includes the amino acid sequence of SEQ ID NO: 14 in which one or more amino acids are substituted, deleted, inserted, and/or added and that has a biological activity equivalent to a protein consisting of the amino acid sequence of SEQ ID NO: 14

(3) a polypeptide that shares at least 90%, 93%, 95%, 96%, 97%, 98% or 99% sequence identity with a polypeptide including the amino acid sequence of SEQ ID NO: 14 wherein the polypeptide has a biological activity equivalent to a polypeptide of the amino acid sequence of SEQ ID NO: 14; and

(4) a polypeptide encoded by a polynucleotide that hybridizes under stringent conditions to a polynucleotide consisting of the nucleotide sequence of SEQ ID NO: 13, wherein the polypeptide has a biological activity equivalent to a polypeptide consisting of the amino acid sequence of SEQ ID NO: 14; or a fragment thereof including a phosphorylation site

with a test compound under a condition that allows phosphorylation of the polypeptide;

(b) detecting the phosphorylation level of the polypeptide or the fragment thereof;

(c) comparing the phosphorylation level of the substrate with the phosphorylation level of the polypeptide detected in the absence of the test compound; and

(d) selecting the compound that reduced the phosphorylation level of the polypeptide as a compound for inhibiting the phosphorylation of the polypeptide or treating or preventing cancer.

In these embodiments, a condition that allows phosphorylation of SYNGR4 polypeptide can be provided by incubating the polypeptide with suitable kinase for phosphorylation the SYNGR4 polypeptide and ATP. In some embodiments, the SYNGR4 polypeptide is further contacted with an AURKB polypeptide. Further, in the preferable embodiments, a substance enhancing phosphorylation of the SYNGR4 polypeptide can be added to the reaction mixture of screening. When phosphorylation of the polypeptide is enhanced by the addition of the substance, the phosphorylation level can be determined with higher sensitivity.

The phosphorylation level of SYNGR4 polypeptide or functional equivalent thereof may be detected according to any method known in the art (e.g. see Examples).

Screening Using the Interaction of SYNGR4 as Index

Furthermore, the present inventors revealed that SYNGR4 interacts with GRB2 (FIG. 5). Accordingly, it is believed that the interaction of both polypeptides plays a crucial role in carcinogenesis or cell proliferation, in particular cell proliferation of cancer. Hence, it is intended to screen for a compound useful in treating or preventing cancer, that inhibits an interaction between an SYNGR4 polypeptide and a GRB2 polypeptide or a vice versa interaction. Thus, the present invention provides methods of screening for a compound for inhibiting an interaction between a SYNGR4 polypeptide and a GRB2 polypeptide. Furthermore, the present invention provides methods of screening for a compound for inhibiting a binding between a SYNGR4 polypeptide and a GRB2 polypeptide, or treating or preventing cancer. The methods include the steps of:

(a) contacting an GRB2 polypeptide or functional equivalent thereof with an SYNGR4 polypeptide or functional equivalent thereof in the presence of a test compound;

(b) detecting the binding between the polypeptides of step (a); and

(c) selecting the test compound that inhibits the binding between the GRB2 and SYNGR4 polypeptides.

In the context of the present invention, a functional equivalent of an SYNGR4 or GRB2 polypeptide is a polypeptide that has a biological activity equivalent to an SYNGR4 polypeptide (SEQ ID NO: 14) or GRB2 polypeptide (SEQ ID NO: 23 or 25), respectively (see Definition).

Screening for a compound that suppresses the phosphorylation activity of PAK1, c-Raf, MEK1/2 and ERK1/2

In the context of the present invention, it was confirmed that phosphorylation of PAK1 (Thr423), c-Raf (Ser338), MEK1 (Ser 298), MEK1/2 (Ser217/221) and ERK1/2 (Thr202/204) were decreased in SYNGR4 knockdown. Meanwhile, phosphorylated PAK1 (Thr423), c-Raf (Ser338), MEK1 (Ser 298), MEK1/2 (Ser217/221) and ERK1/2 (Thr202/204) were increased following SYNGR4 expression (FIG. 6). These findings indicate that PAK1, c-Raf, MEK1, MEK1/2 and ERK1/2 are down-stream effector molecules for phosphorylation signaling of SYNGR4 polypeptide which leads to cell proliferation. In the present invention, down-stream effector of SYNGR4 refers to molecule which is phosphorylated by SYNGR4 directly or indirectly manner. Thus, down-stream effector of SYNGR4 includes molecules phosphorylated during signaling pathway from SYNGR4. For example, according to the present invention, SYNGR4 enhances phosphorylation level of PAK1 which is one of down-stream effectors of SYNGR4. In addition, phosphorylation level of c-Raf, MEK1, MEK1/2 and ERK are increased following to the phosphorylation of SYNGR4. Thus, these molecules are also down-stream effectors of SYNGR4. Therefore, by using this activity as an index, the present invention provides a method for screening a compound that suppresses the proliferation of cancer cells expressing SYNGR4, GRB2 and PAK1, and c-Raf, MEK1/2 or ERK1/2, and a method of screening for a compound for treating or preventing cancer, particularly cancers including lung cancer. Thus, the present invention provides a method of screening for a compound for inhibiting the activity of SYNGR4 for phosphorylating down-stream effectors, or treating or preventing cancer using the polypeptide encoded by SYNGR4 gene including the steps as follows:

(a) contacting a test compound with a polypeptide encoded by a polynucleotide of SYNGR4 in the presence of polypeptides encoded by a polynucleotide GRB2 and PAK1 under the condition for phosphorylation of at least one of down-stream effector selected from the group consisting of PAK1, c-Raf, MEK1, MEK1/2 and ERK1/2,;

(b) detecting the phosphorylation level of the down-stream effector of SYNGR4; and

(c) selecting the test compound that suppresses the phosphorylation level of the down-stream effector of SYNGR4 as compared to the phosphorylation level of the down-stream effector of SYNGR4 detected in the absence of the test compound.

In preferred embodiments, the phosphorylation level of the down-stream effector of SYNGR4 to be detected is that of Thr423 of PAK1, Ser338 of c-Raf, Ser 298 of MEK1, Ser217/221 of MEK1/2, and Thr202/204 of ERK1/2, respectively. In the present invention, the condition for phosphorylation of at least one of down-stream effectors selected from the group consisting of PAK1, c-Raf, MEK1, MEK1/2 and ERK1/2 may be provided via culturing cells expressing SYNGR4 and at least one of the down-stream effectors thereof. For example, cells expressing SYNGR4 and all of these down-stream effectors including GRB2 are preferred condition for phosphorylation of these down-stream effectors. In particular, lung cancer cell lines expressing these molecules may be used for the present invention. Alternatively, any cells endogenously expressing the down-stream effectors transfected with vector for expressing SYNGR4 are also useful for present invention.

According to the present invention, the therapeutic effect of the test compound on suppressing the phosphorylation activity of PAK1, c-Raf, MEK1/2 or ERK1/2, or a candidate compound for treating or preventing cancer relating to SYNGR4 (e.g., lung cancer.) may be evaluated. Therefore, the present invention also provides a method of screening for a candidate compound for suppressing the phosphorylation activity, or a candidate compound for treating or preventing cancer relating to SYNGR4, using the SYNGR4 polypeptide or fragments thereof and PAK1, c-Raf, MEK1/2 or ERK1/2 polypeptide or fragments thereof including the steps as follows:

a) contacting a test compound with the SYNGR4 polypeptide or a functional fragment thereof in the presence of the GRB2 and PAK1, and c-Raf, MEK1/2 or ERK1/2 polypeptide or a functional fragment thereof, under the condition for phosphorylation of at least one of down-stream effectors of SYNGR4 selected from the group consisting of PAK1, c-Raf, MEK1, MEK1/2 and ERK1/2;

b) detecting the phosphorylation level of the down-stream effector of SYNGR4, and

c) correlating the phosphorylation level of b) with the therapeutic effect of the test agent or compound.

In the context of the present invention, the therapeutic effect may be correlated with the phosphorylating activity of PAK1, c-Raf, MEK1/2 or ERK1/2 polypeptide or a functional fragment thereof enhanced by SYNGR4. For example, when the test agent or compound suppresses or inhibits the phosphorylating activity of PAK1, c-Raf, MEK/2 or ERK1/2 polypeptide or a functional fragment thereof as compared to a level detected in the absence of the test agent or compound, the test agent or compound may identified or selected as the candidate agent or compound having the therapeutic effect. Alternatively, when the test agent or compound does not suppress or inhibit the phosphorylating activity of PAK1, c-Raf, MEK1/2 or ERK1/2 polypeptide or a functional fragment thereof as compared to a level detected in the absence of the test agent or compound, the test agent or compound may identified as the agent or compound having no significant therapeutic effect.

The method of the present invention will be described in more detail below.

Any polypeptides can be used for screening so long as they suppress an phosphorylating activity of PAK1, c-Raf, MEK1/2 or ERK1/2. For example, SYNGR4 protein and GRB2, PAK1, c-Raf, MEK1/2 or ERK1/2 protein can be used and polypeptides functionally equivalent to these proteins can also be used. Such polypeptides may be expressed endogenously or exogenously by cells.

The compound isolated by this screening is a candidate for antagonists of the polypeptide encoded by SYNGR4 gene. The term “antagonist” refers to molecules that inhibit the function of the polypeptide by binding thereto. This term also refers to molecules that reduce or inhibit expression of the gene encoding SYNGR4. Moreover, a compound isolated by this screening is a candidate for compounds which inhibit the in vivo interaction of the SYNGR4 polypeptide with GRB2.

When the biological activity to be detected in the present method is phosphorylating, it can be detected, for example, by preparing cells which express the SYNGR4, GRB2 and PAK1, and c-Raf, MEK1/2 or ERK1/2 polypeptide, culturing the cells in the presence of a test compound, and determining the phosphorylating of PAK1, c-Raf, MEK1/2 or ERK1/2, measuring the cell cycle and such, as well as by measuring survival cells or the colony forming activity. The compounds that reduce the phosphorylating of PAK1, and c-Raf, MEK1/2 or ERK1/2 of the cells expressed SYNGR4 are selected as candidate compound for treating or preventing cancer including lung cancer.

In the preferred embodiments, control cells which do not express SYNGR4 polypeptide are used. Accordingly, the present invention also provides a method of screening for a candidate substance for inhibiting the cell growth or a candidate substance for treating or preventing SYNGR4 associating disease, using the SYNGR4 polypeptide or fragments thereof including the steps as follows:

(a) contacting a test compound with cells over-expressing SYNGR4, GRB2 and PAK1, and c-Raf, MEK1/2 or ERK1/2;

(b) measuring the phosphorylating activity of PAK1 (Thr423), c-Raf (Ser338), MEK1 (Ser 298), MEK1/2 (Ser217/221) and ERK1/2 (Thr202/204); and

(c) selecting the test compound that reduces the phosphorylating activity in the comparison with the cell-proliferating activity in the absence of the test compound.

In preferable embodiments, the method of the present invention may further include the step of:

(d) selecting the test compound that have no effect to the cells no or little expressing SYNGR4.

Alternatively, according to the present invention, potential antagonist for SYNGR4 polypeptide may be evaluate on the ability to inhibit SYNGR4 mediated phosphorylation of down-stream effector molecules of SYNGR4. For example, any compounds that bind to SYNGR4 polypeptide may be potential antagonist for the polypeptide.

Such compound can be isolated by following method which includes the steps of:

i) contacting a test compound with SYNGR4 polypeptide,

ii) detecting the binding between the test compound and SYNGR4 polypeptide, and

iii) selecting the test compound that binds to the SYNGR4 polypeptide as the potential antagonist for SYNGR4 polypeptide.

The phrase “suppress or reduce the phosphorylating ” as defined herein are preferably at least 10% suppression of the biological activity of SYNGR4 in comparison with in absence of the compound, more preferably at least 25%, 50% or 75% suppression and most preferably at 90% suppression. Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

### Example 1

### General Methods

1. Cell Lines and Tissue Samples.

The 22 human lung cancer cell lines used in this example included nine adenocarcinomas (ADC; A427, A549, LC319, PC-3, PC-9, PC-14, NCI-H1373, NCI-H1781, and NCI-H358), one adenosquamous carcinoma (ASC; NCI-H226), five squamous cell carcinomas (SCC; EBC-1, NCI-H520, NCI-H1703, NCI-H2170, and RERF-LC-AT), one large cell carcinoma (LX1), and six small cell lung cancers (SCLC; DMS114, DMS273, SBC-3, SBC-5, NCI-H196, and NCI-H446). Human bronchial epithelial cells (BEAS-2B) and Human small airway epithelial cells (SAEC) were used as a control. All cells were grown in monolayer in appropriate medium supplemented with 10% FCS and maintained at 37 degrees C. in humidified air with 5% CO₂. Primary lung cancer samples had been obtained earlier. Clinical stage was judged according to the International Union Against Cancer TNM classification (Sobin L et al., 6^{th} ed. New York 2002). A total of 339 formalin-fixed samples of primary NSCLCs (stage I-IIIA) including 203 ADCs, 100 SCCs, 25 LCCs, 11 ASCs and adjacent normal lung tissues, had been obtained earlier along with clinicopathological data from patients undergoing surgery at Saitama Cancer Center (Saitama, Japan). The use of all clinical materials mentioned were approved by individual institutional Ethical Committees.

2. Semiquantitative Reverse Transcription-PCR.

A total of 3 micro-g aliquot of mRNA from each sample was reversely transcribed to single-stranded cDNAs using random primer (Roche Diagnostics, Basel, Switzerland) and SuperScript II (Invitrogen, Carlsbad, Calif.). Semiquantitative reverse transcription-PCR (RT-PCR) experiments were carried out with the following sets of synthesized primers specific to SYNGR4 or beta-actin (ACTB) specific primers as an internal control: SYNGR4, 5′-CAACAGCCCTGTGAACATGC-3′ (SEQ ID NO: 1) and 5′-ACCCTTCTGGAGGGAGGATTC-3′ (SEQ ID NO: 2); ACTB, 5′-GAGGTGATAGCATTGCTTTCG-3′ (SEQ ID NO: 3)and 5′-CAAGTCAGTGTACAGGTAAGC-3′ (SEQ ID NO: 4). PCRs were optimized for the number of cycles to ensure product intensity to be within the linear phase of amplification.

3. Northern Blot Analysis.

Human multiple tissue blots covering 16 tissues (BD Biosciences, Palo Alto, Calif.) were hybridized with an [alpha-³²P1-dCTP-labeled, 406-bp PCR product of SYNGR4 that was prepared as a probe using primers 5′-CGGCTACCAGAACAAGATGG-3′ (SEQ ID NO: 5) and 5′-GAAGCGCTTGTAAGGGACTG-3′ (SEQ ID NO: 6). Prehybridization, hybridization, and washing were done following the manufacturer's specifications. The blots were autoradiographed with intensifying screens at −80 degrees C. for 7 days.

4. Construction of SYNGR4 Expressing Vector.

The entire coding region of SYNGR4 was amplified by RT-PCR using the primer sets (5′-GGAATTCCAGACCGTGCATCATGCACATCCCCAAAAGCCTCCAG-3′ (SEQ ID NO: 7) and 5′-CCGCTCGAGCGGGTTGTCAGGCATCATAGCAAGC-3′ (SEQ ID NO: 8). The product was digested with EcoRI and XhoI, and cloned into appropriate sites of a pcDNA3.1-myc/His A(+) vector (Invitrogen) that contained c-myc-His epitope sequences (LDEESILKQEHHHHHH)(SEQ ID NO: 15) at the COOH-terminal of the SYNGR4 protein. The inventors also constructed expression vector using pCAGGSn-3Fc vector and pCAGGSn-3FH vector, which contained 3× Flag epitope sequences (DYKDHDGDYKDHDIDYKDDDDK) (SEQ ID NO: 16) at the NH₂-terminal of the SYNGR4 protein. Generation of mutant SYNGR4 in which Tyr46 was replaced with phenylalanine (Y46F) was performed by standard mutagenesis PCR (Suzuki C, et al. Cancer Res 2005; 65:11314-25.). The primer sets used for SYNGR4-Y46F were as follows; forward, 5′-CGACGGCTtCCAGAACAAG-3′ (SEQ ID NO: 17) and reverse, 5′-CTTGTTCTGGaAGCCGTCG-3′ (SEQ ID NO: 18) (small characters indicate nucleotides that were mutated). In brief, the SYNGR4 sequence was amplified by PCR using primer set of forward cloning primer and reverse Y46F primer or of forward Y46F primer and reverse cloning primer. Two amplified PCR products were purified and fused by performing mutagenesis PCR.

5. Immunocytochemical Analysis.

For analyses under permeabilized condition, cells were plated on glass coverslips (Becton Dickinson Labware, Franklin Lakes, N.J.), fixed with 4% paraformaldehyde, and permeabilized with 0.1% Triton X-100 in PBS for 5 min at room temperature. Nonspecific binding was blocked by Casblock (ZYMED, San Francisco, Calif.) for 10 min at room temperature. Cells were then incubated for 60 min at room temperature with 1.3 micro-g/ml of a goat polyclonal anti-human SYNGR4 antibody (Santa Cruz Biotechnology, Santa Cruz, Calif.) diluted in PBS containing 1% BSA. After being washed with PBS, the cells were stained by Alexa488-conjugated secondary antibody (lnvitrogen) for 60 min at room temperature. After another wash with PBS, each specimen was mounted with Vectashield (Vector Laboratories, Inc., Burlingame, Calif.) containing 4′,6-diamidino-2-phenylindole and visualized with Spectral Confocal Scanning Systems (TSC SP2 AOBS; Leica Microsystems, Wetzlar, Germany). To determine subcellular localization of SYNGR4, fixed cells were divided into the condition with or without permeabilization. After blocking and incubation with primary antibody cells were treated with acid glycine for 5 min to remove antibodies that bind cell surface. After acid glycine treatment, secondary antibody and 4′,6-diamidino-2-phenylindole were treated by normal procedure.

6. Flow Cytometric Analysis.

Lung cancer cells (2×10⁶ cells) were incubated with a goat anti-SYNGR4 antibody (5 micro-g/mL; Santa Cruz Biotechnology, Santa Cruz, Calif.) for detecting cell surface SYNGR4 or control goat IgG (5 micro-g/mL; R&D Systems, Inc.) at 4 degrees C. for 30 min. The cells were washed in PBS and then incubated with AlexaFluor 488-conjugated anti-goat IgG (Invitrogen, Carlsbad, Calif.) at 4 degrees C. for 30 min. The cells were washed in PBS and analyzed on a FACScan flow cytometer (Becton Dickinson Labware, Bedford, Mass.) and analyzed by ModFit software (Verity Software House, Inc., Topsham, Me.). Mean fluorescence intensity was calculated as a relative signal-intensity value, i.e., cells treated with anti-SYNGR4 antibody/cells treated with control goat IgG.

7. Immunohistochemistry and Tissue Microarray.

In the invention, the SYNGR4 protein in clinical samples that had been embedded in paraffin blocks was stained the sections in the following manner. Briefly, 20 micro-g/mL of primary antibody to SYNGR4 (Santa Cruz Biotechnology) were added to each slide after blocking of endogenous peroxidase and proteins, and the sections were incubated with HRP-labeled anti-goat IgG [Histofine Simple Stain MAX PO (G), Nichirei, Tokyo, Japan] as the secondary antibody. Substrate-chromogen was added, and the specimens were counterstained with hematoxylin. Antigen blocking assays to examine antibody specificity to SYNGR4 was performed as follows. Before immunohistochemical staining, 20 micro-g/mL anti-SYNGR4 antibody (Catalog No.sc-34968; Santa Cruz Biotechnology) was incubated with SYNGR4 antigen peptide (Catalog No.sc-34968P; Santa Cruz Biotechnology) for 60 min at 37 degrees C. and the reaction product was centrifuged at 12,000×g for 15 min at 4 degrees C. to remove the immune complexes. The supernatant was used as a neutralized antibody for further analysis. Reacting mole ratio of anti-SYNGR4 antibody and its antigen peptide was 1:8.

Tumor tissue microarrays were constructed with formalin-fixed 339 primary lung cancers as described elsewhere (Chin S F et al., Mol Pathol 2003, 56: 275-9; Callagy G et al., Diagn Mol Pathol 2003, 12: 27-34; Callagy G et al., J Pathol 2005, 205: 388-96). The tissue area for sampling was selected based on visual alignment with the corresponding H&E-stained section on a slide. Three, four, or five tissue cores (diameter, 0.6 mm; depth, 3-4 mm) taken from a donor tumor block were placed into a recipient paraffin block with a tissue microarrayer (Beecher Instruments, Sun Prairie, Wis.). A core of normal tissue was punched from each case, and 5-micrometer sections of the resulting microarray block were used for immunohistochemical analysis. Three independent investigators semiquantitatively assessed SYNGR4 positivity without prior knowledge of clinicopathologic data. The intensity of SYNGR4 staining was evaluated using the following criteria: strong positive (scored as 2+), brown staining in >50% of tumor cells completely obscuring cytoplasm; weak positive (1+), any lesser degree of brown staining appreciable in tumor cell cytoplasm; and absent (scored as 0), no appreciable staining in tumor cells. Cases were accepted as strongly positive only if reviewers independently defined them as such.

8. Statistical Analysis.

Statistical analyses were done using the StatView statistical program (SAS, Cary, N.C.). Tumor-specific survival curves were calculated from the date of surgery to the time of death related to NSCLC or to the last follow-up observation. Kaplan-Meier curves were calculated for each relevant variable and for SYNGR4 expression; differences in survival times among patient subgroups were analyzed using the log-rank test. Univariate and multivariate analyses were done with the Cox proportional hazard regression model to determine associations between clinicopathologic variables and cancer-related mortality. First, it was analyzed associations between death and other prognostic factors, including age, gender, pathologic tumor classification, and pathologic node classification, taking into consideration one factor at a time. Second, multivariate Cox analysis was applied on backward (stepwise) procedures that always forced strong SYNGR4 expression into the model, along with any and all variables that satisfied an entry level of a P value of <0.05. As the model continued to add factors, independent factors did not exceed an exit level of P<0.05.

9. RNA Interference Assay.

The invention had previously established a vector-based RNA interference system, psiH1BX3.0 that was designed to synthesize small interfering RNAs (siRNA) in mammalian cells (Suzuki C et al., Cancer Res 2003, 63: 7038-41). Ten micrograms of siRNA expression vector were transfected using 30 micro-L of LipofectAMINE 2000 (Invitrogen) into lung cancer cell lines SBC-5 and A549. The transfected cells were cultured for 7 days in the presence of appropriate concentrations of geneticin (G418); the number of colonies was counted by Giemsa staining; and viability of cells was evaluated by 3-(4,5-dimethylthiazol-2-yl)-2,5-diphenyltetrazolium bromide assay at 7 days after the treatment. Briefly, cell counting kit-8 solution (Dojindo) was added to each dish at a concentration of 1/10 volume, and the plates were incubated at 37 degrees C. for additional 1 h. Absorbance was then measured at 490 nm, and at 630 nm as a reference, with a Microplate Reader 550 (Bio-Rad). To confirm suppression of SYNGR4 mRNA expression, semiquantitative RT-PCR experiments were carried out with the following synthesized SYNGR4-specific primers according to the standard protocol. The target sequences of the synthetic oligonucleotides for RNA interference were as follows: control 1 (EGFP: enhanced green fluorescent protein gene, a mutant of Aequorea victoria green fluorescent protein), 5′-GAAGCAGCACGACTTCTTC-3′ (SEQ ID NO: 9); control 2 (luciferase/LUC: Photinus pyralis luciferase gene), 5′-CGTACGCGGAATACTTCGA-3′ (SEQ ID NO: 10); siRNA-SYNGR4-#1, 5′-CAAGATGGAGTCTCCGCAG-3′ (SEQ ID NO: 11); siRNA-SYNGR4-#2, 5′-ATGATGCTCCAGTCCCTTA-3′ (SEQ ID NO: 12), siRNA-SYNGR4-#3, 5′-CGCAUUGCCGGCACCCGCU-3′ (SEQ ID NO: 19), siRNA-SYNGR4-#4, 5′-GCAUUGCCGGCACCCGCUU-3′ (SEQ ID NO: 20), siRNA-PAK1, 5′-CAAACAUUGUGAAUUACUU-3′ (SEQ ID NO: 21). The sense strand of the siRNA constructs were added TT at 3′.

10, Cell Growth Assays.

COS-7 cells transfected either with plasmids expressing SYNGR4 or with mock plasmids were seeded onto six-well plates (5×10⁴ cells/well), and maintained in medium containing 10% FBS and 0.4 mg/ml geneticin. After 72 hours cell proliferation was evaluated by the MTT assay using Cell Counting Kits (Wako, Osaka, Japan).

11.Matrigel Invasion Assay.

COS-7 and NIH3T3 cells transfected either with plasmids expressing SYNGR4 or with mock plasmids were grown to near confluence in DMEM containing 10% FBS. The cells were harvested by trypsinization, washed in DMEM without addition of serum or proteinase inhibitor, and suspended in DMEM at 2× W/mL. Before preparing the cell suspension, the dried layer of Matrigel matrix (Becton Dickinson Labware) was rehydrated with DMEM for 2 h at room temperature. DMEM (0.75 mL) containing 10% FBS was added to each lower chamber in 24-well Matrigel invasion chambers, and 0.5 mL (1×10⁵/cells) of cell suspension were added to each insert of the upper chamber. The plates of inserts were incubated for 22 h at 37 degrees C. After incubation, the chambers were processed; cells invading through the Matrigel were fixed and stained by Giemsa as directed by the supplier (Becton Dickinson Labware).

12. Antibody Treatment Assay Suppressing the Cell Invasive Activity of SYNGR4.

To assess the inhibitory effect of anti-SYNGR4 antibody on the invasive ability of mammalian cells that overexpressed exogenous or endogenous SYNGR4, Matrigel invasion assay was performed under the treatment with anti-SYNGR4 antibody (Santa Cruz Biotechnology). COS-7 cells transfected either with plasmids expressing SYNGR4 or with mock plasmids, or lung cancer cells expressing endogenous SYNGR4 were grown to near confluence in DMEM containing 10% FBS. The cells were harvested by tripsinization, washed in DMEM without FBS, and harvested into Matrigel chambers at a number of 1×10⁵ cells/chamber with 100 nM or 200 nM of anti-SYNGR4 antibody for COS-7 expressing SYNGR4, COS-7 transfected with mock, or lung cancer cells expressing SYNGR4 endogenously. These cells were also treated with 200 nM Isotype goat IgG or PBS as control assays. At lower chamber DMEM (0.75 mL) containing 10% FBS was added to each lower chamber in 24-well Matrigel invasion chambers, and the same concentration of anti-SYNGR4 antibody, Isotype IgG, or PBS as upper chamber was added to lower chamers. The plates of inserts were incubated for 22 h at 37 degrees C. and after incubation the chambers were processed; cells invading through the Matrigel were fixed and stained by Giemsa and number of invading cells were counted.

13. Antibodies and Reagent.

Anti-SYNGR4 antibody (Catalog No.sc-34968), anti-myc, and anti-GAPDH antibody were purchased from Santa Cruz Biotechnology. Anti-phospho ERK1/2 (Thr202/Tyr204), anti-ERK1/2, anti-phospho MEK1/2 (Ser217/221), anti-phospho MEK1 (Ser298), anti-MEK1/2, anti-phospho c-Raf (Ser338), anti-c-Raf, anti-phospho AKT (Thr308), anti-phospho AKT (Ser473), anti-AKT, anti-GRB2, anti-PAK1, and anti-phospho PAK1/2 (Thr423) antibodies were purchased from Cell signaling biotechnology. Anti-Flag M2 antibody was obtained from Sigma-Aldrich. Anti-phospho trypsin antibody was from Millipore. Isotype goat IgG used for flow cytometry was from R & D systems, Inc. Rac and Ras activation assay kits were purchased from Cell Biolabs, Inc and assays were performed according to the manufacturer's protocol.

14. Western Blot Analysis.

Lysates of A549 and SBC-3 cells transfected with si-SYNGR4-#1 or si-EGFP, and of COS-7 cells transfected either with plasmids expressing wild type or mutant SYNGR4-Y46F, or with mock plasmids, were subjected to western blotting. In brief, cells were incubated in 1 mL of lysis buffer (0.5% NP-40, 50 mmol/L Tris-HCl, 150 mmol NaCl) in the presence of protease inhibitor (Protease Inhibitor Cocktail Set III; Calbiochem). Western blotting was done using an ECL western-blotting analysis system (GE Healthcare Bio-sciences), as previously described (Suzuki C, et al. Cancer Res 2005;65:11314-25.). For the analyses of MAPK signaling activation by SYNGR4, specific antibodies for each MAPK signaling proteins (see above) were used, and a goat anti-mouse and -rabbit IgG-HRP antibody (GE Healthcare Bio-sciences) were served as the secondary antibodies for these experiments.

15. Phosphatase Assay.

COS-7 cells transfected either with plasmids expressing SYNGR4 plasmids were lysed by lysis buffer and were treated for 1 h at 30 degrees C. with 400 units of lambda-phosphatase (New England Biolabs) in phosphatase buffer containing 50 mmol/L Tris-HCL (pH 7.5), 0.1 mmol/L Na_{z}-EDTA, 5 mmol/L dithiothreitol, 2 mmol/L MgCl₂ and 0.01% Brij-35, followed by western blotting as described above.

16. Immunoprecipitation.

Cell lysates of COS-7 cells transfected either with plasmids expressing SYNGR4 or with mock plasmids were subjected to immunoprecipitation and western blotting. In Brief, cells were incubated in 1 mL lysis buffer (0.5% NP-40, 50 mmol/L Tris-HCl, 150 mmol NaCl) in the presence of protease inhibitor (Protease Inhibitor Cocktail Set III; Calbiochem Darmstadt). Cell extracts were precleared by incubation at 4 degrees C. for 1 h with 30 mL protein G-Agarose beads (Invitrogen), in final volumes of 1 ml lysis buffer in the presence of protease inhibitor. Immunoprecipitation and subsequent western blotting were done using antibodies specific for exogenous SYNGR4 (anti-myc antibody or anti-Flag antibody) and endogenous GRB2 or phosphorylated tyrosine.

Example 2

### SYNGR4 Expression in Lung Cancers and Normal Tissues

To identify novel molecules that can be applicable to develop novel biomarkers and treatments based on the biological characteristics of cancer cells, it was done genome-wide expression profile analysis of 101 lung carcinomas using a cDNA microarray (Kikuchi T et al., Oncogene 2003, 22: 2192-205; Kakiuchi S et al., Mol cancer Res 2003, 1: 485-99; Kakiuchi S et al., Hum Mol Genet 2004, 13: 3029-43; Kikuchi T et al., Int J Oncol 2006, 28: 799-805; Taniwaki M et al., Int J Oncol 2006, 29: 567-75). Among 32,256 genes screened, it was identified elevated expression (5-fold or higher) of EBI3 transcript in cancer cells in the great majority of the lung cancer samples examined. It was confirmed that its overexpression by means of semiquantitative RT-PCR experiments in 10 of 15 lung cancer tissues, in 17 of 22 lung cancer cell lines (FIG. 1A). The present inventors did immunofluorescence analysis to examine the subcellular localization of endogenous SYNGR4 in lung cancer cells. SYNGR4 was detected mainly at cytoplasm and on the surface of tumor cells at a high level in LC319, NCI-H1373, and A549 cells in which SYNGR4 transcript was detected by semiquantitative RT-PCR experiments, but not in NCI-H1781 cells as well as bronchial epithelia derived BEAS-2B and SAEC cells, these showed no expression of SYNGR4 gene (FIG. 1B). The results also indicated the specificity of SYNGR4 antibody. SYNGR4 was predicted to encode cell surface protein with four transmembrane domains, however there was no report that indicated whether both N-terminus and C-terminus of SYNGR4 could correspond to intra- or extracellular portion. Therefore, the present inventors first performed immunocytochemical analysis with or without a cell permeabilizing agent like Triton-X. Plasmids designed to express SYNGR4 with myc/His tag at C-terminus (pcDNA3.1-SYNGR4-myc/His) and those with 3× Flag-tag at N-terminus (pCAGGSn3F-SYNGR4) were constructed. Then, the plasmids or mock plasmids was transfected into COS-7 cells and stained the cells using anti-myc antibody for detecting myc-tagged SYNGR4 and anti-Flag antibody for Flag-tagged SYNGR4. It was confirmed that the expression of SYNGR4 protein on cell surface and in cytoplasm by immunocytochemical staining of the cells pretreated with Triton-X. The only cell surface staining of SYNGR4 protein was observed without Triton-X treatment (FIG. 1C, left top panels), indicating that C-terminus of SYNGR4 protein could be an extracellular portion. It was also confirmed that SYNGR4 was stained on cell surface of COS-7 cells transfected with N-terminus-tagged SYNGR4 expressing vector (data not shown). Since anti-SYNGR4 antibody recognizes C-terminus of SYNGR4, it was performed immunofluorescence analysis of lung cancer LC319 cells without Triton-X treatment and was confirmed that C-terminus of endogenous SYNGR4 protein was located extracellularly (FIG. 1C, left bottom panels). To further confirm that C-terminus and N-terminus of SYNGR4 are both extracellular portion, it was performed immunofluorescence analysis by treating the cells with the acid glycine after primary antibody reaction. Expectedly, SYNGR4 staining on the cell surface of SYNGR4-positive COS-7 cells LC319 cells disappeared by stripping the antibodies with acid glycine treatment (FIG. 1C, right panels). It was also measured the levels of SYNGR4 protein on the surface of SYNGR4-positive and -negative lung cancer cells by flow cytometry using the same anti-SYNGR4 antibody recognizing C-terminus of SYNGR4, and confirmed that both C-and N-terminus of SYNGR4 were detected on cell surface, and that the level of membrane SYNGR4 protein was correlated with the expression levels of SYNGR4 gene detected by semiquantitative RT-PCR (FIG. 1D).

Northern blot analysis using a SYNGR4 cDNA fragment as a probe identified a transcript of 1.2 kb only in testis, but not in any other normal tissues (FIG. 2A). The invention also examined expression of SYNGR4 protein with polyclonal antibody specific to SYNGR4 on five normal tissues (liver, heart, kidney, lung, and testis) and lung cancer tissues (ADC, SCC, and SCLC). SYNGR4 staining was mainly observed in cytoplasm of lung tumor cells and testis, but not detected in other four normal tissues (FIG. 2B).

Example 3

### Association of SYNGR4 Expression with Poor Prognosis for NSCLC Patients

To investigate the biological and clinicopathological significance of SYNGR4 in pulmonary carcinogenesis, it was carried out immunohistochemical staining on tissue microarray containing tissue sections from 339 NSCLC cases that underwent curative surgical resection. SYNGR4 staining detected with polyclonal antibody specific to SYNGR4 was mainly observed at membrane and cytoplasm of tumor cells but was not in normal lung cells (FIG. 2C, left panels). This invention classified a pattern of SYNGR4 expression on the tissue array ranging from absent (scored as 0) to weak/strong positive (scored as 1+ to 2+). Of the 339 NSCLCs, SYNGR4 was strongly stained in 127 (37.5%) cases (score 2+), weakly stained in 157 (46.3%) cases (score 1+), and not stained in 55 (16.2%) cases (score 0) (Table 1A). Then, it was examined a correlation of SYNGR4 expression (strong positive vs weak positive/absent) with various clinicopathologic parameters and found its significant correlation with gender (higher in male; P=0.0487 by Fisher's exact test), histological type (higher in non-ADC; P=0.0116 by Fisher's exact test), and lymph-node metastasis (higher in pN1-2; P=0.0175 by Fisher's exact test) (Table 1A). The median survival time of NSCLC patients was significantly shorter in accordance with the higher expression levels of SYNGR4 (P=0.0002, log-rank test; FIG. 2C, right panel). The invention also applied univariate analysis to evaluate associations between patient prognosis and several factors, including age, sex, pathologic tumor stage (tumor size; T1 vs T2-3), pathologic node stage (node status; NO vs N1-2), histology (ADC vs other histology types), and SYNGR4 status (score 0, 1+ vs score 2+). All those variables were significantly associated with poor prognosis. Multivariate analysis using a Cox proportional hazard model determined that SYNGR4 (P=0.0078) as well as other three factors (age, tumor size, and lymph node metastasis) were independent prognostic factors for surgically treated NSCLC patients (Table 1B).

TABLE 1A
Association between SYNGR4-positivity in NSCLC tissues
and patients' characteristics (n = 339)

| | | SYNGR4 expression | | | |
|---|---|---|---|---|---|
| | | Strong | Low or absent | | P value |
| | Total | expression | expression | | Strong vs |
| | n = 339 | n = 128 | n = 211 | Chi-square | Low or absent |
| Sex | | | | | |
| Female | 100 | 29 | 71 | 3.841 | 0.0487* |
| Male | 239 | 98 | 141 | | |
| Age (year) | | | | | |
| >=65 | 177 | 67 | 110 | 0.002 | NS |
| <65 | 162 | 60 | 102 | | |
| Smoking status | | | | | |
| never smoker | 92 | 30 | 62 | 1.002 | NS |
| current or ex-smoker | 247 | 97 | 150 | | |
| T factor | | | | | |
| T1 | 139 | 44 | 95 | 2.987 | NS |
| T2 + T3 | 200 | 83 | 117 | | |
| N factor | | | | | |
| N0 | 225 | 74 | 151 | 5.411 | 0.0175* |
| N1 + N2 | 114 | 53 | 61 | | |
| Histological type | | | | | |
| ADC | 204 | 65 | 139 | 6.272 | 0.0116* |
| non-ADC | 135 | 62 | 73 | | |

*P < 0.05 (Fisher's exact test)
NS, no significance
ADC, adenocarcinoma
non-ADC, squamouse cell carcinoma plus large cell carcinoma and adenosquamous cell carcinoma

TABLE 1B
Cox's proportional hazards model analysis of
prognostic factors in patients with NSCLCs

| Variables | Hazards ratio | 95% CI | Unfavorable/Favorable | P-value |
|---|---|---|---|---|
| Univariate analysis | | | | |
| SYNGR4 | 1.883 | 1.339-2.650 | Positive/Negative | 0.0003* |
| Age (years) | 1.693 | 1.189-2.410 | >=65/65> | 0.0035* |
| Gender | 1.653 | 1.100-2.485 | Male/Female | 0.0155* |
| Smoking status | 1.249 | 0.838-1.859 | Current or ex-smoker/never smoker | NS |
| pT factor | 2.395 | 1.621-3.540 | T2 + T3/T1 | <0.0001* |
| pN factor | 2.225 | 1.580-3.132 | N1 + N2/N0 | <0.0001* |
| Histological type | 1.515 | 1.076-2.131 | non-ADC/ADC | 0.0172* |
| Multivariate analysis | | | | |
| SYNGR4 | 1.602 | 1.132-2.267 | Positive/Negative | 0.0078* |
| Age (years) | 1.811 | 1.252-2.590 | >=65/65> | 0.0015* |
| Gender | 1.361 | 0.867-2.138 | Male/Female | NS |
| pT factor | 1.811 | 1.192-2.751 | T2 + T3/T1 | 0.0054* |
| pN factor | 2.077 | 1.454-2.967 | N1 + N2/N0 | <0.0001* |
| Histological type | 0.989 | 0.673-1.453 | non-ADC/ADC | NS |

ADC, adenocarcinoma
non-ADC, squamous-cell carcinoma plus large-cell carcinoma and adenosquamous-cell carcinoma
NS, no significance
*P < 0.05

### Example 4

### Cell Growth Effect of SYNGR4

To assess whether up-regulation of SYNGR4 plays a role in growth and/or survival of lung cancer cells, it was evaluated the inhibition of endogenous SYNGR4 expression by siRNA against SYNGR4, along with two different control siRNAs (siRNAs for EGFP and LUC). Treatment of NSCLC cells (A549) (Left panels) and SCLC cells (SBC-5) (Right panels) with the effective siRNA could reduce expression of SYNGR4 (FIG. 3A), and resulted in significant inhibition of cell viability and colony numbers measured by MTT and colony formation assays (FIG. 3A). To disclose the role of SYNGR4 in tumorigenesis, plasmids expressing SYNGR4 or mock plasmids transfected into COS-7 cells and evaluated the effect of SYNGR4 on cell growth, and observed significant cell proliferation in COS-7 cells exogenously overexpressing SYNGR4 (FIG. 3B). In accordance with the result of siRNA assays, the data are consistent with the conclusion that SYNGR4 is required for the tumor growth and/or survival.

### Example 5

### Promotion of Mammalian Cell Invasion by SYNGR4

Since strong SYNGR4 expression was associated with lymph node metastasis and poorer prognosis for lung cancer patients, the role of SYNGR4 in cellular invasion in mammalian cells was examined by Matrigel assays. Transfection of SYNGR4 expressing vector into COS-7 or NIH3T3 cells significantly enhanced its invasion through Matrigel, compared with cell transfected with mock vector (FIG. 4A).

### Example 6

### Inhibitory Effect of anti-SYNGR4 Antibody on the Cell Invasive Activity

Since it was found that SYNGR4 was expressed on cell surface, the function of SYNGR4 was blocked by using antibody for SYNGR4. C-terminus of SYNGR4 seemed to be outside of cell membrane, so this lesion was targeted by antibody treatment. This present inventors applied COS-7 cells transfected with SYNGR4 expressing vector or mock vector to Matrigel assays for the evaluation of inhibition of SYNGR4-dependent cellular invasion by SYNGR4 antibody. The invasive activity induced by SYNGR4 was significantly blocked by anti-SYNGR4 antibody in a dose dependent manner (FIG. 4B). It was then evaluated the functional blocking effect of anti-SYNGR4 antibody on lung cancer cell invasion. In concordance with the result of COS-7 cells, cellular invasiveness of A549 cells, which highly expressed endogenous SYNGR4, were effectively blocked by anti-SYNGR4 antibody in a dose dependent manner, whereas the antibody failed to block cellular invasiveness of SYNGR4 negative NCI-H1781 cells (FIG. 4C). These results are consistent with the conclusion that SYNGR4 is required for cell invasion and is an ideal target for antibody-based immunotherapy.

Interaction of SYNGR4 with GRB2 through GRB2 SH2 domain binding motif on SYNGR4.

As demonstrated, SYNGR4 is a membrane protein and both N- and C-terminal is outside of cell surface. The present inventors next evaluated the posttranscriptional modification in cell-inside region of SYNGR4. Because SYNGR4 family protein is heavily phosphorylated (Janz R et al. Neuron 1999;24:687-700., Janz R, J Biol Chem. 1998; 273: 2851-7.), the inventors first treated phosphatase COS-7 cells exogenously expressing SYNGR4 and found that SYNGR4 was likely to be phosphorylated, because the band was lower shifted after the treatment (FIG. 5A, left upper panel). The inventors next tried to identify phosphorylated residue of SYNGR4 protein by immunoblotting of immunoprecipitated exogenous SYNGR4-expressed COS-7 cell lysate using anti-phosphorylated tyrosine (FIG. 5A, right panel), and confirmed that tyrosine residue in SYNGR4 could be phosphorylated. The inventors next focused on tyrosine residue in intracellular sequence of SYNGR4 protein, and found that tyrosine-46 intracellularly located (FIG. 5A, lower panel). Since SYNGR4 tyrosine-46 was included in a predicted consensus GRB2 SH2 domain binding motif (pY—X—N), the inventors next evaluated the possibility that SYNGR4 interacts with GRB2, a multifunctional adaptor protein that interacts with various proteins. Their interaction was confirmed by immunoprecipitation experiment (FIG. 5B), which indicates that SYNGR4 might be involved in functional signaling pathway using GRB2. To examine whether tyrosine-46 in SYNGR4 could be phosphorylated and function as GRB2-interacting residue, the inventors next generated phenylalanine-replaced mutant SYNGR4 and performed immunoblotting using anti-phosphorylated tyrosine using wild type or mutant SYNGR4 immunoprecipitants obtained by anti-Flag antibody. Expectedly, blotted phospho-tyrosine was markedly decreased (FIG. 5C, left panel), indicating that tyrosine-46 could be phosphorylated. Next immunoblotting of GRB2 was performed using the same immunoprecipitants and found that the amount of GRB2-binding SYNGR4 was decreased in mutant SYNGR4-Y46F compared with wild type SYNGR4 (FIG. 5C, left panel). These data indicates that tyrosine-46 in SYNGR4 is important residue for the interaction of SYNGR4 with GRB2.

SYNGR4 as a novel modulator of MAPK signaling pathway through PAK1.

Since introduction of SYNGR4 into mammalian cells exhibited promotion of cell growth and invasion, the inventors attempted to find SYNGR4-dependent signaling molecules related to cell growth and invasion. GRB2 is known to be a key molecule that mediates signals of cell surface to Ras-MAPK pathway by cooperating with SOS (Downward J. FEBS Lett. 1994; 338: 113-7). Because Ras-MAPK signaling is considered as one of the most causative signals of lung cancer progression (Sebolt-Leopold J S, Nat Rev Cancer. 2004; 4; 937-47), the inventors first evaluated the effect of exogenous SYNGR4 expression was on the activation of RAS-MAPK signaling molecules in COS-7 cells. The present inventors found no increase of the levels of activated RAS by pull-down assay using RAF1 recombinant protein (FIG. 7B), but interestingly phosphorylation of c-Raf, MEK, and ERK proteins were significantly elevated by exogenously expressed SYNGR4 (FIG. 6A, left panel). Furthermore by knocking down endogenous SYNGR4 expression by siRNA against SYNGR4 in lung cancer cell lines, A549 and SBC-3 cells, the inventors found marked decrease in phosphorylation of each MAPK signaling proteins (FIG. 6A, right panels). These data suggested that MAPK signaling is a target of SYNGR4 but not through RAS activation. The inventors next performed assay knocking down endogenous GRB2 protein in COS-7 cells by siRNA against GRB2, followed by introduction of SYNGR4 (FIG. 6B). It was found that phosphorylation status of MAPK signaling proteins was not altered by introduction of SYNGR4 in siGRB2-treated cells, although significant reduction of baseline phosphorylation status by si-GRB2 was found, probably because of termination of GRB2-SOS-RAS signaling pathway. The inventors further analyzed the relationship of SYNGR4 with GRB2 by introducing mutant SYNGR-Y46F whose binding affinity to GRB2 was significantly reduced into COS-7 cells. Expectedly, phosphorylation status of MAPK signaling molecules was significantly decreased in cells transfected with mutant SYNGR-Y46F expression vector compared with those transfected with plasmids expressing wild type SYNGR4 (FIG. 6C). These data indicates that GRB2 is indispensable interacting protein for SYNGR4 to function as it's a downstream signaling molecule. Next the present inventors searched for other molecules except RAS that affects MAPK signaling molecule. Among phosphorylation site in MAPK signaling molecules, serine-298 of MEK1 is known as a site which is specifically phosphorylated by p21 protein-activated kinase (PAK) (Slack-Davis J K, et al. J Cell Biol. 2003; 162: 281-91., Park E R et al. Cell Signal. 2007; 19: 1488-96.), and the inventors found the levels of serine-298 of MEK1 phosphorylation to be enhanced in concordance with the expression of SYNGR4 (FIGS. 6A and 6C). Therefore, the phosphorylation status of PAK1-Thr423 was evaluated, which is known as an indispensable phosphorylation site for its kinase activity in lung cancer cells by siRNA for SYNGR4 and found that PAK1 activity was decreased (FIG. 6D). Next it was found that knocking down of endogenous PAK1 by siRNA for PAK1 reduced enhancement of phosphorylation induced by exogenous SYNGR4 in COS-7 cells (FIG. 6E). The results suggested that SYNGR4 could exert oncogenic function possibly with GRB2-PAK1 and subsequent MAPK signal activation. Finally the inventors evaluated whether the enhancement of growth and invasive activity induced by SYNGR4 is inhibited by replacement of tyrosine-46 to phenylalanine in SYNGR4. COS-7 cells exogenously expressing mutant SYNGR-S46F exhibited loss of ability to enhance growth and invasive activity compared with wild type SYNGR4 introduced cells (FIG. 6F). According to these findings it could be suggested alternative growth and invasion-promoting pathway involving SYNGR4, GRB2, PAK1 (FIG. 6G).

Discussion

Recent accumulation of knowledge in cancer genomics and molecular biochemistry introduced new strategy for treatment of cancer like molecular target drugs (Daigo Y et al., Gen Thorac Cardiovasc Surg 2008, 56: 43-53). Molecular targeted drugs are expected to be highly specific to malignant cells, with minimal adverse effects due to their well-defined mechanisms of action. To find such molecules, it was established a powerful screening system to identify proteins that were activated specifically in lung cancer cells. The strategy was as follows: (a) identification of up-regulated genes in 101 lung cancer samples through the genome-wide cDNA microarray system, containing more than 32,256 genes, coupled with laser microdissection (Daigo Y et al., Gen Thorac Cardiovasc Surg 2008, 56: 43-53; Kikuchi T et al., Oncogene 2003, 22: 2192-205; Kakiuchi S et al., Mol Cancer Res 2003, 1: 485-99; Kakiuchi S et al., Hum Mol Genet 2004, 13: 3029-43; Kikuchi T et al., Int J Oncol 2006, 28: 799-805; Taniwaki M et al., Int J Oncol 2006, 29: 567-75); (b) verification of very low or absent expression of such genes in normal organs by cDNA microarray analysis and multiple-tissue Northern blot analysis; (c) confirmation of the clinicopathologic significance of their overexpression using tissue microarray consisting of hundreds of NSCLC tissue samples (Suzuki C et al., Cancer Res 2003, 63: 7038-41; Ishikawa Net al., Clin Cancer Res 2004, 10: 8363-70; Kato T et al., Cancer Res 2005, 65: 5638-46; Furukawa C et al., Cancer Res 2005, 65: 7102-10; Ishikawa N et al., Cancer Res 2005, 65: 9176-84; Suzuki C et al., Cancer Res 2005, 65: 11314-25; Ishikawa N et al., Cancer Sci 2006, 97: 737-45; Takahashi K et al., Cancer Res 2006, 66: 9 408-19; Hayama S et al., Cancer res 2006, 66: 10339-48; Kato T et al., Clin Cancer Res 2007, 13: 434-42; Suzuki C et al., Mol Cancer Ther 2007, 6: 542-51; Yamabuki T et al., Cancer Res 2007, 67: 2517-25; Hayama S et al., Cancer Res 2007, 67: 2517-25; Kato T et al., Cancer Res 2007, 67: 8544-53; Taniwaki M et al., Clin Cancer Res 2007, 13: 6624-31; Ishikawa N et al., 2007, 67: 11601-11; Mano T et al., Cancer Sci 2007, 98: 1902-13); and (d) verification of the targeted genes whether they are essential for the survival or growth of lung cancer cells by siRNA (Suzuki C et al., 2003, 63: 7038-41; Ishikawa N et al., Clin Cancer Res 2004, 10: 8363-70; Kato T et al., Cancer Res 2005, 65: 5638-46; Furukawa C et al., Cancer Res 2005, 65: 7102-10; Suzuki C et al., Cancer Res 2005, 65: 11314-25; Ishikawa N et al., Cancer Sci 2006, 97: 737-45; Takahashi K et al., Cancer Res 2006, 66: 9408-19; Hayama S et al., Cancer Res 2006, 66: 10339-48; Kato T et al., Clin Cancer Res 2007, 13: 434-42; Suzuki C et al., Mol Cancer Ther 2007, 6: 542-51; Yamabuki T et al., Cancer Res 2007, 67: 2517-25; Hayama S et al., Cancer Res 2007, 67: 4113-22; Kato T et al., Cancer Res 2007, 67: 8544-53; Taniwaki M et al., Clin Cancer Res 2007, 13: 6624-31; Ishikawa N et al., Cancer Res 2007, 67: 11601-11; Mano Yet al., Cancer Sci 2007, 98: 1902-13; kato T et a;., Clin Cancer Res 2008, 14:2263-70). Through the analyses, it was identified several genes encoding oncoantigens that are candidates for the development of diagnostic markers, therapeutic drugs, and/or immunotherapy. Among them, genes encoding tumor-specific transmembrane or secretory proteins are considered to have significant advantages because they are present on the cell surface or within the extracellular space. The present invention is based, in part, on the discovery that one of the genes, SYNGR4, that encodes a multi-pass transmembrane protein, and shown that SYNGR4 is frequently overexpressed in clinical lung cancer samples and cell lines, and that its gene product plays important roles in the growth and invasion of lung cancer cells. SYNGR4 is a 25 kD protein that first described its chromosomal localization by transcript mapping of 19 q-arm glioma tumor suppressor region (Smith J S et al., Genomics 2000, 64: 44-50; Kedra D et al., Hum Genet 1998, 273: 2851-7), but there has been no report that refers its biological function as well as its involvement in carcinogenesis.

In this example, it was found that strong SYNGR4 expression was associated with poorer clinical outcome for NSCLC patients. It was also demonstrated that inhibition of endogenous expression of SYNGR4 by siRNA resulted in marked reduction of viability of lung cancer cells. Exogenous expression of SYNGR4 enhanced the cell growth and cellular migration/invasive activity in mammalian cells. Furthermore, it was revealed that Tyr46 in SYNGR4 was phosphorylated and important for binding with GRB2. GRB2 is a key molecule for transmitting the stimulation of cell surface to cytoplasm signaling pathways (Downward J. FEBS Lett. 1994; 338: 113-7.), and there are several interacting proteins of GRB2 related to downstream signaling pathways leading to cell growth and invasion. Because MAPK signaling is considered to have critical role for cancer cell proliferation (Sebolt-Leopold J S and Herrera R. Nat Rev Cancer. 2004; 4; 937-47.), the inventors focused on this signaling pathway to elucidate the function of SYNGR4. Expectedly, activity of MAPK signaling molecules was enhanced by expression of SYNGR4, and was suppressed by siRNA for SYNGR4.

Phosphorylation levels of serine-298 in MEK1, which is known as a specific phosphorylation site by PAK1 kinase (Slack-Davis J K, et al. J Cell Biol. 2003; 162: 281-91., 53), was increased or decreased in concordance with SYNGR4 expression. In addition, serine 338 in c-RAF, which is known to be phosphorylated by PAK1 and enhance c-RAF activity in cooperating with RAS (Chaudhary A, et al. Curr Biol 2000; 10: 551-4.), was also altered its phosphorylation status in concordance with SYNGR4 expression. Another evidence indicates that GRB2 could directly interact with and activate PAK1 (Puto L A, et al. J Biol Chem. 2003; 278: 9388-93.). The inventors thus hypothesized that SYNGR4 may positively regulate MAPK signaling pathway via PAK1. Consistently, knocking down of PAK1 by siRNA for PAK1 revealed reduced the effect of SYNGR4 on the enhancement of phosphorylation of MAPK signal proteins without total elimination of phosphorylation in each protein, which is consistent with the fact that PAK1-mediated regulation of c-RAF and MEK1 is likely to be important for maximization of canonical growth factor and RAS-mediated regulation of MAPK signaling pathway (Beeser A, et al. J Biol Chem. 2005; 280: 36609-15.). Furthermore, PAK1 is one of the effectors of Rac/Cdc42 GTPases and its activity is closely related to cell invasion, cytoskeletal dynamics and cell motility (Kumar R, et al. Nat Rev Cancer. 2006; 6: 459-71.).

d type and Y46F SYNGR4-introduced COS-7 cells and decreased ability was found in siRNA for SYNGR4-treated lung cancer cells. These data supports our hypothesis that SYNGR4 promotes cell invasion via GRB2-PAK1 pathway.

Although the detailed function of SYNGR4 in pulmonary carcinogenesis is still under analyses, our results are consistent with the conclusion that SYNGR4 expression promotes progression of lung tumors by stimulating cell proliferation/survival and metastasis.

Since SYNGR4 is expressed in only testis among normal tissues and a membrane protein is considered to be ideal target for antibody-based therapy, the efficacy of anti-SYNGR4 antibody in blocking SYNGR4-dependent invasive activity in SYNGR4-positive cells was examined, and it was observed that cell invasion was significantly suppressed by an anti-SYNGR4 antibody. This finding supports the use of anti-SYNGR4 antibody for lung cancer therapy.

This invention demonstrates that SYNGR4 cancer-testis antigen is frequently expressed in lung cancers and is al prognostic biomarker for this disease. SYNGR4 overexpression in resected specimens may be a useful index for application of adjuvant therapy to the lung cancer patients who are likely to show poor prognosis. Moreover, SYNGR4 is likely to be an essential contributor to aggressive features of NSCLC and a likely target for the development of new therapeutic approaches, such as molecular targeted drugs and antibody-based immunotherapy to lung cancer.

### INDUSTRIAL APPLICABILITY

As demonstrated herein, cell growth is suppressed by a double-stranded molecule that specifically targets the SYNGR4 gene. Thus, the novel double-stranded molecules are useful candidate for the development of an anti-cancer pharmaceutical. For example, agents that block the expression of SYNGR4 protein and/or prevent its activity may find therapeutic utility as an anti-cancer agent, particularly an anti-cancer agent for the treatment of lung cancer, more particularly for the treatment of NSCLC and SCLC.

The expression of human gene SYNGR4 is markedly elevated in lung cancer, as compared to normal organs. Accordingly, the gene can be conveniently used as diagnostic marker of lung cancer and the protein encoded thereby find utility in diagnostic assays of lung cancer.

Furthermore, the methods described herein are also useful in diagnosis of lung cancer, including small-cell lung carcinomas (SCLCs) and non-small cell lung cancers (NSCLCs), as well as the prediction of the poor prognosis of the patients with these diseases. Moreover, the present invention provides a likely candidate for development of therapeutic approaches for cancer including lung cancers.

Furthermore, SYNGR4 polypeptide is a useful target for the development of anti-cancer pharmaceuticals. For example, agents that bind SYNGR4 or block the expression of SYNGR4 or prevent its activity may find therapeutic utility as anti-cancer agents, particularly anti-cancer agents for the treatment of lung cancer.

All publications, databases, sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. A method for diagnosing lung cancer, said method comprising the steps of:
(a) determining the expression level of the gene in a subject-derived biological sample by any one of the method methods selected from the group consisting of:
(i) detecting the mRNA of SYNGR4,
(ii) detecting the SYNGR4 protein;
(iii) detecting the biological activity of the SYNGR4 protein; and
(b) correlating an increase in the expression level determined in step (a) as compared to a normal control level of the gene to the presence of lung cancer.

2. The method of claim 1, wherein the expression level determined in step (a) is at least 10% greater than the normal control level.

3. The method of claim 1, wherein the expression level determined in step (a) is determined by detecting the binding of an antibody against the SYNGR4 protein.

4. The method of claim 1, wherein the subject-derived biological sample comprises biopsy, sputum, blood, pleural effusion or urine.

5. A method for assessing or determining the prognosis of a patient with lung cancer, which method comprises the steps of:
(a) detecting the expression level of a gene in a patient-derived biological sample;
(b) comparing the detected expression level to a control level; and
(c) determining the prognosis of the patient based on the comparison of (b) and wherein the gene is SYNGR4.

6. The method of claim 5, wherein the control level is a good prognosis control level and an increase of the expression level compared to the control level is determined as poor prognosis.

7. The method of claim 5, wherein the increase is at least 10% greater than the control level.

8. The method of claim 5, wherein the expression level is determined by any one method selected from the group consisting of:
(a) detecting mRNA of SYNGR4;
(b) detecting the SYNGR4 protein; and
(c) detecting the biological activity of the SYNGR4 protein.

9. The method of claim 5, wherein the patient derived biological sample comprises biopsy, sputum or blood, pleural effusion or urine.

10. A kit for diagnosing lung cancer or assessing or determining the prognosis of a patient with lung cancer, which comprises a reagent selected from the group consisting of:
(a) a reagent for detecting mRNA of a gene;
(b) a reagent for detecting the protein encoded by the gene; and
(c) a reagent for detecting the biological activity of the protein, wherein the gene is SYNGR4.

11. The kit of claim 10, wherein the reagent is a probe to a gene transcript of the gene.

12. The kit of claim 10, wherein the reagent is an antibody against the protein encoded by the gene.

13. An isolated double-stranded molecule that, when introduced into a cell, inhibits in vivo expression of SYNGR4 as well as cell proliferation, wherein said molecule comprises a sense strand and an antisense strand complementary thereto, and wherein said strands hybridize to each other to form the double-stranded molecule.

14. The double-stranded molecule of claim 13, wherein the sense strand comprises the sequence corresponding to a target sequence selected from the group consisting of SEQ ID NOs: 11, 12, 19 and 20.

15. The double-stranded molecule of claim 14, wherein the sense strand hybridizes with the antisense strand at the target sequence to form the double-stranded molecule having between 19 and 25 nucleotide pair in length

16. The double-stranded molecule of claim 13, which consists of a single polynucleotide comprising both the sense and antisense strands linked by an intervening single strand.

17. The double-stranded molecule of claim 16, which has the general formula 5′-[A]-[B]-[A′]-3′, wherein [A] is the sense strand comprising a sequence corresponding to a target sequence selected from the group consisting of SEQ ID NOs: 11, 12, 19 and 20, [B] is the intervening single-strand consisting of 3 to 23 nucleotides, and [A′] is the antisense strand comprising a sequence complementary to [A].

18. A vector encoding the double-stranded molecule of claim 13.

19. Vectors comprising each of a combination of polynucleotide comprising a sense strand nucleic acid and an antisense strand nucleic acid, wherein said sense strand nucleic acid comprises the nucleotide sequence of SEQ ID NO: 11, 12, 19 or 20, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the SYNGR4 gene, inhibit expression of said gene.

20. A method for treating a cancer expressing at least one gene selected from the group consisting of SYNGR4 gene, wherein the method comprises the step of administering the double-stranded molecule of claim 13, a vector encoding the double-stranded molecule of claim 13, or vectors comprising each of a combination of polynucleotide comprising a sense strand nucleic acid and an antisense strand nucleic acid, wherein said sense strand nucleic acid comprises the nucleotide sequence of SEQ ID NO: 11, 12, 19 or 20, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the SYNGR4 gene, inhibit expression of said gene.

21. The method of claim 20, wherein the cancer to be treated is lung cancer.

22. A composition for treating a cancer expressing SYNGR4 gene, wherein said composition comprises the isolated double-stranded molecule of claim 13, a vector encoding the double-stranded molecule of claim 13, or vectors comprising each of a combination of polynucleotide comprising a sense strand nucleic acid and an antisense strand nucleic acid, wherein said sense strand nucleic acid comprises the nucleotide sequence of SEQ ID NO: 11, 12, 19 or 20, and said antisense strand nucleic acid consists of a sequence complementary to the sense strand, wherein the transcripts of said sense strand and said antisense strand hybridize to each other to form a double-stranded molecule, and wherein said vectors, when introduced into a cell expressing the SYNGR4 gene, inhibit expression of said gene.

23. The composition of claim 22, wherein the cancer to be treated is lung cancer.

24. A method of screening for a candidate compound for treating or preventing lung cancer, or inhibiting lung cancer cell growth, said method comprising the steps of:
(a) contacting a test compound with a polypeptide encoded by a polynucleotide of SYNGR4;
(b) detecting the binding activity between the polypeptide and the test compound; and
(c) selecting a compound that binds to the polypeptide.

25. A method of screening for a candidate compound for treating or preventing lung cancer, or inhibiting lung cancer cell growth, said method comprising the steps of
(a) contacting a test compound with a polypeptide encoded by a polynucleotide of SYNGR4;
(b) detecting the biological activity of the polypeptide of step (a); and
(c) selecting the test compound that suppresses the biological activity of the polypeptide encoded by the polynucleotide of SYNGR4 as compared to the biological activity of said polypeptide detected in the absence of the test compound.

26. The method of claim 25, wherein the biological activity is selected from the group consisting of the facilitation of the cell proliferation and cell invasion.

27. A method of screening for a candidate compound for treating or preventing lung cancer or inhibiting lung cancer cell growth, said method comprising the steps of:
(a) contacting a candidate compound with a cell expressing SYNGR4; and
(b) selecting the candidate compound that reduces the expression level of SYNGR4 in comparison with the expression level detected in the absence of the test compound.

28. A method of screening for a candidate compound for treating or preventing lung cancer or inhibiting lung cancer cell growth, said method comprising the steps of:
(a) contacting a candidate compound with a cell into which a vector, comprising the transcriptional regulatory region of SYNGR4 and a reporter gene that is expressed under the control of the transcriptional regulatory region, has been introduced;
(b) measuring the expression or activity of said reporter gene; and
(c) selecting a candidate compound that reduces the expression or activity level of said reporter gene as compared to a control.

29. A composition for treating or preventing lung cancer, said composition comprising a pharmaceutically effective amount of an anti SYNGR4 antibody or fragment thereof.

30. A method for treating or preventing lung cancer in a subject, comprising administering to said subject an anti SYNGR4 antibody or fragment thereof.

31. A method of screening for a candidate compound for inhibiting a binding between a SYNGR4 polypeptide and a GRB2 polypeptide, or treating or preventing a cancer, said method comprising the steps of:
(a) contacting an SYNGR4 polypeptide or functional equivalent thereof with a GRB2 polypeptide or functional equivalent thereof in presence of a test compound;
(b) detecting a binding between the polypeptides;
(c) comparing binding level detected in the step (b) with those detected in absence of the test compound; and
(d) selecting the test compound that reduces or inhibits binding level comparing with those detected in absence of the test compound in step (c).

32. The method of claim 31, wherein the functional equivalent of SYNGR4 comprises Tyrosine-46.

33. A method of screening for a candidate compound for inhibiting the phosphorylation of SYNGR4, or treating or preventing a cancer, comprising the steps of:
(a) contacting an SYNGR4 polypeptide or functional equivalent thereof with a test compound under a condition that allows phosphorylation of the polypeptide;
(b) detecting the phosphorylation level at tyrosine-46 residue of the polypeptide described in (a);
(c) comparing the phosphorylation level of tyrosine-46 residue in the polypeptide with the phosphorylation level of tyrosine-46 residue in the protein detected in the absence of the compound; and
(d) selecting the compound that reduced reduces the phosphorylation level of tyrosine-46 residue of the polypeptide as the candidate compound.

34. A method of screening for a candidate compound for inhibiting the activity of SYNGR4 for phosphorylating down-stream effectors, or treating or preventing cancer comprising the steps of:
(a) contacting a test compound with a polypeptide encoded by a polynucleotide of SYNGR4 in the presence of a polypeptide encoded by a polynucleotide of GRB2 and/or PAK1, under the condition for phosphorylation of at least one of down-stream effectors of SYNGR4 selected from the group consisting of PAK1, c-Raf, MEK1, MEK1/2 and ERK1/2;
(b) detecting the phosphorylation level of the down-stream effector of SYNGR4; and
(c) selecting the test compound that suppresses the phosphorylation level of the down-stream effector of SYNGR4 as compared to the phosphorylation level of the down-stream effector of SYNGR4 detected in the absence of the test compound.

35. The method of claim 34, wherein the phosphorylation level of the down-stream effector of SYNGR4 to be detected is that of Thr423 of PAK1, Ser338 of c-Raf, Ser 298 of MEK1, Ser217/221 of MEK1/2, and Thr202/204 of ERK1/2, respectively.

36. The method of claim 31, wherein the cancer is lung cancer.

37. The method of claim 33, wherein the cancer is lung cancer.

38. The method of claim 34, wherein the cancer is lung cancer.
